SEQUENCE LISTING (80 sequences)

SEQ ID NO: 1 - protein, 14 aa, synthetic construct
WWCNGNSENWTCTW

SEQ ID NO: 2 - protein, 14 aa, synthetic construct
WGCTGTWGNETCWW

SEQ ID NO: 3 - protein, 14 aa, synthetic construct
WDCWGNTDDWTCTW

SEQ ID NO: 4 - protein, 14 aa, synthetic construct
YGCYGDRNDYTCTW

SEQ ID NO: 5 - protein, 14 aa, synthetic construct
LGCHGDNSEWTCVW

SEQ ID NO: 6 - protein, 19 aa, synthetic construct
WSCKWWGGAADEYTYSCGW

SEQ ID NO: 7 - protein, 14 aa, synthetic construct
YGCTGDAYEYTCTW

SEQ ID NO: 8 - protein, 14 aa, synthetic construct
WTCYWTEGVSGCRW

SEQ ID NO: 9 - protein, 15 aa, synthetic construct
YWRCDWYEETVACGT

SEQ ID NO: 10 - protein, 14 aa, synthetic construct
WSCHYSYWTVSCGT

SEQ ID NO: 11 - protein, 14 aa, synthetic construct
YGCSMDWRGVTCAW

SEQ ID NO: 12 - protein, 14 aa, synthetic construct
DYCLWAPDAWGCQN

SEQ ID NO: 13 - protein, 14 aa, synthetic construct
RGCTGNVYDWTCVW

SEQ ID NO: 14 - protein, 14 aa, synthetic construct
WTCHGNADEWTCSW

SEQ ID NO: 15 - protein, 14 aa, synthetic construct
DGCKGDWYEWTCWW

SEQ ID NO: 16 - protein, 14 aa, synthetic construct
YGCRGGDGEWTCVW

SEQ ID NO: 17 - protein, 14 aa, synthetic construct
DYCYGDVEEWTCVY

SEQ ID NO: 18 - protein, 14 aa, synthetic construct
YGCETYSGGWTCLW

SEQ ID NO: 19 - protein, 14 aa, synthetic construct
YGCKWDWKGWTCAW

SEQ ID NO: 20 - protein, 14 aa, synthetic construct
YGCSVNADGWTCSW

SEQ ID NO: 21 - protein, 14 aa, synthetic construct
YGCYGEQEQWTCLW

SEQ ID NO: 22 - protein, 14 aa, synthetic construct
YGCQYYGWDYTCAW

SEQ ID NO: 23 - protein, 14 aa, synthetic construct
YGCWGNYNDYTCSW

SEQ ID NO: 24 - protein, 14 aa, synthetic construct
ETCWYLYGREWCVG

SEQ ID NO: 25 - protein, 14 aa, synthetic construct
MRCYPWWYGPMCVR

SEQ ID NO: 26 - protein, 14 aa, synthetic construct
WGCLGMGYDRTCWW

SEQ ID NO: 27 - protein, 14 aa, synthetic construct
WTCWTVYGKSSCWD

SEQ ID NO: 28 - protein, 14 aa, synthetic construct
YGCWWDAYGSTCTW

SEQ ID NO: 29 - protein, 14 aa, synthetic construct
YGCDLWKGWTVCGY

SEQ ID NO: 30 - protein, 14 aa, synthetic construct
RYCTHTQTYVQCQD

SEQ ID NO: 31 - protein, 14 aa, synthetic construct
WGCNLDWYGWTCWW

SEQ ID NO: 32 - protein, 14 aa, synthetic construct
WSCVVYGDNWTCSS

SEQ ID NO: 33 - protein, 14 aa, synthetic construct
WSCQLGSGWWTCAW

SEQ ID NO: 34 - protein, 14 aa, synthetic construct
WTCWETTEGYGCNW

SEQ ID NO: 35 - protein, 14 aa, synthetic construct
WGCTGGAWEYTCWW

SEQ ID NO: 36 - protein, 14 aa, synthetic construct
YGCSVTWGGYTCTW

SEQ ID NO: 37 - protein, 14 aa, synthetic construct
YNCWYKMGWYVCWS

SEQ ID NO: 38 - protein, 11 aa, synthetic construct
CRAYSNWFYIC

SEQ ID NO: 39 - protein, 20 aa, synthetic construct
AETVEFWWCNGNSENWTCTW

SEQ ID NO: 40 - protein, 20 aa, synthetic construct
AETVEFWGCTGTWGNETCWW

SEQ ID NO: 41 - protein, 20 aa, synthetic construct
AETVEFWDCWGNTDDWTCTW

SEQ ID NO: 42 - protein, 20 aa, synthetic construct
AETVEFYGCYGDRNDYTCTW

SEQ ID NO: 43 - protein, 20 aa, synthetic construct
AETVEFLGCHGDNSEWTCVW

SEQ ID NO: 44 - protein, 25 aa, synthetic construct
AETVEFWSCKWWGGAADEYTYSCGW

SEQ ID NO: 45 - protein, 20 aa, synthetic construct
AETVEFYGCTGDAYEYTCTW

SEQ ID NO: 46 - protein, 20 aa, synthetic construct
AETVEFWTCYWTEGVSGCRW

SEQ ID NO: 47 - protein, 20 aa, synthetic construct
AETVEYWRCDWYEETVACGT

SEQ ID NO: 48 - protein, 20 aa, synthetic construct
AETVEFWSCHYSYWTVSCGT

SEQ ID NO: 49 - protein, 20 aa, synthetic construct
AETVEFYGCSMDWRGVTCAW

SEQ ID NO: 50 - protein, 20 aa, synthetic construct
AETVEFDYCLWAPDAWGCQN

SEQ ID NO: 51 - protein, 20 aa, synthetic construct
AETVEFRGCTGNVYDWTCVW

SEQ ID NO: 52 - protein, 20 aa, synthetic construct
AETVEFWTCHGNADEWTCSW

SEQ ID NO: 53 - protein, 20 aa, synthetic construct
AETVEFDGCKGDWYEWTCWW

SEQ ID NO: 54 - protein, 20 aa, synthetic construct
AETVEFYGCRGGDGEWTCVW

SEQ ID NO: 55 - protein, 20 aa, synthetic construct
AETVEFDYCYGDVEEWTCVY

SEQ ID NO: 56 - protein, 20 aa, synthetic construct
AETVEFYGCETYSGGWTCLW

SEQ ID NO: 57 - protein, 20 aa, synthetic construct
AETVEFYGCKWDWKGWTCAW

SEQ ID NO: 58 - protein, 20 aa, synthetic construct
AETVEFYGCSVNADGWTCSW

SEQ ID NO: 59 - protein, 20 aa, synthetic construct
AETVEFYGCYGEQEQWTCLW

SEQ ID NO: 60 - protein, 20 aa, synthetic construct
AETVEFYGCQYYGWDYTCAW

SEQ ID NO: 61 - protein, 20 aa, synthetic construct
AETVEFYGCWGNYNDYTCSW

SEQ ID NO: 62 - protein, 20 aa, synthetic construct
AETVEFETCWYLYGREWCVG

SEQ ID NO: 63 - protein, 20 aa, synthetic construct
AETVEFMRCYPWWYGPMCVR

SEQ ID NO: 64 - protein, 20 aa, synthetic construct
AETVEFWGCLGMGYDRTCWW

SEQ ID NO: 65 - protein, 20 aa, synthetic construct
AETVEFWTCWTVYGKSSCWD

SEQ ID NO: 66 - protein, 20 aa, synthetic construct
AETVEFYGCWWDAYGSTCTW

SEQ ID NO: 67 - protein, 20 aa, synthetic construct
AETVEFYGCDLWKGWTVCGY

SEQ ID NO: 68 - protein, 20 aa, synthetic construct
AETVEFRYCTHTQTYVQCQD

SEQ ID NO: 69 - protein, 20 aa, synthetic construct
AETVEFWGCNLDWYGWTCWW

SEQ ID NO: 70 - protein, 20 aa, synthetic construct
AETVEFWSCVVYGDNWTCSS

SEQ ID NO: 71 - protein, 20 aa, synthetic construct
AETVEFWSCQLGSGWWTCAW

SEQ ID NO: 72 - protein, 20 aa, synthetic construct
AETVEFWTCWETTEGYGCNW

SEQ ID NO: 73 - protein, 20 aa, synthetic construct
AETVEFWGCTGGAWEYTCWW

SEQ ID NO: 74 - protein, 20 aa, synthetic construct
AETVEFYGCSVTWGGYTCTW

SEQ ID NO: 75 - protein, 20 aa, synthetic construct
AETVEFYNCWYKMGWYVCWS

SEQ ID NO: 76 - protein, 17 aa, synthetic construct
AETVEFCRAYSNWFYIC

SEQ ID NO: 77 - protein, 618 aa, synthetic construct
MVSPRMSGLLSQTVILALIFLPQTRPAGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTSSCLSPRGPSSATTGCLVPGPGPCDGNPCANGGSCSETPRSFECTCPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRDCRERADPCAARPCAHGGRCYAHFSGLVCACAPGYMGARCEFPVHPDGASALPAAPPGLRPGDPQRYLLPPALGLLVAAGVAGAALLLVHVRRRGHSQDAGSRLLAGTPEPSVHALPDALNNLRTQEGSGDGPSSSVDWNRPEDVDPQGIYVISAPSIYAREVATPLFPPLHTGRAGQRQHLLFPYPSSILSVK

SEQ ID NO: 78 - skipped

SEQ ID NO: 79 - protein, 10 aa, synthetic construct
  VARIANT 2: note = X is Y, H, T, K, S, W, D, E, L, N, Q, or R
  VARIANT 4: note = X is D, N, or T
  VARIANT 5: note = X is W, A, S, N, R, or T
  VARIANT 6: note = X is D, E, G, Y, N, or S
  VARIANT 7: note = X is E, D, or N
  VARIANT 8: note = X is W, Y, or E
CXGXXXXXTC

SEQ ID NO: 80 - skipped

(19) United States
(12) Patent Application Publication
(10) Pub. No.: US 2025/0074943 A1
(43) Pub. Date: Mar. 6, 2025

(54) DLL3 BINDING PEPTIDES AND USES THEREOF

(71) Applicant: AMGEN INC., Thousand Oaks, CA (US)

(72) Inventors: Julie Bailis, Thousand Oaks, CA (US); Sylvie A.M. Tiso, Thousand, CA (US); Mark Salvati, Thousand Oaks, CA (US)

(21) Appl. No.: 18/719,056

(22) PCT Filed: Dec. 8, 2022
(86) PCT No.: PCT/US22/81212

§ 371 Date: Jun. 12, 2024

Related U.S. Application Data
(60) Provisional application No. 63/291,537, filed on Dec. 20, 2021.

(51) Int. Cl. C07K 7/08; A61K 38/00; A61K 47/60; A61K 49/00; A61K 51/06; A61K 51/08; C07K 7/06; G01N 33/574

(52) U.S. Cl. CPC C07K 7/08; A61K 47/60; A61K 49/0054; A61K 49/0056; A61K 51/065; A61K 51/088; C07K 7/06; G01N 33/57492; A61K 38/00

(57) ABSTRACT

The invention disclosed herein is directed to polypeptides that bind to DLL3 protein and uses thereof for the diagnosis and treatment of tumors/cancers.

### CROSS REFERENCE OF RELATED APPLICATION

This application claims the benefit of U.S. Provisional Application No. 63/291,537, filed on Dec. 20, 2021. The content of which is incorporated in its entirety by reference herein.

### SUBMISSION OF SEQUENCE LISTING ON ASCII TEXT FILE

The content of the following submission on ASCII text files is incorporated herein by reference in its entirety: a computer readable form (CRF) of the Sequence Listing (file name: A-2889-WO01-SEC_SeqListing.xml, date created: Dec. 8, 2022, size: 73,728 byes).

### FIELD OF THE INVENTION

Invention disclosed herein relates to polypeptides that bind to Delta-like ligand 3 (DLL3) and uses thereof for the diagnosis and treatment of tumors or cancers.

### BACKGROUND OF THE INVENTION

Delta-like ligand 3 (DLL3) is a type 1 transmembrane protein and noncanonical Notch ligand. DLL3 is a promising target for the development of therapies due to its high expression on the cell surface of neuroendocrine tumors, and minimal, mainly cytoplasmic localization in normal tissues. Owen et al., J Hematol Oncol., 12:61 (2019). Therapeutics against DLL3 have been developed in recent years for the treatment of DLL3-expressing tumors or cancers. See e.g., WO 2017/021349.

Labelled (e.g., radio labeled) small polypeptides are a class of pharmaceutical compounds used in the diagnosis and therapy of tumors or cancers. See e.g., Christine Rangger and Roland Haubner, Pharmaceuticals, 13(2):22 (2020). Compared to antibodies, these polypeptides are non-immunogenic and show fast diffusion and target localization. Additionally, peptides can be easily modified to improve metabolic stability and pharmacokinetics. Labelled small polypeptides also allow for noninvasive detection of tumor/cancer cells and monitoring of disease burden during or after treatment when used in tumor/cancer diagnosis.

No polypeptides that can bind to DLL3 and be used for cancer diagnosis and therapy have been reported. There is a need in the field for such compounds.

### SUMMARY OF THE INVENTION

The invention disclosed herein is directed to polypeptides that bind to human DLL3 protein and uses thereof for the diagnosis and treatment of tumors/cancers. It is demonstrated that the DLL3-binding peptides as described herein can be dimerized and/or functionalized for conjugating to radioisotopes as DLL3-targeted radiotracer to allow for noninvasive detection of DLL3-expressing tumor cells and monitoring of disease burden after treatment with DLL3-targeting molecules.

Accordingly, in one aspect, the invention provides a polypeptide comprising an amino acid sequence selected from C-X1-X2-X3-X4-X5-X6-X7-X8-C, wherein X1 is Y, H, T, K, S, W, D, E, L, N, Q, or R; X2 is G, W, Y, M, T, or V; X3 is D, N, Y, T, A, E, G, or S; X4 is W, A, E, S, V, Y, D, G, N, P, Q, R, or T; X5 is D, E, G, Y, N, W, K, R, or S; X6 is E, D, G, N, T, A, Q, or V; X7 is W, Y, V, E, or S; and X8 is T, G, A, or S (SEQ ID NO: 78); or alternatively, a polypeptide comprising the amino acid sequence CKWWGGAADEYTYSCGW (SEQ ID NO: 6).

In another aspect, the invention provides a polypeptide comprising an amino acid sequence selected from C-X1-X2-X3-X4-X5-X6-X7-X8-C, wherein X1 is Y, H, T, W, or N; X2 is G; X3 is D, N, or T; X4 is W, A, S, N, R, or T; X5 is D, E, G, Y, N, or S; X6 is E, D, or N; X7 is W, Y, or E; and X8 is T (SEQ ID NO: 79).

In some embodiments, the polypeptide comprises the amino acid sequence of any one of SEQ ID NOS: 1-23; any one of SEQ ID NOS: 1-7; any one of SEQ ID NOS: 39-61; or any one of SEQ ID NOS: 39-45.

In another aspect, the invention provides a polypeptide comprising the amino acid sequence of any one of SEQ ID NOS: 1-38. In some embodiments, the polypeptide comprises the amino acid sequence of any one of SEQ ID NOS: 39-76, preferably SEQ ID NOS: 39-61, and more preferably SEQ ID NOS: 39-45.

In some embodiments, the amino acid sequence described herein further comprises the amino acid residues AETVEF or AETVE at the N-terminal of the amino acid sequence. In some embodiments, the polypeptide described herein is modified at the N-terminus, the C-terminus, or both. In some embodiments, the amino acid residue at the N-terminus is acetylated. In other embodiments, the C-terminus of the polypeptide is aminated or amidated.

In some embodiments, the polypeptide described herein comprises a dimer of the amino acid sequence of SEQ ID NOS: 1-23, e.g., any one of the sequences for a homodimer or any two of the sequences for a heterodimer. In some embodiments, the polypeptide described herein comprises a dimer of the amino acid sequence of SEQ ID NOS; 1-7, or SEQ ID NOS; 24-38, or SEQ ID NOS: 39-76, or SEQ ID NOS: 39-61 or SEQ ID NOS: 39-45. In some embodiments, the dimer is a homodimer, for example, a homodimer of any of the sequences of SEQ ID NOS: 39-61 or SEQ ID NOS: 39-45. In some embodiments, the dimer comprises a first linker linking the two amino acid sequences.

In some embodiments, the polypeptide described herein further comprises a detectable agent. In some embodiments, the detectable agent comprises a fluorescent agent (e.g., Cy5 dye, an Alexa Fluor®647 dye, or a CF® 647 Dye) or a radioisotope (e.g., 67Ga, 99mTc, 111In, 68Ga, 64Cu, 44Sc, 86Y, 89Zr, 18F, 125I, 123I, 124I, or 203Pb; or 47Sc, 114mIn, 177Lu, 90Y, 212/213Bi, 212Pb, 225Ac, 186/188Re, 67Cu, 131I, 227Th, 211At, or 90Y). In some embodiments, the detectable agent is linked to the polypeptide via a second linker and/or a chelating agent. In some embodiments, the chelating agent is DOTA, TETA, DFO, NOTA, DTPA, HOPO, or Macropa.

In some embodiments, the first linker or the second linker independently comprises a peptide linker or a non-peptide linker. In some embodiments, the first or second linker comprises non-natural amino acids. In some embodiments, the first linker or the second linker independently comprises a poly(ethylene glycol) (PEG) linker (e.g., PEG3, PEG4, PEG6, and Bis-propargyl-PEG2). In some embodiments, the first linker further comprises hPra, Lys(N)3, Trioxatridecan-succinamic acid (Ttds), Gly-Gly or a combination thereof. In some embodiments, the second linker comprises a PEG linker. In some embodiments, the second liner further comprises a bicyclo[6.1.0]nonyne (BCN) group or a dibenzocyclooctyne (DBCO) group.

In some embodiments, the polypeptide described herein binds to DLL3 (e.g., human DLL3 expressed on the surface of a cell).

In another aspect, the invention provides a pharmaceutical composition comprising the polypeptides as described herein.

In another aspect, the invention provides a method of detecting DLL3 in a sample comprising contacting the polypeptide or the pharmaceutical composition described herein with the sample, and detecting DLL3 in the sample.

In some embodiments, the sample comprises a cell. In some embodiments, the cell is inside the body of a subject, and the method comprises administering the polypeptide or the pharmaceutical composition described herein to the subject and detecting DLL3 in the subject using an imaging technique (e.g., positron emission tomography (PET) scan). In some embodiments, the subject is a human with a DLL3-expressing tumor or cancer. In some embodiments, the tumor or cancer is small cell lung cancer (SCLC), non-small cell lung cancer (NSCLC), glioma, glioblastoma, melanoma, neuroendocrine prostate cancer, neuroendocrine pancreatic cancer, hepatoblastoma, large cell pulmonary neuroendocrine cancer, pancreatic neuroendocrine cancer, bladder neuroendocrine cancer, gastric neuroendocrine cancer, adrenal exocrine tumors, Merkel cell carcinoma, neuroblastoma, head and neck carcinoid or neuroendocrine cancer, head and neck paraganglioma, or cervical small cell neuroendocrine cancer.

In another aspect, the invention provides a method of treating a DLL3-expressing tumor or cancer, and the method comprises administering to a subject the polypeptide or the pharmaceutical composition described herein. In some embodiments, the subject is a human.

### BRIEF DESCRIPTION OF THE DRAWINGS

FIG. 1 shows binding of fluorescent labeled peptides, PepSP1215 (left) and PepSP1216 (right), to cells expressing hDLL3.

FIG. 2 shows huDLL3 expression of CHO cells engineered to express human DLL3 and the negative control untransfected CHO cells used in Example 4.

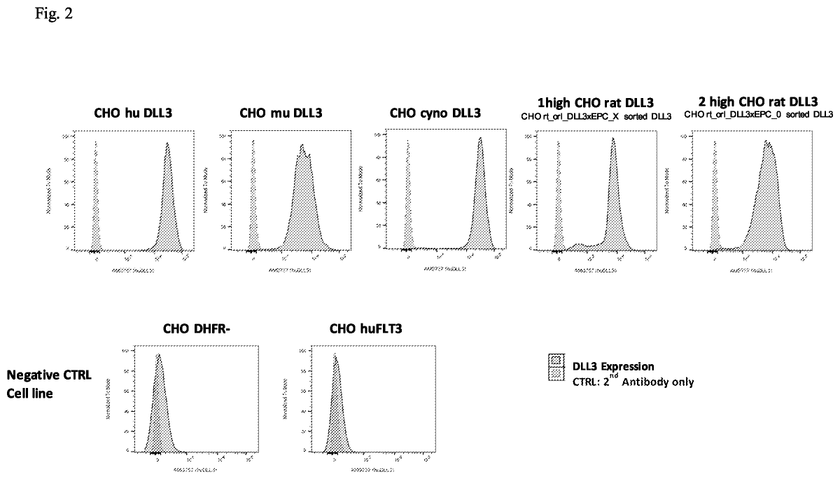

FIG. 3 shows binding of PepSP1462 labeled with biotin to CHO cells expressing or not expressing DLL3.

### DETAILED DESCRIPTION

The invention disclosed herein is directed to polypeptides that bind to human DLL3 protein and uses thereof for the diagnosis and treatment of tumors/cancers. It is demonstrated that the DLL3-binding peptides as described herein can be dimerized and/or functionalized for conjugating to radioisotopes as DLL3-targeted radiotracer to allow for noninvasive detection of DLL3-expressing tumor cells and monitoring of disease burden after treatment with various modalities (e.g., a DLL3-targeted immunotherapy). Accordingly, provided are polypeptides comprising an amino acid sequence of eight to thirteen (e.g., 8, 9, 10, 11, 12 or 13) amino acid residues flanked by two cysteine (Cys) residues. In certain embodiments, the number of amino acid residues between the two Cys residues is eight, nine or thirteen. In certain embodiments, the polypeptide further comprising additional amino acid residues at the N- or C-terminal or both of the amino acid sequence, for example, the polypeptide further comprising the amino acid residues AETVEF or AETVE at the N-terminal of the amino acid sequence. Polypeptides disclosed herein are identified by screening Cys constrained peptides phage libraries for binding to DLL3 (e.g., human DLL3).

In one aspect, the invention provides the polypeptide disclosed herein comprising an amino acid sequence selected from a) C-X1-X2-X3-X4-X5-X6-X7-X8-C(SEQ ID NO: 80), wherein X1 is W, T, Y, H, S, D, K, L, N, Q, E, R, or V; X2 is G, W, Y, L, V, T, E, H, M, or P; X3 is D, N, T, Y, G, W, A, E, K, L, M, S, or V; X4 is W, A, Y, G, S, E, Q, T, V, D, K, M, N, P, or R; X5 is G, Y, D, E, W, N, K, R, S, or T; X6 is G, E, D, N, W, T, A, K, Q, R, V, or Y; X7 is W, Y, V, S, E, P, R, T; and X8 is T, G, S, V, A, M, Q, or W.

In one aspect, the invention provides the polypeptide disclosed herein comprising an amino acid sequence selected from a) C-X1-X2-X3-X4-X5-X6-X7-X8-C, wherein X1 is Y, H, T, K, S, W, D, E, L, N, Q, or R; X2 is G, W, Y, M, T, or V; X3 is D, N, Y, T, A, E, G, or S; X4 is W, A, E, S, V, Y, D, G, N, P, Q, R, or T; X5 is D, E, G, Y, N, W, K, R, or S; X6 is E, D, G, N, T, A, Q, or V; X7 is W, Y, V, E, or S; and X8 is T, G, A, or S (SEQ ID NO: 78); or alternatively, b) a polypeptide comprising an amino acid sequence CKWWGGAADEYTYSCGW (SEQ ID NO: 6).

### Definitions

As used herein, the term “polypeptide” or “peptide” refers to a molecule of two or more amino acids joined by peptide bonds. Polypeptides described herein usually comprise 5 to 60 (e.g., 8 to 50) amino acids. Polypeptides may further form multimers such as dimers, trimers and higher oligomers, i.e., consisting of more than one polypeptide molecule. Polypeptide molecules forming such dimers, trimers etc. may be identical or non-identical. The corresponding higher order structures of such multimers are, consequently, termed homo- or heterodimers, homo- or heterotrimers etc. The terms “peptide” and “polypeptide” also refer to naturally modified peptides/polypeptides wherein the modification is affected e.g. by post-translational modifications like glycosylation, acetylation, phosphorylation and the like. A “peptide” or “polypeptide” when referred to herein may also be chemically modified such as modified at N- and/or C-terminal by, e.g., amidation, amination, or pegylated, or may be cyclic (e.g., via disulfate bonds). Such modifications are well known in the art and described herein below.

The term “amino acid” or “amino acid residue” typically refers to a building block of a protein such as an amino acid selected from the group consisting of: alanine (Ala or A); arginine (Arg or R); asparagine (Asn or N); aspartic acid (Asp or D); cysteine (Cys or C); glutamine (Gln or Q); glutamic acid (Glu or E); glycine (GIy or G); histidine (His or H); isoleucine (He or I): leucine (Leu or L); lysine (Lys or K); methionine (Met or M); phenylalanine (Phe or F); pro line (Pro or P); serine (Ser or S); threonine (Thr or T); tryptophan (Trp or W); tyrosine (Tyr or Y); and valine (Val or V), although modified, synthetic, or rare amino acids may be used as desired. Generally, amino acids can be grouped as having a nonpolar side chain (e.g., Ala, Cys, He, Leu, Met, Phe, Pro, Val); a negatively charged side chain (e.g., Asp, Glu); a positively charged sidechain (e.g., Arg, His, Lys); or an uncharged polar side chain (e.g., Asn, Cys, Gln, Gly, His, Met, Phe, Ser, Thr, Trp, and Tyr).

The terms “subject in need” or those “in need of treatment” includes those already with the disorder, as well as those in which the disorder is to be prevented. The subject in need or “patient” includes human and other mammalian subjects that receive either prophylactic or therapeutic treatment.

The term “treatment” refers to both therapeutic treatment and prophylactic or preventative measures. Treatment includes the application or administration of the polypeptide described herein to the body, an isolated tissue, or cell from a patient who has a disease/disorder, a symptom of a disease/disorder, or a predisposition toward a disease/disorder, with the purpose to cure, heal, alleviate, relieve, alter, remedy, ameliorate, improve, or affect the disease, the symptom of the disease, or the predisposition toward the disease.

The term “amelioration” as used herein refers to any improvement of the disease state of a patient having a tumor or cancer or a metastatic cancer as specified herein below, by the administration of a polypeptide according to the invention to a subject in need thereof. Such an improvement may also be seen as a slowing or stopping of the progression of the tumor or cancer or metastatic cancer of the patient.

The term “prevention” as used herein means the avoidance of the occurrence or re-occurrence of a patient having a tumor or cancer or a metastatic cancer as specified herein below, by the administration of a polypeptide according to the invention to a subject in need thereof.

The term “disease” refers to any condition that would benefit from treatment with the polypeptide or the pharmaceutic composition described herein. This includes chronic and acute disorders or diseases including those pathological conditions that predispose the mammal to the disease in question.

As used herein, the singular forms “a”, “an”, and “the” include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “about” or “approximately” as used herein means within ±20%, preferably within ±15%, more preferably within ±10%, and most preferably within ±5% of a given value or range.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim.

In each instance herein any of the terms “comprising”, “consisting essentially of” and “consisting of” may be replaced with either of the other two terms.

### DLL3 Binding Polypeptides

DLL3 is a non-canonical Notch ligand, functioning in a cell autonomous manner to inhibit Notch signaling, binding to Notch in cis, thus blocking cell to cell interactions and internalization of Notch in the target cell, a hallmark of canonical Notch signaling. The primary role for DLL3 is in somitogenesis during embryonic development. Mice with DLL3 knocked out show segmental defects in the axial skeleton and cranial and neuronal development. Somatic patterning defects are also seen in humans with certain germline DLL3 mutations, resulting in a condition called spondylocostal dysostosis (Bulman M. P. et al. Nature Genetics 24, 438-441 (2000)).

DLL3 is a promising target for the development of therapies due to its high expression on the cell surface of neuroendocrine tumors/cancer, and minimal, mainly cytoplasmic localization in normal tissues (Owen et al., J Hematol Oncol., 12:61 (2019)). Neuroendocrine tumors/cancer typically begin in neuroendocrine cells and can occur in organs such as the lungs, appendix, small intestine, rectum and pancreas.

Provided herein are polypeptides that bind to human DLL3 protein (e.g., SEQ ID NO: 77) and uses thereof for the diagnosis and treatment of tumors/cancers such as neuroendocrine tumors/cancers. In one aspect, the invention provides the polypeptide disclosed herein comprising an amino acid sequence selected from a) C-X1-X2-X3-X4-X5-X6-X7-X8-C, wherein X1 is Y, H, T, K, S, W, D, E, L, N, Q, or R; X2 is G, W, Y, M, T, or V; X3 is D, N, Y, T, A, E, G, or S; X4 is W, A, E, S, V, Y, D, G, N, P, Q, R, or T; X5 is D, E, G, Y, N, W, K, R, or S; X6 is E, D, G, N, T, A, Q, or V; X7 is W, Y, V, E, or S; and X8 is T, G, A, or S (SEQ ID NO: 78); or alternatively, b) a polypeptide comprising an amino acid sequence CKWWGGAADEYTYSCGW (SEQ ID NO: 6).

In another aspect, the invention provides a polypeptide comprising an amino acid sequence selected from C-X1-X2-X3-X4-X5-X6-X7-X8-C, wherein X1 is Y, H, T, W, or N; X2 is G; X3 is D, N, or T; X4 is W, A, S, N, R, or T; X5 is D, E, G, Y, N, or S; X6 is E, D, or N; X7 is W, Y, or E; and X8 is T (SEQ ID NO: 79).

In some embodiments, the polypeptide comprises the amino acid sequence of any one of any one of SEQ ID NOS: 1-38, any one of SEQ ID NOS: 1-23; any one of SEQ ID NOS: 1-7; any one of SEQ ID NOS: 39-61; or any one of SEQ ID NOS: 39-45, as depicted in Table 1 below. In some embodiments, the polypeptides described herein comprise the amino acid residues AETVEF or AETVE at the N-terminus of the amino acid sequence, for example, as depicted in Table 1. In some embodiments, the polypeptide comprises the amino acid sequence of any one of SEQ ID NOS: 39-76, preferably SEQ ID NOS: 39-61, or more preferably SEQ ID NOS: 39-45, as depicted in Table 1.

TABLE 1

| SEQ ID | |
|---|---|
| No. | Sequence |
| 1 | WWCNGNSENWTCTW |
| 2 | WGCTGTWGNETCWW |
| 3 | WDCWGNTDDWTCTW |
| 4 | YGCYGDRNDYTCTW |
| 5 | LGCHGDNSEWTCVW |
| 6 | WSCKWWGGAADEYTYSCGW |
| 7 | YGCTGDAYEYTCTW |
| 8 | WTCYWTEGVSGCRW |
| 9 | YWRCDWYEETVACGT |
| 10 | WSCHYSYWTVSCGT |
| 11 | YGCSMDWRGVTCAW |
| 12 | DYCLWAPDAWGCON |
| 13 | RGCTGNVYDWTCVW |
| 14 | WTCHGNADEWTCSW |
| 15 | DGCKGDWYEWTCWW |
| 16 | YGCRGGDGEWTCVW |
| 17 | DYCYGDVEEWTCVY |
| 18 | YGCETYSGGWTCLW |
| 19 | YGCKWDWKGWTCAW |
| 20 | YGCSVNADGWTCSW |
| 21 | YGCYGEQEQWTCLW |
| 22 | YGCQYYGWDYTCAW |
| 23 | YGCWGNYNDYTCSW |
| 24 | ETCWYLYGREWCVG |
| 25 | MRCYPWWYGPMCVR |
| 26 | WGCLGMGYDRTCWW |
| 27 | WTCWTVYGKSSCWD |
| 28 | YGCWWDAYGSTCTW |
| 29 | YGCDLWKGWTVCGY |
| 30 | RYCTHTQTYVQCQD |
| 31 | WGCNLDWYGWTCWW |
| 32 | WSCVVYGDNWTCSS |
| 33 | WSCQLGSGWWTCAW |
| 34 | WTCWETTEGYGCNW |
| 35 | WGCTGGAWEYTCWW |
| 36 | YGCSVTWGGYTCTW |
| 37 | YNCWYKMGWYVCWS |
| 38 | CRAYSNWFYIC |
| 39 | AETVEFWWCNGNSENWTCTW |
| 40 | AETVEFWGCTGTWGNETCWW |
| 41 | AETVEFWDCWGNTDDWTCTW |
| 42 | AETVEFYGCYGDRNDYTCTW |
| 43 | AETVEFLGCHGDNSEWTCVW |
| 44 | AETVEFWSCKWWGGAADEYTYSCGW |
| 45 | AETVEFYGCTGDAYEYTCTW |
| 46 | AETVEFWTCYWTEGVSGCRW |
| 47 | AETVEYWRCDWYEETVACGT |
| 48 | AETVEFWSCHYSYWTVSCGT |
| 49 | AETVEFYGCSMDWRGVTCAW |
| 50 | AETVEFDYCLWAPDAWGCON |
| 51 | AETVEFRGCTGNVYDWTCVW |
| 52 | AETVEFWTCHGNADEWTCSW |
| 53 | AETVEFDGCKGDWYEWTCWW |
| 54 | AETVEFYGCRGGDGEWTCVW |
| 55 | AETVEFDYCYGDVEEWTCVY |
| 56 | AETVEFYGCETYSGGWTCLW |
| 57 | AETVEFYGCKWDWKGWTCAW |
| 58 | AETVEFYGCSVNADGWTCSW |
| 59 | AETVEFYGCYGEQEQWTCLW |
| 60 | AETVEFYGCQYYGWDYTCAW |
| 61 | AETVEFYGCWGNYNDYTCSW |
| 62 | AETVEFETCWYLYGREWCVG |
| 63 | AETVEFMRCYPWWYGPMCVR |
| 64 | AETVEFWGCLGMGYDRTCWW |
| 65 | AETVEFWTCWTVYGKSSCWD |
| 66 | AETVEFYGCWWDAYGSTCTW |
| 67 | AETVEFYGCDLWKGWTVCGY |
| 68 | AETVEFRYCTHTQTYVQCQD |
| 69 | AETVEFWGCNLDWYGWTCWW |
| 70 | AETVEFWSCVVYGDNWTCSS |
| 71 | AETVEFWSCQLGSGWWTCAW |
| 72 | AETVEFWTCWETTEGYGCNW |
| 73 | AETVEFWGCTGGAWEYTCWW |
| 74 | AETVEFYGCSVTWGGYTCTW |
| 75 | AETVEFYNCWYKMGWYVCWS |
| 76 | AETVEFCRAYSNWFYIC |

In some embodiments, the DLL3 binding polypeptides described herein are modified at the N-terminus, the C-terminus, or both, which can improve the stability and/or metabolism of the peptide in vivo. In some embodiments, the amino acid residue at the N-terminus is acetylated. In some embodiments, the C-terminus of the polypeptide is modified, for example, aminated or amidated (e.g., —NH₂, —CONHR or —CONH₂). In some embodiments, the amino acid residue at the N-terminus is acetylated and the C-terminus of the polypeptide is aminated or amidated.

In some embodiments, the polypeptides described herein comprise a dimer of the amino acid sequences listed in Table 1. In some embodiments, the dimer is a homodimer (e.g., a polypeptide comprising a pair of a particular amino acid sequence listed in Table 1), or a heterodimer (e.g., a polypeptide comprising two different amino acid sequences listed in Table 1). In some embodiments, the polypeptide comprises a homodimer of any of SEQ ID NOS: 1-38, or any one of SEQ ID NOS: 39-76, or any of SEQ ID NOS: 39-61, or any one of SEQ ID NOS: 39-45.

In some embodiments, the polypeptide can be modified to improve target binding (e.g., pharmacokinetic properties), stability (e.g., resisting peptidase digestion), and metabolism (e.g., reduction in kidney retention, or hepatobiliary metabolism and clearance). For example, cyclization and modifications at N- and/or C-terminus of peptide can protect peptides from exopeptidases and/or endopeptidase. Another strategy for stability improvement towards endopeptidases is to use d-amino acids or unnatural amino acids such as naphthylalanine, phenylglycine, norleucine, and cyclohexylalanine. Modifications at N- and/or C-terminus (e.g., forming an amide at C-terminus such as —CONH₂ or —CONHR) of peptide may also be needed to link a detectable agent via a non-peptide linker such as a PEG linker comprising a DBCO (dibenzocyclooctyne) group or a BCN (bicyclo[6.1.0]nonyne) group.

In some embodiments, the dimer comprises a first linker linking the two amino acid sequences.

In some embodiments, the polypeptides described herein further comprise a detectable agent. In some embodiments, the detectable agent is linked to the polypeptide via a second linker and/or a chelating agent. For example, the polypeptides described herein can have the configuration of X-Y-Z, wherein X is a detectable agent (e.g., a radioisotope or a fluorescent agent described herein) with or without a chelator, Y is a linker (e.g., a second linker), and Z is the polypeptide (e.g., a polypeptide comprising a dimer of the amino acid sequences listed in Table 1 linked by a first linker).

In various embodiments, the first and second linker independently can be a peptide linker or a non-peptide linker. In some embodiments, the first and/or second linker each independently comprises a peptide linker such as short peptides consist of a few (e.g., 2-6) naturally and/or non-naturally occurring amino acids. Naturally occurring residues are divided into groups based on common side-chain properties. Exemplary groups include: (1) hydrophobic: norleucine, Met, Ala, Val, Leu, Ile; (2) neutral hydrophilic: Cys, Ser, Thr; (3) acidic: Asp, Glu; (4) basic: Asn, His, Lys, Arg; (5) residues that influence chain orientation: Gly, Pro; and (6) aromatic: Trp, Tyr, Phe. Examples of non-natural amino acids include, but are not limited to D-amino acids, homo amino acids, beta-homo amino acids, N-methyl amino acids, alpha-methyl amino acids. In some embodiment, the peptide linker comprises or consists of a lysine residue and/or two glycine residues (Gly-Gly).

In some embodiments, the first and/or second linker each independently comprises a non-peptide linker. Examples of non-peptide linkers include, but are not limited to, poly(ethylene glycol)(PEG) linkers, beta-alanine, 4-aminobutyric acid (GABA), (2-aminoethoxy) acetic acid (AEA), 5-aminovaleric acid (Ava), 6-aminohexanoic acid (Ahx), Trioxatridecan-succinamic acid (Ttds), fluorenylmethoxycarbonyl (Fmoc)-Lys(N3)-OH, Lys(N3), homopropargylglycine, and Fmoc-HPra-OH. These non-peptide linkers are known in the art and commercially available from companies such as Pepscan and BroadPharm.

PEG linkers include a group of compounds that are useful for various purposes (e.g., biolabeling). These linkers can be generally classified as monodispersed or poly-dispersed. A monodispersed PEG linker has an exact number of PEG units with a specific chemical structure and a precise molecular weight. In contrast, a poly-dispersed PEG (also known as Polymer PEG or PolyPEG) is a polymer with an averaged molecular weight. PEG linkers can also contain various functional groups such as Azide, Amine, Alkyne, DBCO (Dibenzocyclooctyne), BCN, TCO (trans-cycloctene), NHS ester, maleimide. Exemplary PEG linkers useful for the polypeptides disclosed herein include PEG linkers and PEG linkers containing various functional groups such as PEG2, PEG3, PEG4, PEG6, Bis-PEG (e.g., Bis-PEG18, Bis-PEG16, Bis-PEG14), Bis-propargyl-PEG (e.g., Bis-propargyl-PEG6, Bis-propargyl-PEG14 and Bis-propargyl-PEG18), Bis-PEG-NHS, BCN-PEG (e.g., PEG3-BCN), DBCO-PEG (e.g., PEG4-DBCO), and PEG-NHS-ester. These PEG linkers are commercially available from companies such as Pepscan, BroadPharm and JenKem Technology USA.

In some embodiments, the first linker comprises a peptide linker, a non-peptide linker or a combination thereof. For example, in some embodiments, the first linker comprises one or more of a PEG linker (e.g., PEG2, PEG3, PEG4, PEG6), a PEG linker with a functional group (e.g., PEG azide), gly-gly, Ttds, Fmoc-Lys(N3)-OH, homopropargylglycine, and Fmoc-HPra-OH. In some embodiments, the second linker comprises one or more of a PEG linker (e.g., PEG2, PEG3, PEG4, PEG6), a Bis-PEG linker (e.g., Bis-PEG16, Bis-PEG18), and a PEG linker with a functional group (e.g., BCN-PEG, DBCO-PEG). In some embodiments, the first liner comprises one or more of a PEG linker (e.g., PEG2, PEG3, PEG4, PEG6), a PEG linker with a functional group (e.g., PEG azide), gly-gly, Ttds, Fmoc-Lys(N3)-OH, homopropargylglycine, and Fmoc-HPra-OH, and the second linker comprises one or more of a PEG linker (e.g., PEG2, PEG3, PEG4, PEG6), a Bis-PEG linker (e.g., Bis-PEG16, Bis-PEG18), and a PEG linker with a functional group (e.g., BCN-PEG, DBCO-PEG).

The detectable agent can be a radioisotope or a fluorescent agent. In some embodiments, the detectable agent is a radioisotope. Examples of a radioisotope include, but are not limited to, 11C, 18F, 44Sc, 47Sc, 51Cr, 52mMn, 58Co, 52Fe, 56Ni, 57Ni, 62Cu, 64Cu, 67Cu, 66Ga, 68Ga, 67Ga, 72As, 77As, 75Br, 76Br, 77Br, 82Br, 86Y, 89Zr, 90Y, 94mTc, 99mTc, 97Ru, 105Rh, 109Pd, 111 Ag, 110mIn, 111In, 113mIn, 114mIn, 120I, 123I, 124I, 125I, 131I, 117mSn, 121Sn, 127Te, 142Pr, 143Pr, 149Pm, 151Pm, 149Tb, 153Sm, 157Gd, 161Tb, 166Ho, 165Dy, 169Er, 169Yb, 175Yb, 172Tm, 177Lu, 186Re, 188Re, 191Pt, 197Hg, 198Au, 199Au, 201T1, 203Pb, 211At, 212Bi, 213Bi, 212Pb, 225Ac, 227Th, and A118F2+. In some embodiments, the radioisotope is selected from 18F, 68Ga, 67Ga, 64Cu, 89Zr, 90Y, 99mTc, 11In, 123I, 124I, 131I, 177Lu, and A118F2+. In some embodiments, the radioisotope is selected from 64Cu, 67Ga, 68Ga, 99mTc, 11In, 123I, 131I, 90Y, 177Lu, 212Bi, 225Ac, and A118F2+.

Different radioisotopes can emit different particles such as alpha, beta or gamma particles, which can be used for imaging and/or therapeutic purpose. Typically, radioisotopes emitting gamma rays are used for imaging purposes (e.g., diagnostic imaging), while radioisotopes emitting alpha or beta rays are used for therapeutic purposes. Exemplary techniques for imaging purposes include single photon emission computed tomography (SPECT) and positron emission tomography (PET).

In some embodiments, the radioisotope is selected from 67Ga. 99mTc, 11In, 177Lu, 68Ga, 64Cu, 44Sc, 86Y, 89Zr, 18F, 125I, 123I, 124I, and 203Pb. In some embodiments, the radioisotope is 68Ga or 18F. In some preferred embodiments, the detectable agent is 18F. These exemplary radioisotopes can be used for imaging purpose.

In some embodiments, the radioisotope is selected from 47Sc, 114mIn, 177Lu, 90Y, 212/213Bi, 212Pb, 225Ac, 186/188Re, 67Cu, 131I, 227Th, 211At, and 90Y. In some embodiments, the radioisotope is selected from the group consisting of 177Lu, 225Ac, 67Cu, and 212/213Bi. These exemplary radioisotopes can be used for therapeutic purpose.

In some embodiments, the detectable agent is a radioisotope (e.g., a metal radioisotope) complexed with a chelator (also referred to as a chelating agent). The radioisotope/chelator complex can reduce radiation loss and/or hydrolysis of radioisotope and enable it to be delivered to a desirable/intended site in vivo. In some embodiments, the chelator is a bifunctional chelator, e.g., with one function/site that can bind to the radioisotope and the other function/site that can bind to the polypeptide directly or indirectly. Examples of a chelator include, but are not limited to, NODASA, NODAGA, TETA, TRITA, TRAP, DTPA, CHX-DTPA EDTA, CDTA, CPTA, DOTP, DOTPI, EGTA, HBED, TTHA, DTPA, DOTA, DOTAGA, NOTA, HP-DOA3, CBTE2a, TE2A, TMT, DPDP, HYNIC, DFO, HEDTA, NOPO.MAG3, NCS-MP-NODA, NH2-MPAA-NODA, DOTA, NODA, TRAP, DOTPI, DOTP, NOPO and TETA of DOTAGA, NOTA, DTPA, CHX-DTPA, NODA and functionalization thereof. In some embodiments, the chelator is DOTA, TETA, NOTA, NETA, TACN-TM, DTPA, 1B4M-DTPA, CHX-A00-DTPA, TRAP (PRP9) NOPO, H2dedpa, H4octapa, H2azapa, and H5decapa, HBED, SHBED, BPCA, CP256, PCTA, Desferrioxamine (DFO), HEHA, and PEPA. Chelators appropriate for radioisotopes are known in the art and can be readily selected by a person of ordinary skill in the art, see e.g., Christine Rangger and Roland Haubner, Pharmaceuticals 2020, 13, 22; doi:10.3390/ph13020022; Eric W. Price and Chris Orvig, Chem. Soc. Rev., 2014, 43, 260-290. In some embodiments, the chelator is DOTA (e.g., for 11In, 177Lu, or 213Bi), TETA (e.g., for 64Cu/67Cu), DFO (e.g., for 89Zr), NOTA (e.g., for 68Ga or 64Cu/67Cu), DTPA (e.g., for 111In), HOPO (e.g., for 89Zr or 227Tr), or Macropa (e.g., for 225Ac).

In some embodiments, the detectable agent is a fluorescent agent. fluorescence agents are known in the art and are commercially available. Examples of fluorescent agents include, but are not limited to, Cy3 dye, Cy5 dye, Fluoresceinisothiocyanate (FITC), Anthranilyl, 2-Aminobenzoyl (Abz), 5-Carboxyfluorescein (5-FAM), 6-Carboxyfluorescein (6-FAM), Carboxytetramethyl rhodamine (TAMRA), 5-(Dimethylamino) naphthalene-1-sulfonyl (Dansyl), 5-[(2-Aminoethyl)amino]naphthalene-1-sulfonic acid (EDANS), and 7-Methoxycoumarinyl-4-acetyl (Mca). Fluorescence labeled peptides can be prepared by either modifying isolated peptides or by incorporating the label during solid-phase synthesis using methods known in the art.

The polypeptide described herein can be made by methods known in the art such as solid phase peptide synthesis. See e.g., P. Lloyd-Williams, F. Albericio and E. Girald; Chemical Approaches to the Synthesis of Peptides and Proteins, CRC Press, 1997.

The polypeptides can be labeled directly by reacting the radioisotope with the polypeptide for direct labeling using a single step. See, e.g., Williams J. et al., Bioconjugate Chem., 32(7): 1242-1254 (2021). In some embodiments, a carrier molecule can be labeled with the radioisotope, purified (if needed) and then reacted with the polypeptide to produce the labeled polypeptide in a two-step reaction. See, e.g., Yu S., Biomed Imaging Interv J., 2(4): e57 (October-December 2006). In some embodiments, the peptide is dimerized and/or labeled with a detectable agent (e.g., radioisotope or fluorescent agent) using “Click Chemistry” known in the art such as the Huisgen 1,3-dipolar cycloaddition of azides and terminal alkynes. See e.g., Hein, Christopher D. et al., Pharm Res. 2008 October; 25(10): 2216-2230. doi:10.1007/s11095-008-9616-1; Kolb, H. C. et al., Angew. Chem. Int. Ed. 2001, 40, 2004-2021. Click Chemistry refers to chemical reactions between pairs of reagents (click chemistry tools) to react with each other under mild condition and is effectively inert to naturally occurring functional groups such as the amine group. Reagents for click chemistry are known in the art and commercially available from companies such as BroadPharma and AlphaThera. In some embodiments, the peptide is dimerized and/or labeled with a detectable agent (e.g., radioisotope or fluorescent agent) using Scheme I, and/or Scheme II, and/or Scheme III disclosed herein (Example 3).

Polypeptides disclosed herein can bind to DLL3. In some embodiments, the polypeptides bind to human DLL3, e.g., human DLL3 expressed in a cell such as a recombinant cell expressing huDLL3 or a DLL3 expressing cancer cell in a patient. The amino acid sequence of human DLL3 is listed below (SEQ ID NO: 77). In some embodiments, the polypeptides comprise any of the amino acid sequences listed in Table 1, in some embodiments, the polypeptides comprise a dimer of any of the amino acid sequences listed in Table 1, in some embodiments, the polypeptides are modified at the N-terminal, the C-terminal or both ends of the peptide. In some embodiments, the dimer is a homodimer.

Human DLL3
(SEQ ID NO: 77)
MVSPRMSGLLSQTVILALIFLPQTRPAGVFELQIHSFGPGPGPGAPRSP
CSARLPCRLFFRVCLKPGLSEEAAESPCALGAALSARGPVYTEQPGAPA
PDLPLPDGLLQVPFRDAWPGTFSFIIETWREELGDQIGGPAWSLLARVA
GRRRLAAGGPWARDIQRAGAWELRFSYRARCEPPAVGTACTRLCRPRSA
PSRCGPGLRPCAPLEDECEAPLVCRAGCSPEHGFCEQPGECRCLEGWTG
PLCTVPVSTSSCLSPRGPSSATTGCLVPGPGPCDGNPCANGGSCSETPR
SFECTCPRGFYGLRCEVSGVTCADGPCFNGGLCVGGADPDSAYICHCPP
GFQGSNCEKRVDRCSLQPCRNGGLCLDLGHALRCRCRAGFAGPRCEHDL
DDCAGRACANGGTCVEGGGAHRCSCALGFGGRDCRERADPCAARPCAHG
GRCYAHFSGLVCACAPGYMGARCEFPVHPDGASALPAAPPGLRPGDPQR
YLLPPALGLLVAAGVAGAALLLVHVRRRGHSQDAGSRLLAGTPEPSVHA
LPDALNNLRTQEGSGDGPSSSVDWNRPEDVDPQGIYVISAPSIYAREVA
TPLFPPLHTGRAGQRQHLLFPYPSSILSVK

### Compositions and Formulations

The invention further provides a pharmaceutical composition comprising a polypeptide as described herein.

As used herein, the term “pharmaceutical composition” relates to a composition which is suitable for administration to a subject or a patient, preferably a human subject or a patient. The particularly preferred pharmaceutical composition of this invention comprises one or a plurality of the polypeptide of the invention. Preferably, the pharmaceutical composition further comprises suitable formulations of one or more (pharmaceutically effective) carriers, stabilizers, excipients, diluents, solubilizers, surfactants, emulsifiers, preservatives and/or adjuvants. Acceptable constituents of the pharmaceutical composition are preferably nontoxic to recipients at the dosages and concentrations employed. Pharmaceutical compositions of the invention include, but are not limited to, liquid, frozen, and lyophilized compositions.

Excipients that can be used in the pharmaceutical composition include gentisic acid, maleic acid, beta-cyclodextrin, alpha-cyclodextrin, ascorbic acid, thioglycerol, glutathione, tartaric acid, niacinamide, ascorbic acid, FeCl3, glutamic acid, methylene diphosphonic acid, beta-hydroxypropyl cyclodextrin, xanthine, aspartic acid, calcium chloride, mannitol, calcium gluconate, sodium succinate, boric acid, sodium carbonate, sodium chloride, or combinations thereof.

In some embodiments, the pH of the pharmaceutical composition is in the range of from about pH 4.5 to about pH 8.0, e.g., from about pH 5.0 to about pH 7.5, which can be achieved by using one or more buffers such as those known in the art. Exemplary buffers include acetate buffer, phosphate buffer, and citrate buffer.

In some embodiments, the polypeptide comprised in the pharmaceutical composition is a polypeptide comprising a detectable agent such as a radioisotope or a fluorescent agent. In some embodiments, the polypeptide comprises a radioisotope such as 67Ga, 99mTc, 11In, 68Ga, 64Cu, 44Sc, 86Y, 89Zr, 18F, 125I, 123I, 124I, or 203Pb. In some embodiments, the polypeptide comprises 68Ga or 18F. In some embodiments, the polypeptide comprises a radioisotope such as 47Sc, 114mIn, 177Lu, 90Y, 212/213Bi, 212Pb, 225Ac, 186/188Re, 67Cu, 131I, 227Th, 211At, or 90Y.

In embodiments wherein the pharmaceutical composition comprises a polypeptide that comprises a radioisotope, the pharmaceutical composition can comprise one or more agents such as N-tert-Butyl-α-phenylnitrone (PBN), ethanol, Sodium Ascorbate, and gentisic acid. Inclusion of such agents can stabilize and protect the radioisotope. Such agents can be used at an amount that is nontoxic to recipients at the dosages and concentrations employed.

In some embodiments, the pharmaceutical composition is a liquid composition. In some embodiments, the pharmaceutical composition is a solid composition such as a lyophilized composition. In some embodiments, the pharmaceutical composition (e.g., a liquid composition or a reconstituted lyophilized composition) is suitable for intravenous administration.

### Diagnostics and Treatments

Polypeptides disclosed herein such as those comprising a detectable agent can be used for detecting DLL3 (e.g., human DLL3) in a sample. The sample can be a cell expressing DLL3 such as a recombinant cell expressing DLL3 or a DLL3-expressing tumor or cancer cell. Thus, disclosed herein is a method of detecting DLL3 in a sample comprising contacting a polypeptide comprising a detectable agent as described above or a pharmaceutical composition comprising the polypeptide with the sample, and detecting DLL3 in the sample.

In some embodiments, the polypeptide comprises any one of SEQ ID NOS: 1-23; any one of SEQ ID NOS: 1-7; any one of SEQ ID NOS: 39-76; any one of SEQ ID NOD: 39-61, or any one of SEQ ID NOS: 39-45, listed in Table 1, and a detectable agent. In some embodiments, the polypeptide comprises a dimer of the amino acid sequence of SEQ ID NOS: 1-38 or of SEQ ID NOS: 39-76, and a detectable agent. In some embodiments, the dimer is a homodimer, e.g., the polypeptide comprises a homodimer of any one of SEQ ID NOS: 1-38, or a homodimer of any one of SEQ ID NOS: 39-76. In some embodiments, the polypeptide comprises a homodimer of any one of SEQ ID NOS: 39-61. In some embodiments, the dimer is a heterodimer, e.g., the polypeptide comprises any two different sequences of SEQ ID NOS: 1-38 or any two different sequences of SEQ ID NOS: 39-76. In some embodiments, the homodimer or heterodimer comprises a first linker linking the two amino acid sequences. Linkers suitable for use as the first linker are described above.

In some embodiments, the detectable agent is a radioisotope such as 67Ga, 99mTc, 111In, 68Ga, 64Cu, 44Sc, 86Y, 89Zr, 18F, 125I, 123I, 124I, or 203Pb. In some embodiments, the radioisotope is 67Ga or 18F. In some embodiments, the radioisotope is 18F. In some embodiments, the radioisotope is linked to the polypeptide via a second linker, a chelating agent, or a combination thereof. Suitable chelating agents and linkers that can be used as a second linker are described above.

In some embodiments, the detectable agent is a fluorescent agent such as a Cy3 agent, a Cy5 dye, Fluoresceinisothiocyanate (FITC), Anthranilyl, 2-Aminobenzoyl (Abz), 5-Carboxyfluorescein (5-FAM), 6-Carboxyfluorescein (6-FAM), Carboxytetramethyl rhodamine (TAMRA), 5-(Dimethylamino) naphthalene-1-sulfonyl (Dansyl), 5-[(2-Aminoethyl)amino]naphthalene-1-sulfonic acid (EDANS), or 7-Methoxycoumarinyl-4-acetyl (Mca). In some embodiments, the fluorescent agent is linked to the polypeptide via a second linker, as described above.

In some embodiments, the sample comprises a cell expressing DLL3 such as human DLL3. In some embodiments, the cell is a recombinant cell expressing human DLL3. In some embodiments, the cell is a DLL3-expressing tumor or cancer cell such as a cell obtained from a cancer patient.

Methods that can be used for detecting the polypeptide in the sample include those known in the art. For example, flow cytometry can be used when the detectable agent is a fluorescent agent, while imaging method such as PET or SPECT can be used when the detectable agent is a radioisotope.

In some embodiments, the cell is inside the body of a subject, and the method comprises administering the polypeptide or the pharmaceutical composition comprising the polypeptide to the subject and detecting DLL3 in the subject using an imaging technique. The polypeptide or the composition comprising the polypeptide can be administered to the subject by parenteral administration. In some embodiments, the polypeptide or composition thereof is administered by intravenous administration. In some embodiments, the subject is a human with a DLL3-expressing cancer or tumor such as a cancer of neuroendocrine origin. In some embodiments, the tumor or cancer is lung cancer such as small cell lung cancer (SCLC) or non-small cell lung cancer (NSCLC), glioma, glioblastoma, melanoma, prostate cancer such as neuroendocrine prostate cancer, neuroendocrine pancreatic cancer, hepatoblastoma, large cell pulmonary neuroendocrine cancer, pancreatic neuroendocrine cancer, bladder neuroendocrine cancer, gastric neuroendocrine cancer, adrenal exocrine tumors, Merkel cell carcinoma, neuroblastoma, head and neck carcinoid or neuroendocrine cancer, head and neck paraganglioma, or cervical small cell neuroendocrine cancer. In some embodiments, the tumor or cancer is prostate cancer (e.g., neuroendocrine prostate cancer) or lung cancer (e.g., small cell lung cancer). In some embodiments, the imaging technique is positron emission tomography.

Polypeptides disclosed herein such as those comprising a radioisotope can be used for treatment of tumor or cancer in a subject. Radioisotopes that are useful for cancer treatment include those emitting alpha or beta particles. In one aspect, disclosed herein also provides a method of treating a DLL3-expressing tumor or cancer disease comprising administering to a subject in need thereof the polypeptide that comprises a radioisotope described herein or the pharmaceutical composition comprising the polypeptide described herein.

In some embodiments, the polypeptide comprises any one of SEQ ID NOS: 1-23; any one of SEQ ID NOS: 1-7; any one of SEQ ID NOS: 39-76; any one of SEQ ID NOD: 39-61, or any one of SEQ ID NOS: 39-45, listed in Table 1, and a radioisotope. In some embodiments, the polypeptide comprises a dimer of the amino acid sequence of SEQ ID NOS: 1-38 or of SEQ ID NOS: 39-76, and a radioisotope. In some embodiments, the dimer is a homodimer, e.g., the polypeptide comprises a homodimer of any one of SEQ ID NOS: 1-38, or a homodimer of any one of SEQ ID NOS: 39-76. In some embodiments, the polypeptide comprises a homodimer of any one of SEQ ID NOS: 39-61. In some embodiments, the dimer is a heterodimer, e.g., the polypeptide comprises any two different sequences of SEQ ID NOS: 1-38 or any two different sequences of SEQ ID NOS: 39-76. In some embodiments, the homodimer or heterodimer comprises a first linker linking the two amino acid sequences. Linkers suitable for use as the first linker are described above.

In some embodiments, the radioisotope is 47Sc, 114mIn, 177Lu, 90Y, 212/213Bi, 212Pb, 225Ac, 186/188Re, 67Cu, 131I, 227Th, 211At, or 90Y. In some embodiments, the radioisotope is linked to the polypeptide via a second linker, a chelating agent or a combination thereof. In some embodiments, the radioisotope is linked to the polypeptide via a chelating agent. Chelating agents and linkers suitable for use as the second linker are described above.

In some embodiments, the subject is a human having a DLL3-expressing tumor or cancer such as a cancer of neuroendocrine origin. In some embodiments, the tumor or cancer is lung cancer such as SCLC or NSCLC, glioma, glioblastoma, melanoma, prostate cancer such as neuroendocrine prostate cancer, neuroendocrine pancreatic cancer, hepatoblastoma, large cell pulmonary neuroendocrine cancer, pancreatic neuroendocrine cancer, bladder neuroendocrine cancer, gastric neuroendocrine cancer, adrenal exocrine tumors, Merkel cell carcinoma, neuroblastoma, head and neck carcinoid or neuroendocrine cancer, head and neck paraganglioma, or cervical small cell neuroendocrine cancer. In some embodiments, the tumor or cancer is prostate cancer or lung cancer, in some embodiments, the tumor or cancer is neuroendocrine prostate cancer or small cell lung cancer.

In some embodiments, the polypeptide or the pharmaceutical composition comprising the polypeptide is administered to the subject (e.g., a human patient) via parenteral administration. In some embodiments, the polypeptide or the pharmaceutical composition comprising the polypeptide is administered to the subject via intravenous administration.

If the pharmaceutical composition has been lyophilized, the lyophilized material is first reconstituted in an appropriate liquid prior to administration. The lyophilized material may be reconstituted in, e.g., bacteriostatic water for injection (BWFI), physiological saline, phosphate buffered saline (PBS), or the same formulation the polypeptide had been in prior to lyophilization.

In a further embodiment, disclosed herein provides a kit comprising a polypeptide disclosed herein such as those comprising a detectable agent useful for diagnostic or therapeutic purpose. In some embodiments, the detectable agent is a radioisotope. In some embodiments, the kit further comprises an instruction (e.g. in the form of a leaflet or instruction manual) of how to use the polypeptide. The kit may also comprise means for administering the polypeptide or a pharmaceutical composition thereof such as a syringe, pump, infuser or the like, means for reconstituting the polypeptide and/or means for diluting the polypeptide.

In the context of the present invention, the term “kit” means two or more components—one of which corresponding to the polypeptide or the pharmaceutical composition of the invention—packaged together in a container, recipient or otherwise. A kit can hence be described as a set of products and/or utensils that are sufficient to achieve a certain goal, which can be marketed as a single unit.

All patents and other publications identified are expressly incorporated herein by reference in their entirety or in relevant part, as would be apparent from the context of the citation, for the purpose of describing and disclosing, for example, the methodologies described in such publications that might be used in connection with information described herein.

Inventions disclosed herein are further illustrated by the examples below.

### EXAMPLES

### Example 1. Selection and Screening of DLL3 Binding Peptides from Phage Libraries

The selections were carried out using two Cys constrained peptides phage libraries 9-CYS and 12-C8C displayed on pIII major coat protein of M13. Four different strategies of selection were carried out as shown in Table 2, performing the rounds of selection combining the use of DLL3 recombinant protein and three different stable transfected cell lines expressing human DLL3 on the surface at different levels.

TABLE 2

| Selection A | Selection B | Selection B1 (only 12C8C) | Selection C |
|---|---|---|---|
| R1: protein | R1: protein | R1: protein | R1: protein |
| R2: protein | R2: HEK293-DLL3 F8 | R2: HEK293-huDLL3 A6 | R2: CHO-huDLL3 |
| R3: protein | R3: HEK293-DLL3 F8 | R3: HEK293-huDLL3 A6 | R3: CHO-huDLL3 |
| | R4: HEK293-DLL3 F8 | R4: HEK293-huDLL3 A6 | R4: CHO-huDLL3 |
| Cell clone | DLL3 Receptor density | | |
| HEK293- | 3000 | Low | |
| huDLL3 F8 | | | |
| HEK293- | 127000 | High | |
| huDLL3 A6 | | | |
| CHO-huDLL3 | 13600 | Medium | |

The first round of selection is common to all four strategies where recombinant human DLL3 (rDLL3) was used. 10 μg rDLL3-Fc were incubated with Protein A Dynabeads (Life Technologies) to immobilize the protein on the beads. The beads were then washed twice with PBS buffer before incubation with pre-blocked phages. About 10¹¹ phage from the libraries were used in this round of selection. Briefly, phage particles were blocked in buffer at room temperature, then incubated with the immobilized protein. At the end of the incubation, beads were collected with a magnet and washed 5 times in PBS buffer. Bound phages were eluted using 100 mM triethylamine (TEA) and then used to infect TG1 bacteria. After 1 hr at 37° C., the bacteria were plated onto 2×TY/Amp/Glu plates and incubated at 30° C. o/n.

The next day, colonies were collected from the plates in 10 ml of culture medium and used to rescue the selected phages. The collected bacteria were inoculated into 50 ml of 2×TY/Amp/Glu, grown at 37° C. until OD 600 nm=0.5, and then infected with K07 helper phage at an MOI of approximately 10 (10 phage per cell). Infected bacteria were spun down, re-suspended into 2×TY/Amp/Kan and grown overnight at 25° C. The next day, the supernatant was clarified by centrifugation and then the infected bacteria were blocked in PBS with 4% milk.

Parental cell lines (CHO and HEK 293) and DLL3 overexpressing cell lines were detached, counted, and dispensed at 10⁷ cells/tube. The cells were blocked in PBS with 3% milk and incubated with the pre-blocked phages. The DLL3 overexpressing cells and the phages were incubated for one hour and unbound phages were removed by washing in PBS. The phages were then eluted by resuspending cells into 500 μl of 100 mM HCl for 10 min. The eluted phages were used to infect 10 ml of mid log culture of TG1. The bacteria were then plated onto 2×TY/Amp/Glu plates and incubated at 30° C. overnight. The process was repeated for two more times in 4 rounds of selection.

The pools of phages obtained after each round of selection were tested by Phage-ELISA for their binding to rDLL3-Fc and counter-screened on hIgG-Fc. Briefly, proteins were immobilized on NUNC-Maxisorp ELISA plates. Then the plates were washed and blocked. 100 μl of pre-blocked phages were added to the blocked plate and incubated for 1 hr at room temperature. Anti-M13 HRP conjugated mAb were added and the plates were read on a Multiskan Ascent (Thermo) Elisa reader at A370 nm after the addition of TMB (Sigma).

The pools of phages were also screened for their binding to cells overexpressing human DLL3 on their surface. Cells were detached, counted and re-suspended at 1.8×10⁶ cell/ml in FACS buffer containing 2% FBS. Pre-blocked phages were then added to the cells and incubated for 1 hr at room temperature. After wash, 1 μg of α-M13-FITC (clone MM05T, Sino Biologics) was added to the cells and incubated for 1 additional hour. Cells were then washed and re-suspended into FACS buffer. Fluorescence was acquired using a flow cytometer (FACS Canto/Becton Dickinson).

Single phage clones were picked after each round of selection and grown. Bacterial cultures were infected with helper phage at MOI of approximately 10 and incubated at 37° C. Infected bacteria were spun down, re-suspended and grown overnight at 25° C. The next day, the supernatants were clarified by centrifugation. An aliquot of the supernatant was used for the phage ELISA assay using the same method described above, or for FACS staining on DLL3 expressing cells.

Phage clones with signal higher than four-fold above the background were further tested for their binding to DLL3 on the surface of cells expressing DLL3 by cell ELISA. Briefly, parental cells and cells overexpressing DLL3 were plated on 96 well plate the day before the assay. The cells were then fixed and washed before the addition of pre-blocked phages as previously described. The cells were then washed and anti-M13-HRP was added. The signal was measured using a Multiskan Ascent (Thermo) ELISA reader at A370 nm. Phages that bind to DLL3 expressing cells were selected for sequencing of the displayed peptide.

The single phage clones that showed specific binding to the rDLL3-Fc were then tested for binding to DLL3 stably expressed on the surface of cells to identify clones that recognize the native form of the protein. All the phages that were able to recognize the cells were subjected to DNA sequencing of the displayed peptide.

In summary, 3400 single clones were analyzed. Among them, 1718 bind to the recombinant DLL3 protein and 387 of them also bind to the DLL3 protein on the surface of a cell line stably expressing DLL3.

Sequence analysis identified a total of 38 unique sequences, listed in Table 1 (SEQ ID NOS:39-76). Among them, 37 of which derived from the library 12-C8C and 1 from the library 9-CYS.

### Example 2. Synthesis, Modification and Characterization of DLL3 Binding Peptides

### Materials and Methods

Peptide synthesis: Peptide synthesis was performed by standard Fmoc stepwise solid phase synthesis (SPPS) using a Liberty Blue microwave synthesizer (CEM corp.). The synthesis was carried out using a Rink amide AM Resin Novabiochem 0.29 mmol/g on a 150 μmol scale. Each amino acid (0.2M in DMF) was acylated with an 8-fold excess using equimolar amounts of DIC (0.5M) and Oxyma (1 M) in DMF as activators. Amino acids were used with standard side chain protecting groups unless otherwise noted. Cysteines required for disulfide bridge were acylated on solid phase as Fmoc-Cys(Trt)-OH. Aspartic acid was coupled as Fmoc-Asp (OMpe)-OH to minimize aspartimide formation, and Fmoc deprotection, following incorporation of Asp-(OMpe)-OH, was carried out at room temperature with 20% piperidine in DMF. Single and double couplings were done under microwave irradiation at 90° C. for 2 min, with the exception of Fmoc-His(Trt)-OH (50° C.). Double acylation reactions were done for all Fmoc-Arg(Pbf)-OH and for the first three amino acids at the N-terminus (AET). The peptides were cleaved using a solution of 87.5% TFA, 5% H₂O, 2.5% TIS, 5% phenol for 1.5 h at room temperature and then precipitated with cold tert-butyl methyl ether. After centrifugation, the peptide pellets were washed with diethyl ether, dried, dissolved in 0.1% TFA in (1:1) H₂O/ACN, and lyophilized.

Analytical Characterization: Crude and purified peptides were analyzed by ultra-high performance liquid chromatography with UV and mass spectrometry detection (UPLC-UV-MS). Analyses were performed on a Waters Acquity UPLC system equipped with an analytical Waters BEH130 C4 (2.1×100 mm, 1.7 μm, at 45° C.) column. Detection was performed by UV absorbance at 214 nm. Mass analysis was performed on a Waters SQ Detector with electrospray ionization in positive ion detection mode and the scan range of the mass-to-charge ratio was 400-1800. Analyses were performed using linear gradient of binary mixtures of H₂O containing 0.1% TFA (A) and acetonitrile containing 0.1% TFA (B). The linear gradients of B used was 20% B-20% B (1 min) 20% B-60% B (4 min), 60% B-80% B (0.2 min); flow: 0.4 mL/min; Temp: 45° C.

N-terminal acetylation: At the end of the sequence assembly, the resin was acetylated using 10 molar equivalents of acetic anhydride in DMF. It is believed that the blocking of the N-terminal amino group reactivity of a polypeptide by acetylation can enable a straightforward synthetic strategy for linking a detectable agent (e.g., fluorescence of radiolabeled moieties) to the polypeptide.

C-terminal amidation or amination: C-terminal amidation or amination was done during solid phase synthesis using methods known in the art.

Cyclization: Cyclization is believed to restrict the conformation of peptide binders for improved activity towards a target. If needed, peptides were cyclized between the two cysteine groups via a disulfide bridge to form a cyclic structure and purified using the method described herein. The peptides were incubated in 10% DMSO, 90% Tris 0.1M pH8 (final peptide concentration of 1 mg/ml) overnight for disulfide formation. The reaction was monitored by UPLC analysis on a BEH130 C4 Acquity Waters column (2.1×100 mm, 1.7 μm) with a gradient of 20% B-20% B (1 min) 20% B-60% B (4 min), 60% B-80% B (0.2 min); flow: 0.4 mL/min; Temp: 45° C. The reaction was quenched with TFA and DMSO was added up to the total dissolution of the peptide before purification.

HPLC Purification: Reversed-phase HPLC of cyclized peptides was performed with a preparative HPLC Waters system using C4 (Waters DeltaPak 200×20 mm, 300 Å, 15 μm) column and appropriate linear gradients of increasing concentration of acetonitrile in water, 0.1% TFA (15% B-30% B in 20 min; 20% B-35% B in 20 min; 25% B-40% B in 20 min; flow rate of 80 mL/min). Fractions containing the desired product were combined and lyophilized. The characterization was performed using the linear gradient of acetonitrile in water: 20% B-20% B (1 min) 20% B-40% B (4 min), 40% B-80% B (0.2 min); 25% B-25% B (1 min) 25% B-45% B (4 min), 45% B-80% B (0.2 min); 30% B-30% B (1 min) 30% B-50% B (4 min), 50% B-80% B (0.2 min).

Sequences and structures of the exemplary cyclized peptides are shown in Table 3 below.

TABLE 3

| PepSP ID | Sequence & Structure* |
|---|---|
| PepSP1146 | AETVEFWGCTGTWGNETCWW-CONH2 |
| | 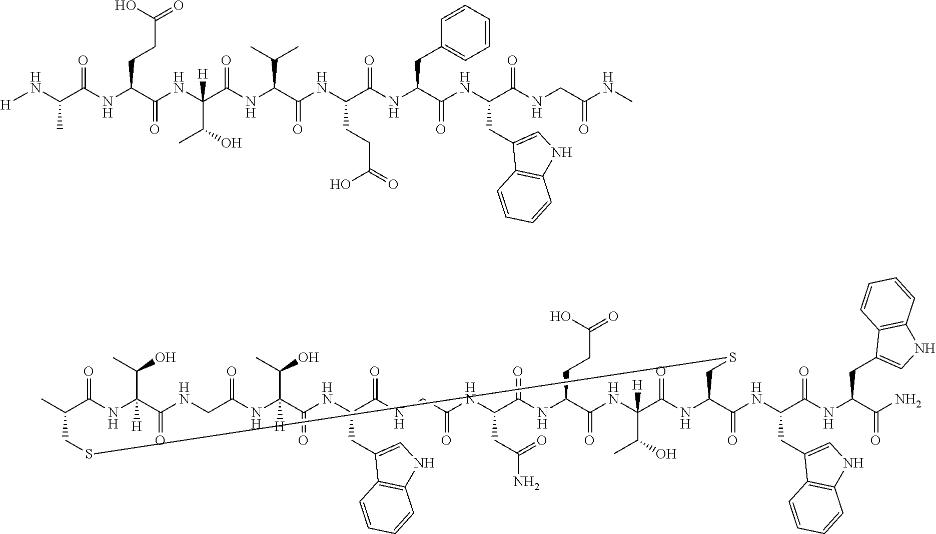 |
| PepSP1147 | AETVEFYGCTGDAYEYTCTW-CONH2 |
| | 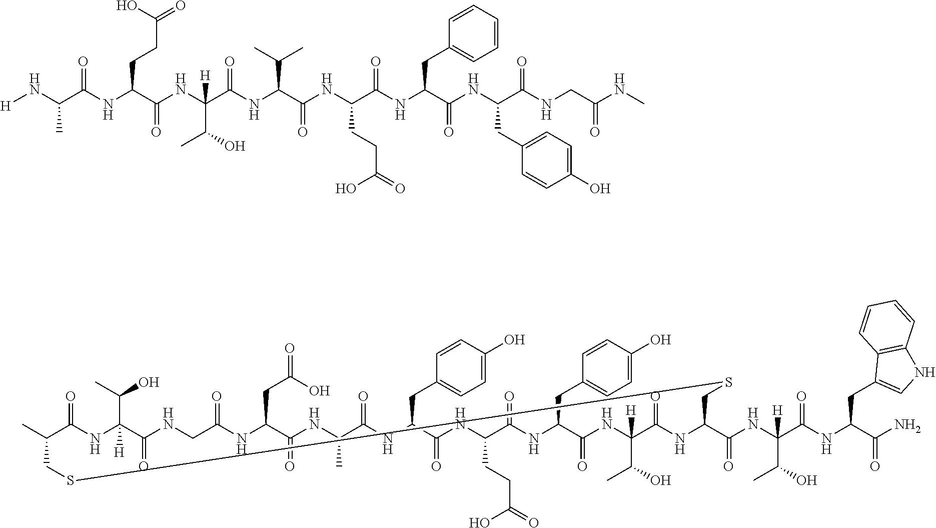 |
| PepSP1148 | AETVEFETCWYLYGREWCVG-CONH2 |
| | 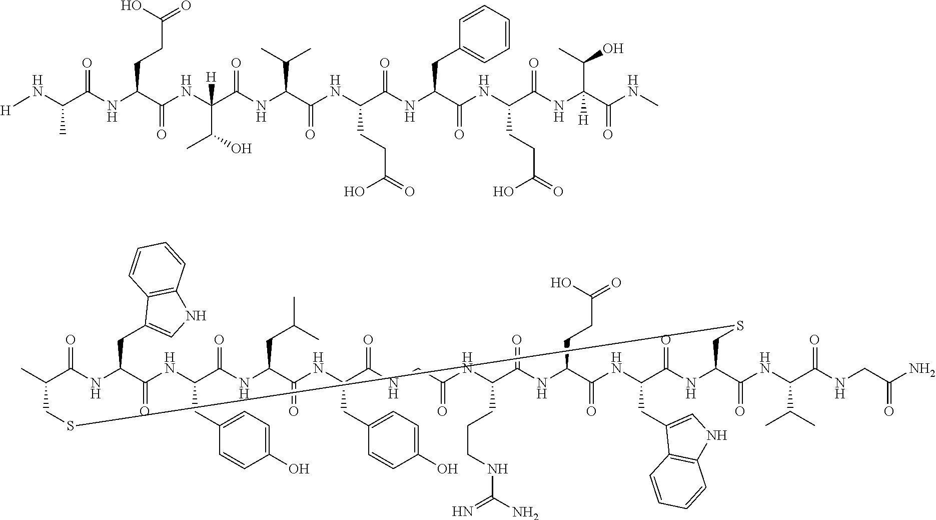 |
| PepSP1149 | AETVEFMRCYPWWYGPMCVR-CONH2 |
| | 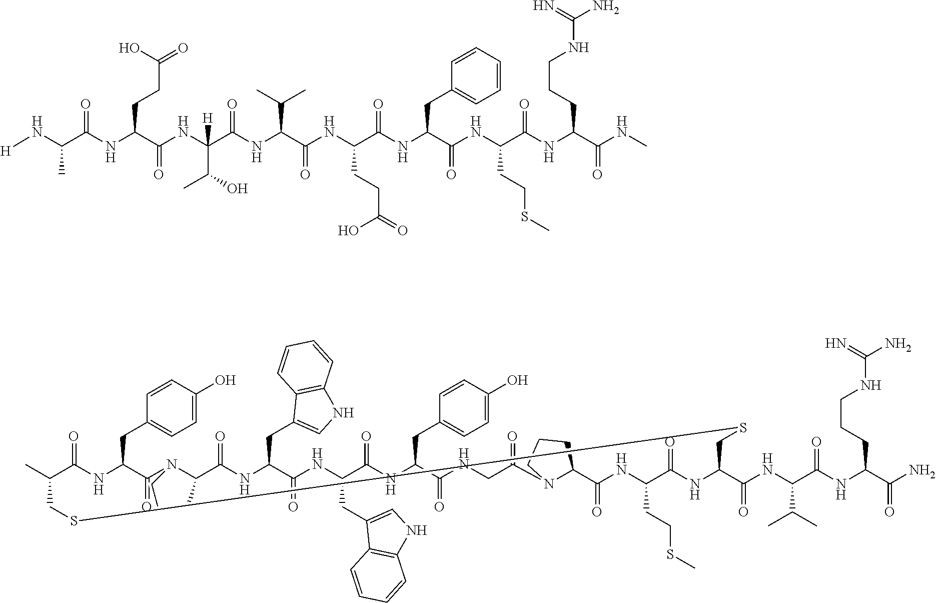 |
| PepSP1150 | AETVEFWGCLGMGYDRTCWW-CONH2 |
| | 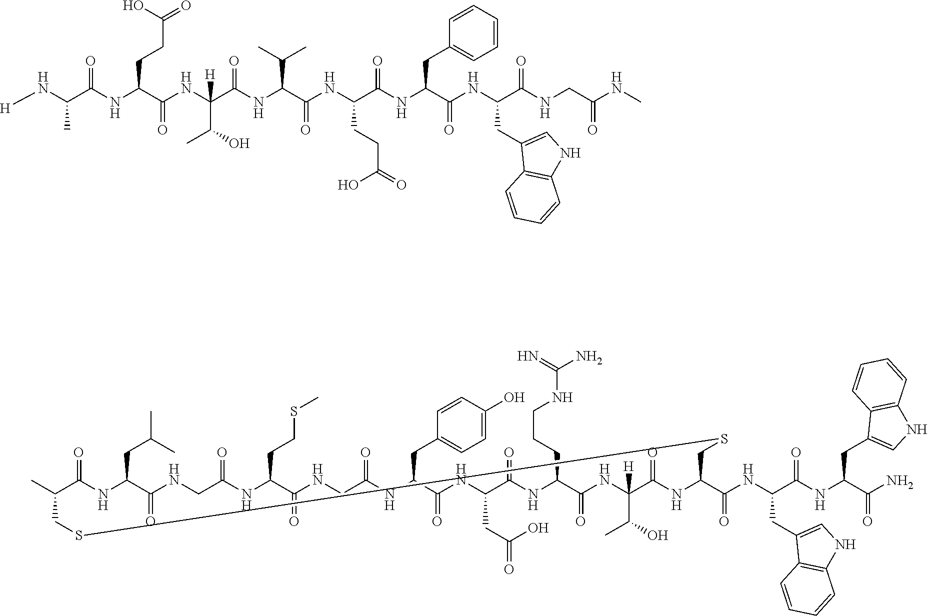 |
| PepSP1151 | AETVEFWTCYWTEGVSGCRW-CONH2 |
| | 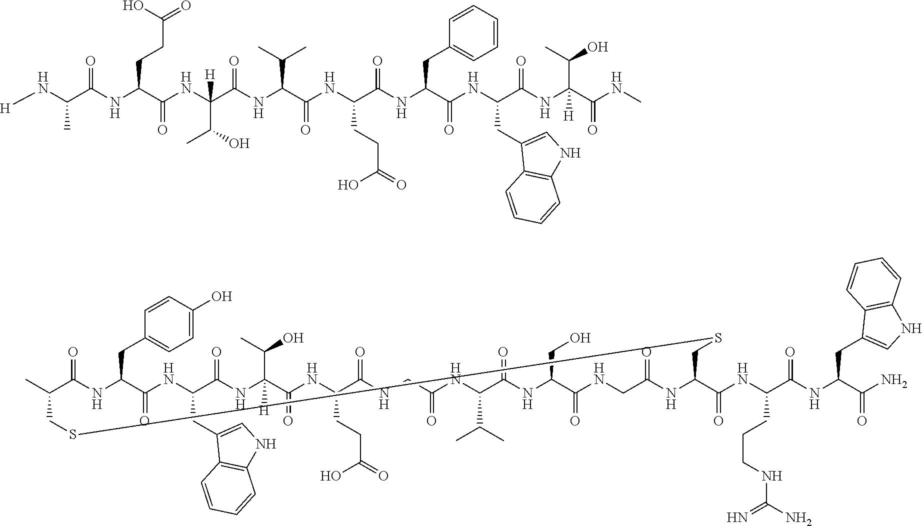 |
| PepSP1152 | AETVEFWTCWTVYGKSSCWD-CONH2 |
| | 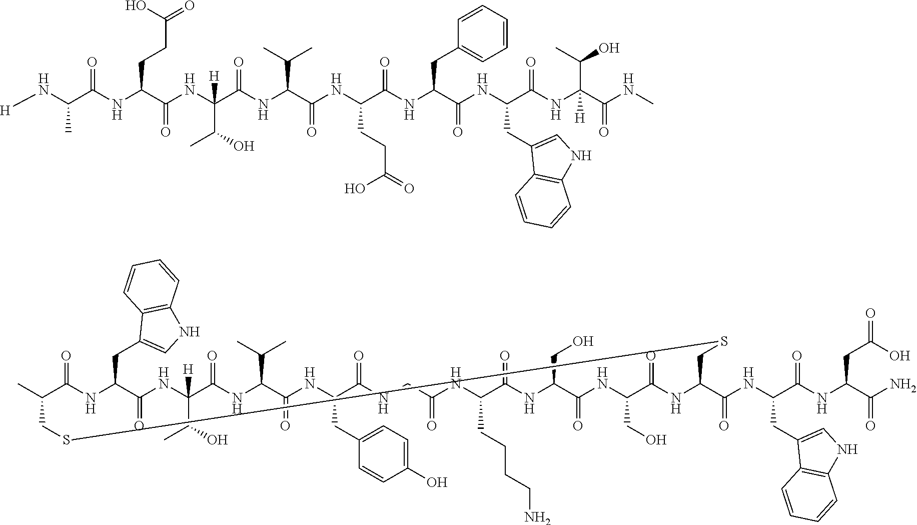 |
| PepSP1153 | AETVEFYGCWWDAYGSTCTW-CONH2 |
| | 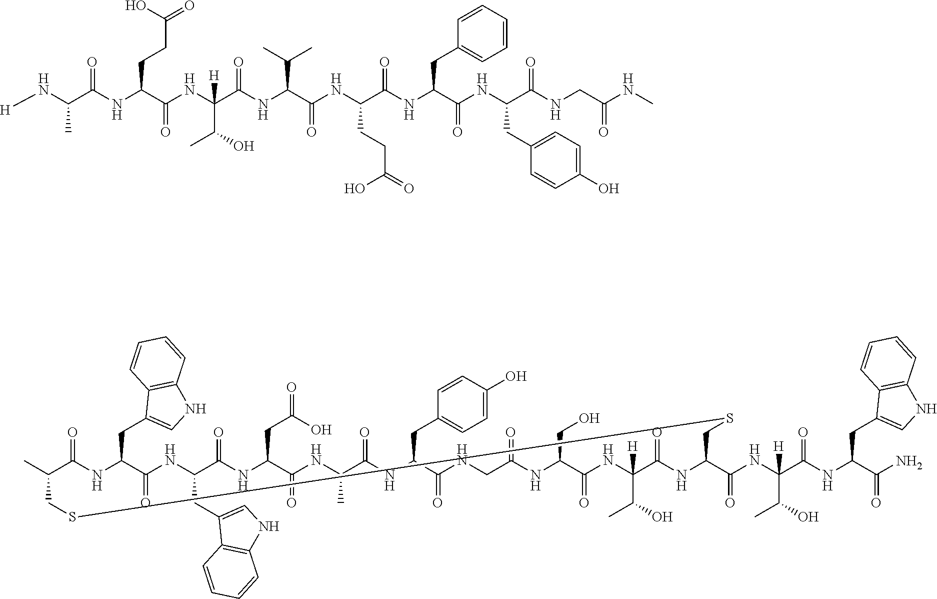 |
| PepSP1154 | AETVEFYGCDLWKGWTVCGY-CONH2 |
| | 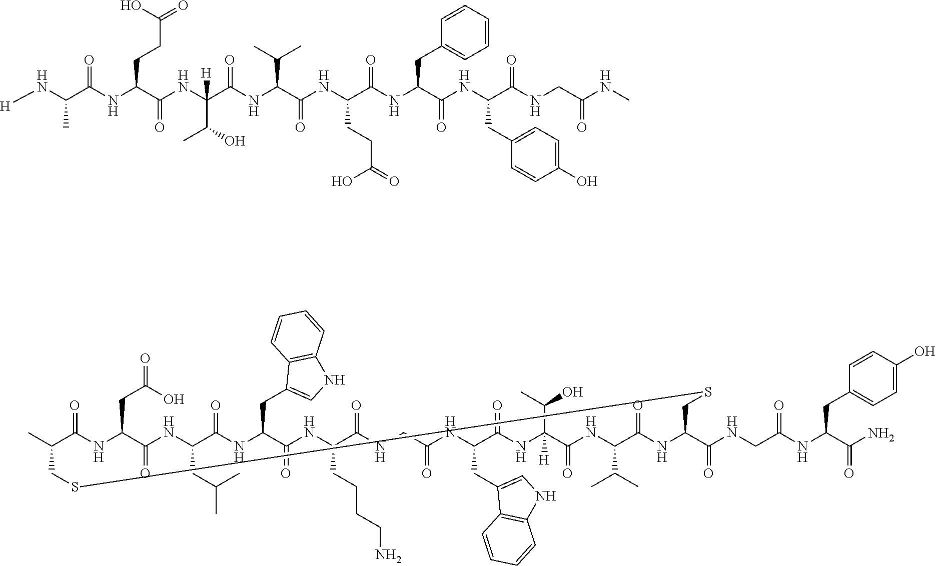 |
| PepSP1155 | AETVEYWRCDWYEETVACGT-CONH2 |
| | 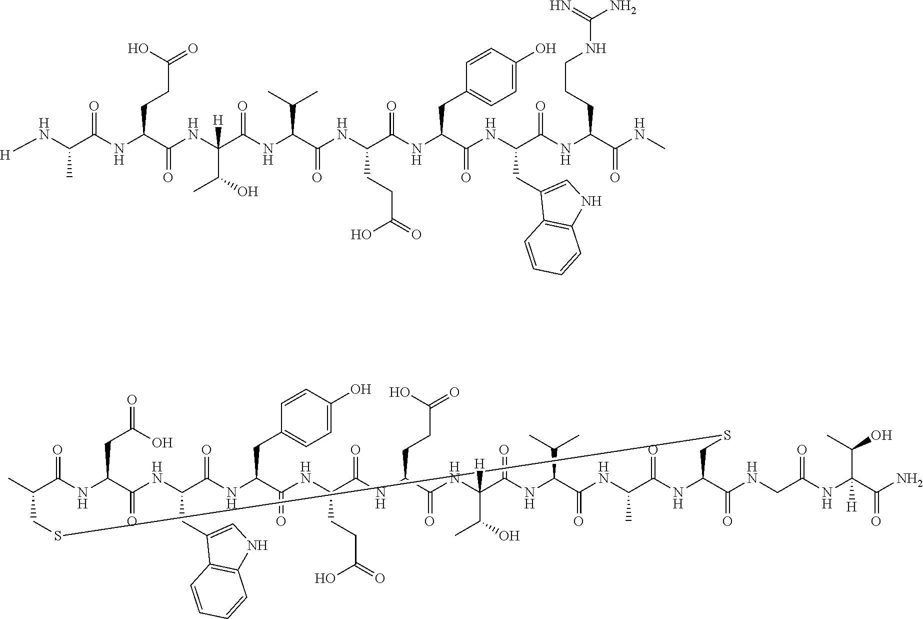 |
| PepSP1156 | AETVEFRYCTHTQTYVQCQD-CONH2 |
| | 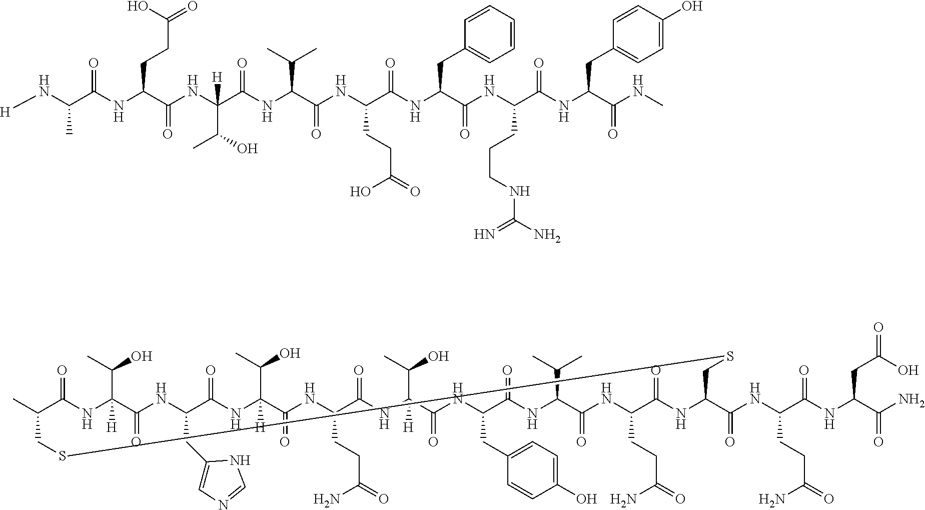 [SMILES: CNC(=O)[C@H](Cc1ccc(O)cc1)NC(=O)[C@H](CCCNC(=N)N)NC(=O)[C@H](Cc1ccccc1)NC(=O)[C@H](CCC(=O)O)NC(=O)[C@@H](NC(=O)[C@@H](NC(=O)[C@H](CCC(=O)O)NC(=O)[C@H](C)N)[C@@H](C)O)C(C)C ; CC(C)[C@@H]1NC(=O)[C@H](Cc2ccc(O)cc2)NC(=O)[C@H]([C@@H](C)O)NC(=O)[C@H](CCC(N)=O)NC(=O)[C@H]([C@@H](C)O)NC(=O)[C@H](Cc2cnc[nH]2)NC(=O)[C@H]([C@@H](C)O)NC(=O)[C@@H](C)CSSC[C@@H](C(=O)N[C@@H](CCC(N)=O)C(=O)N[C@@H](CC(=O)O)C(N)=O)NC(=O)[C@H](CCC(N)=O)NC1=O] |
| PepSP1157 | AETVEFWSCHYSYWTVSCGT-CONH2 |
| | 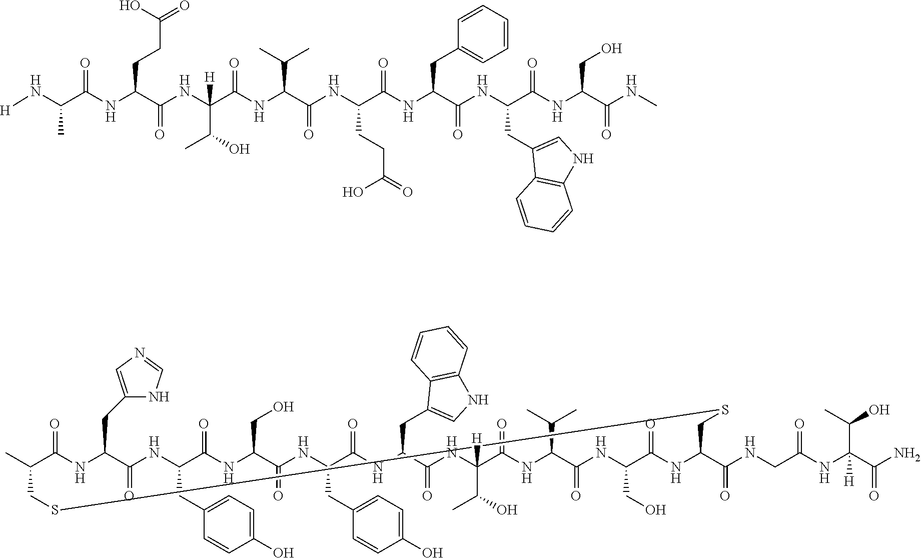 |
| PepSP1158 | AETVEFYGCSMDWRGVTCAW-CONH2 |
| | 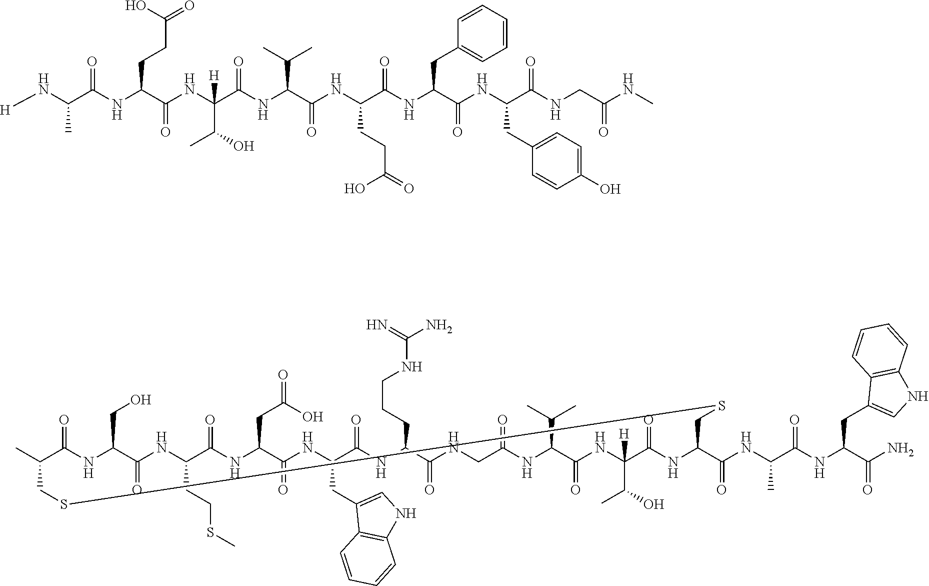 |
| PepSP1159 | AETVEFDYCLWAPDAWGCQN-CONH2 |
| | 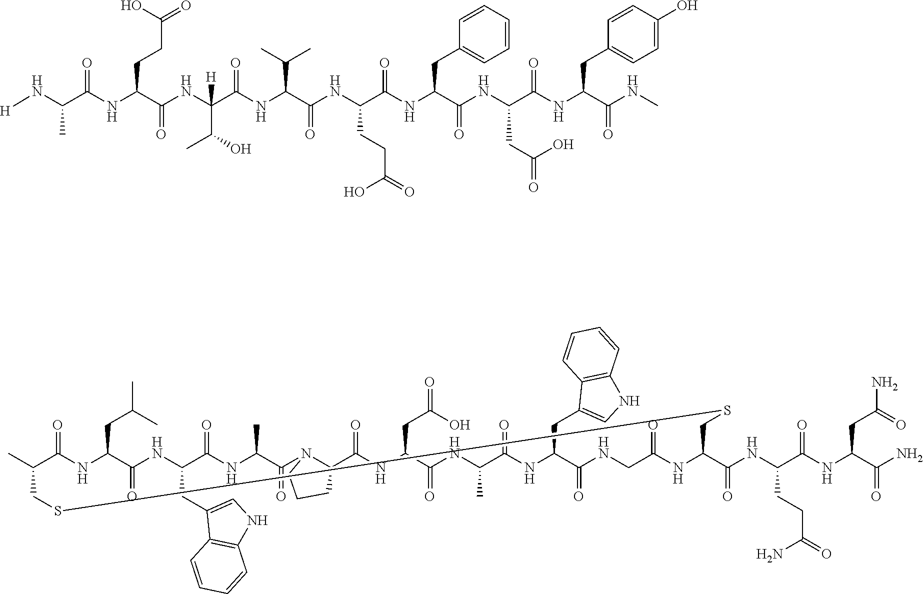 [SMILES: CNC(=O)[C@H](Cc1ccc(O)cc1)NC(=O)[C@H](CC(=O)O)NC(=O)[C@H](Cc1ccccc1)NC(=O)[C@H](CCC(=O)O)NC(=O)[C@@H](NC(=O)[C@@H](NC(=O)[C@H](CCC(=O)O)NC(=O)[C@H](C)N)[C@@H](C)O)C(C)C ; CC(C)C[C@@H]1NC(=O)[C@@H](C)CSSC[C@@H](C(=O)N[C@@H](CCC(N)=O)C(=O)N[C@@H](CC(N)=O)C(N)=O)NC(=O)CNC(=O)[C@H](Cc2c[nH]c3ccccc23)NC(=O)[C@H](C)NC(=O)[C@H](CC(=O)O)NC(=O)[C@@H]2CCCN2C(=O)[C@H](C)NC(=O)[C@H](Cc2c[nH]c3ccccc23)NC1=O] |
| PepSP1160 | AETVEFRGCTGNVYDWTCVW-CONH2 |
| | 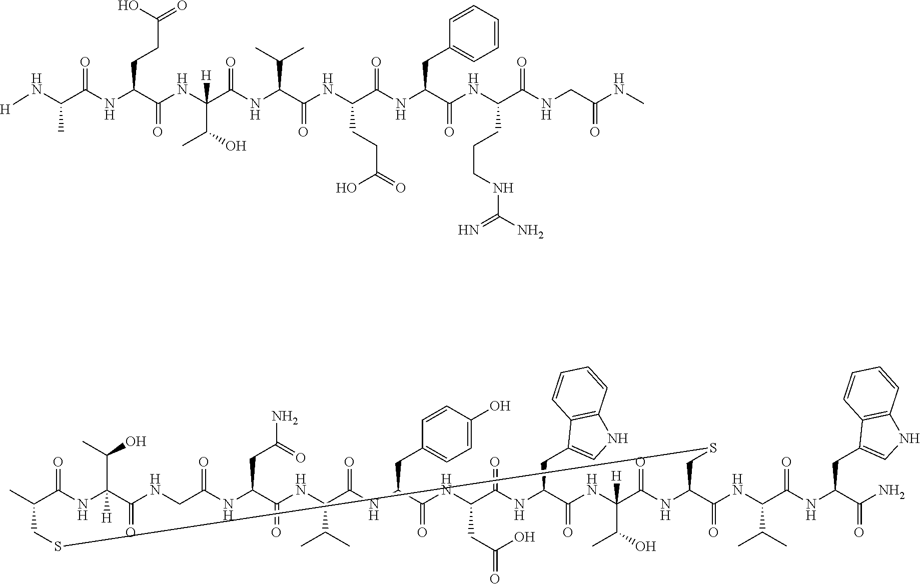 |
| PepSP1161 | AETVEFWDCWGNTDDWTCTW-CONH2 |
| | 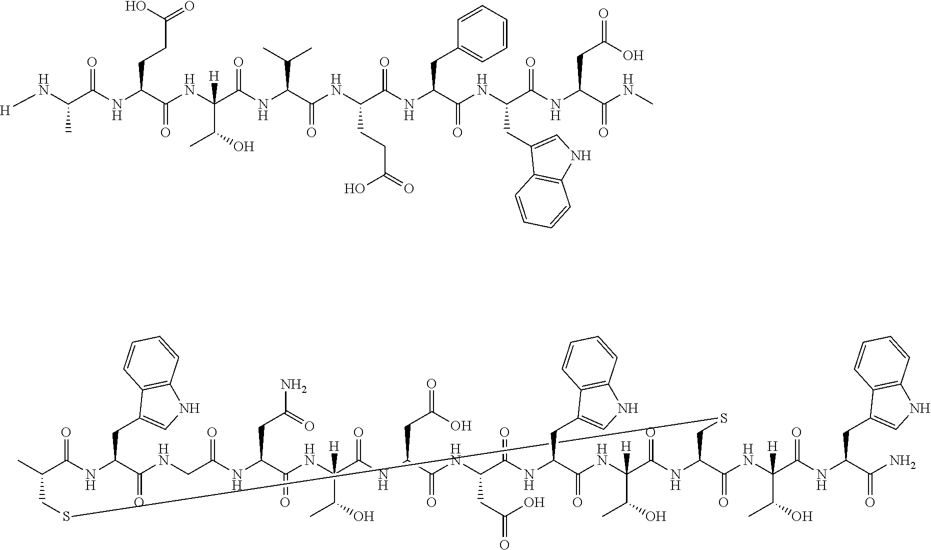 |
| PepSP1162 | AETVEFWTCHGNADEWTCSW-CONH2 |
| | 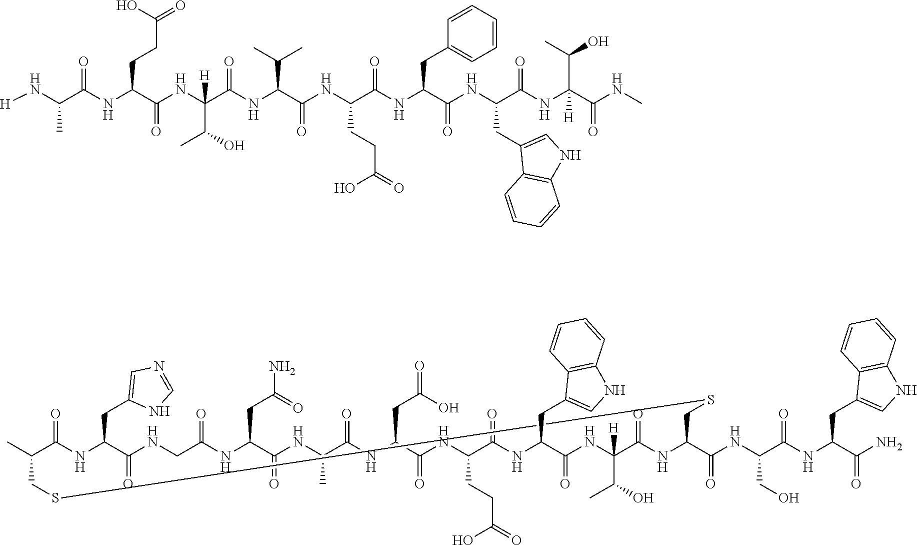 |
| PepSP1163 | AETVEFLGCHGDNSEWTCVW-CONH2 |
| | 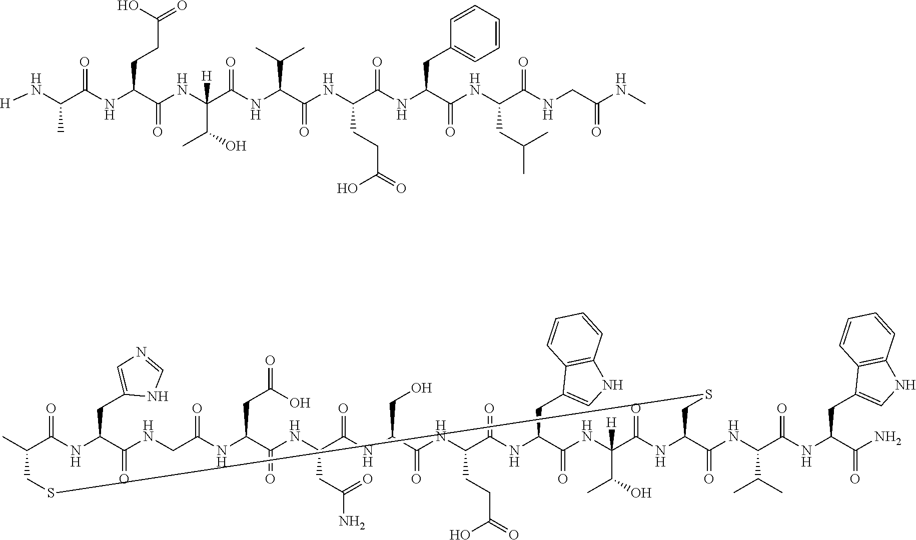 |
| PepSP1164 | AETVEFDGCKGDWYEWTCWW-CONH2 |
| | 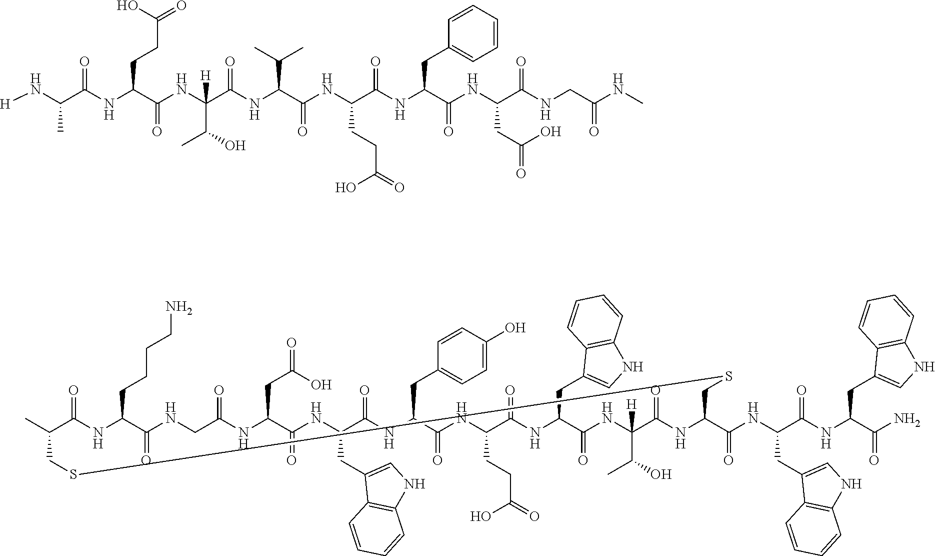 |
| PepSP1165 | AETVEFYGCRGGDGEWTCVW-CONH2 |
| | 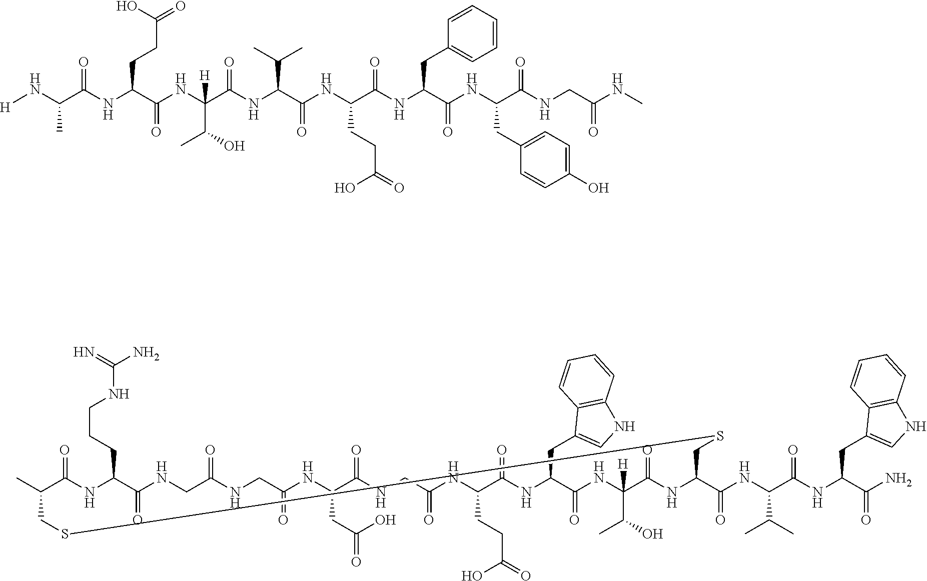 |
| PepSP1166 | AETVEFDYCYGDVEEWTCVY-CONH2 |
| | 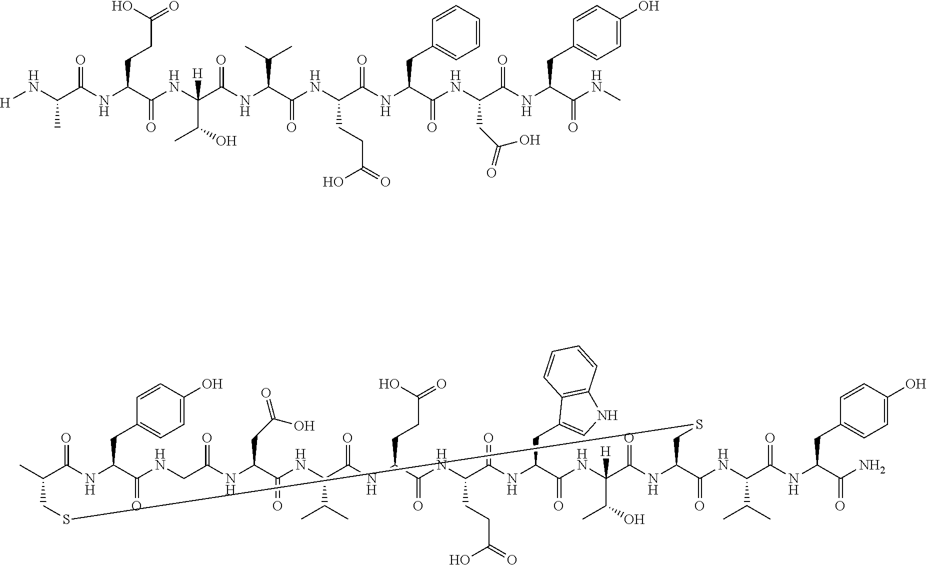 [SMILES: CNC(=O)[C@H](Cc1ccc(O)cc1)NC(=O)[C@H](CC(=O)O)NC(=O)[C@H](Cc1ccccc1)NC(=O)[C@H](CCC(=O)O)NC(=O)[C@@H](NC(=O)[C@@H](NC(=O)[C@H](CCC(=O)O)NC(=O)[C@H](C)N)[C@@H](C)O)C(C)C ; CC(C)[C@H](NC(=O)[C@@H]1CSSC[C@H](C)C(=O)N[C@@H](Cc2ccc(O)cc2)C(=O)NCC(=O)N[C@@H](CC(=O)O)C(=O)N[C@@H](C(C)C)C(=O)N[C@@H](CCC(=O)O)C(=O)N[C@@H](CCC(=O)O)C(=O)N[C@@H](Cc2c[nH]c3ccccc23)C(=O)N[C@@H]([C@@H](C)O)C(=O)N1)C(=O)N[C@@H](Cc1ccc(O)cc1)C(N)=O] |
| PepSP1167 | AETVEFYGCETYSGGWTCLW-CONH2 |
| | 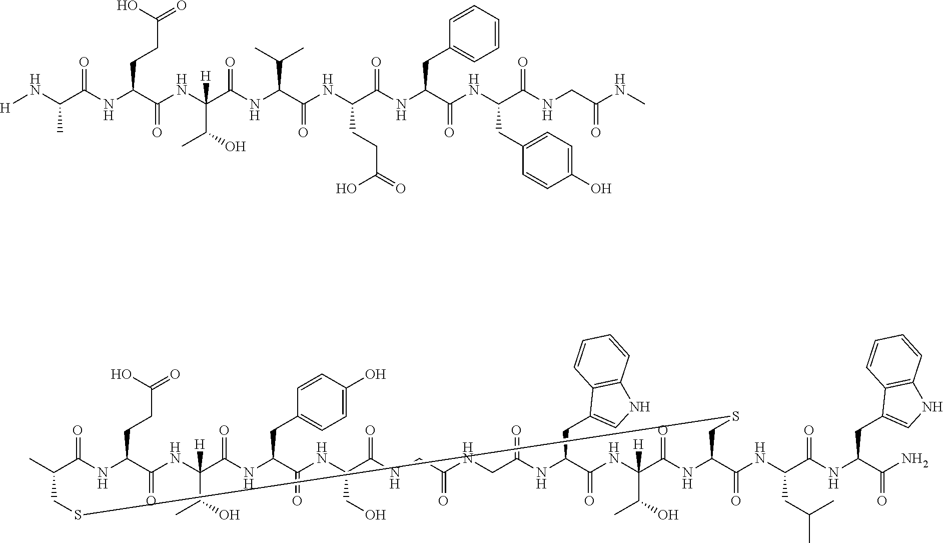 |
| PepSP1168 | AETVEFYGCKWDWKGWTCAW-CONH2 |
| | 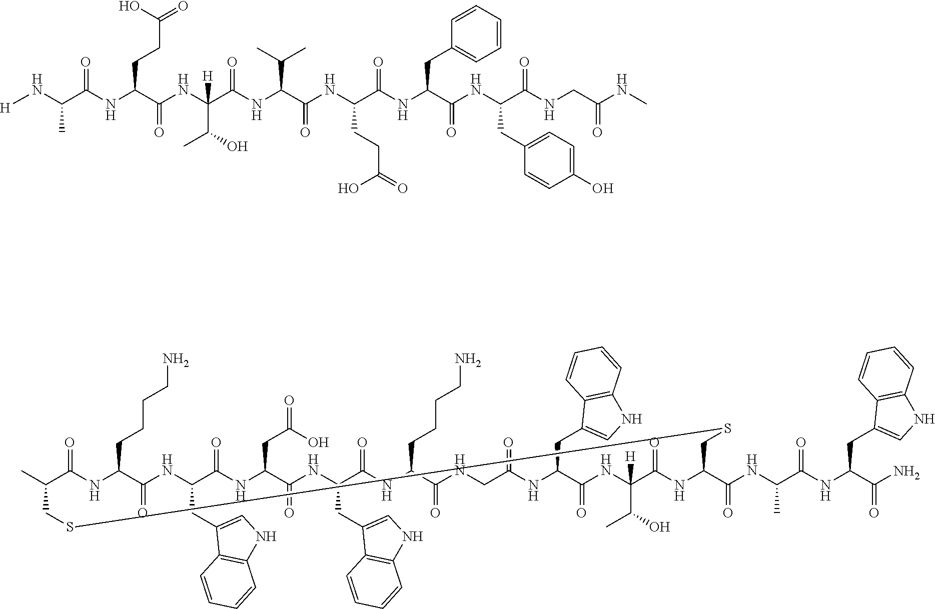 |
| PepSP1169 | AETVEFWGCNLDWYGWTCWW-CONH2 |
| | 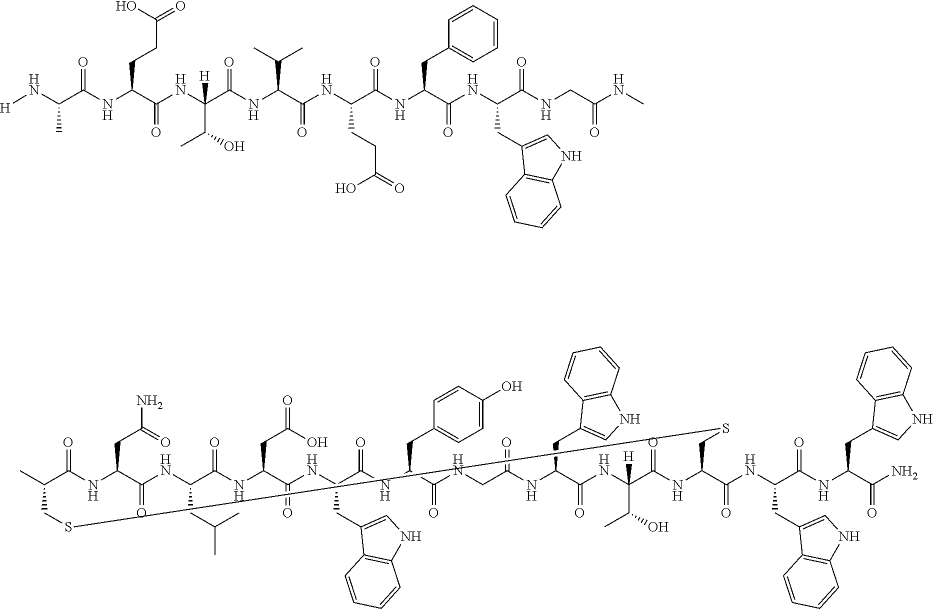 |
| PepSP1170 | AETVEFYGCSVNADGWTCSW-CONH2 |
| | 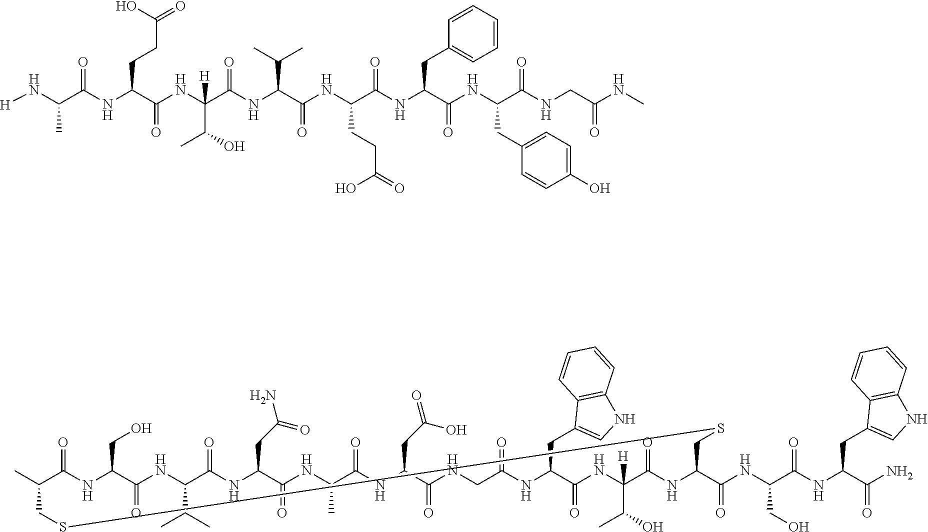 |
| PepSP1171 | AETVEFWWCNGNSENWTCTW-CONH2 |
| | 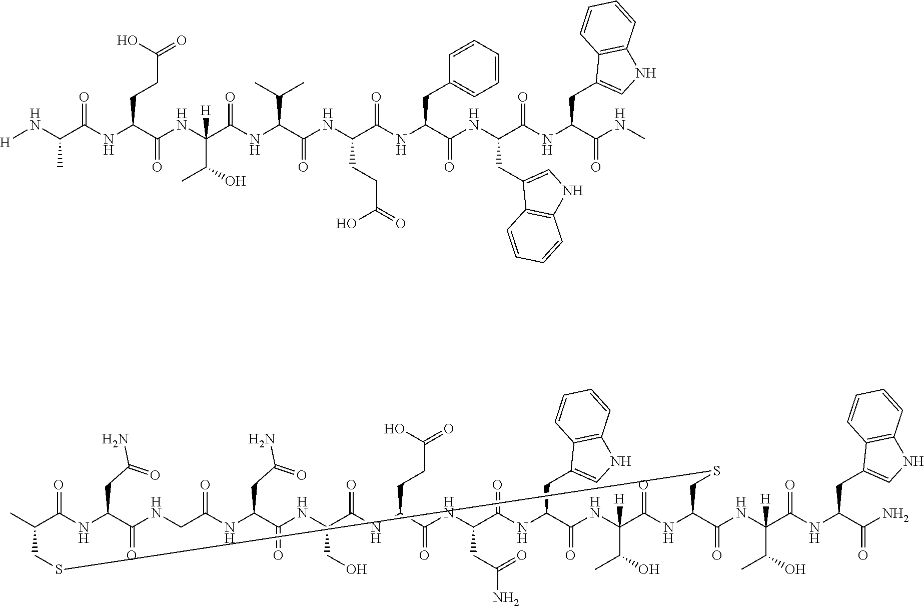 |
| PepSP1172 | AETVEFWSCVVYGDNWTCSS-CONH2 |
| | 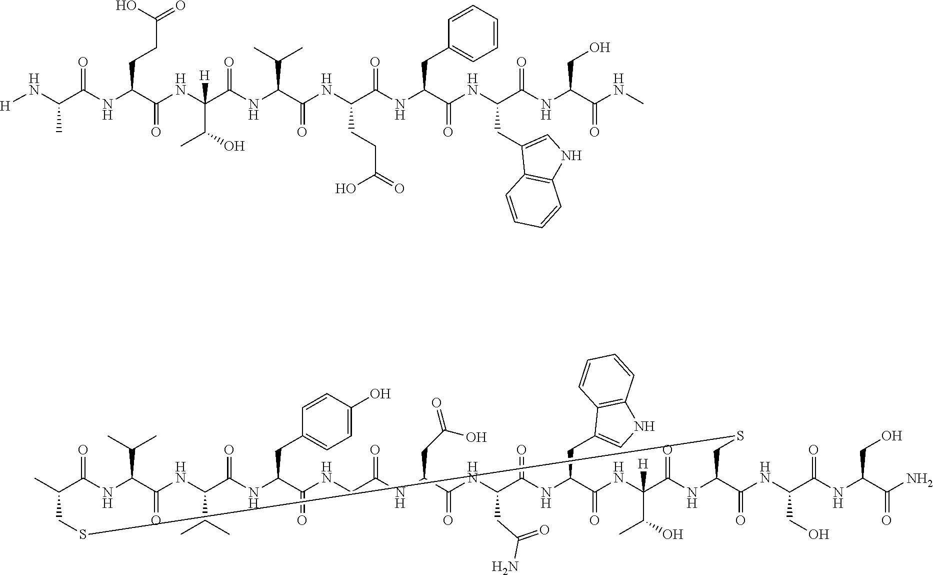 |
| PepSP1173 | AETVEFYGCYGEQEQWTCLW-CONH2 |
| | 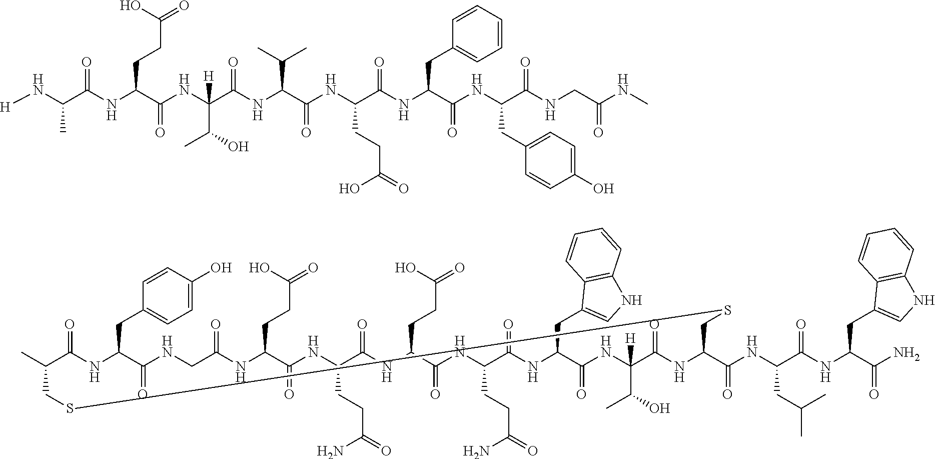 |
| PepSP1174 | AETVEFWSCQLGSGWWTCAW-CONH2 |
| | 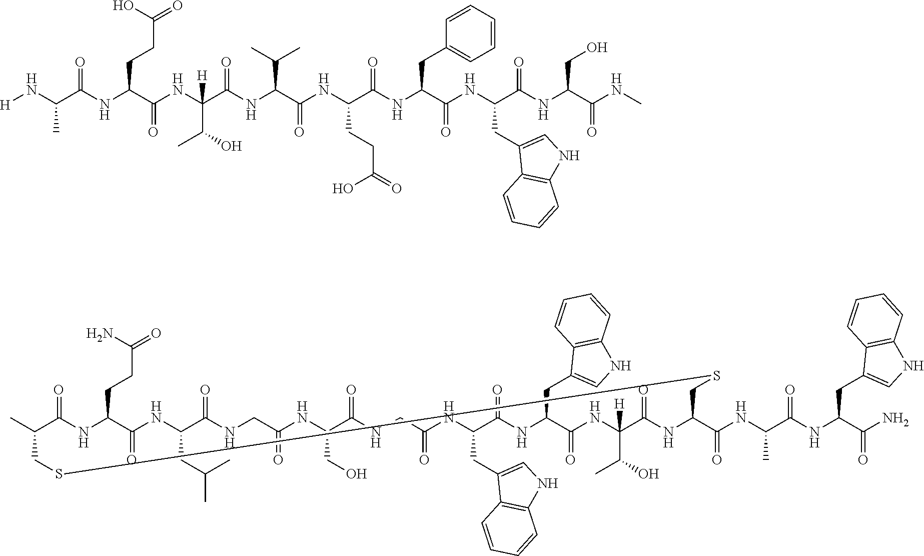 |
| PepSP1175 | AETVEFWTCWETTEGYGCNW-CONH2 |
| | 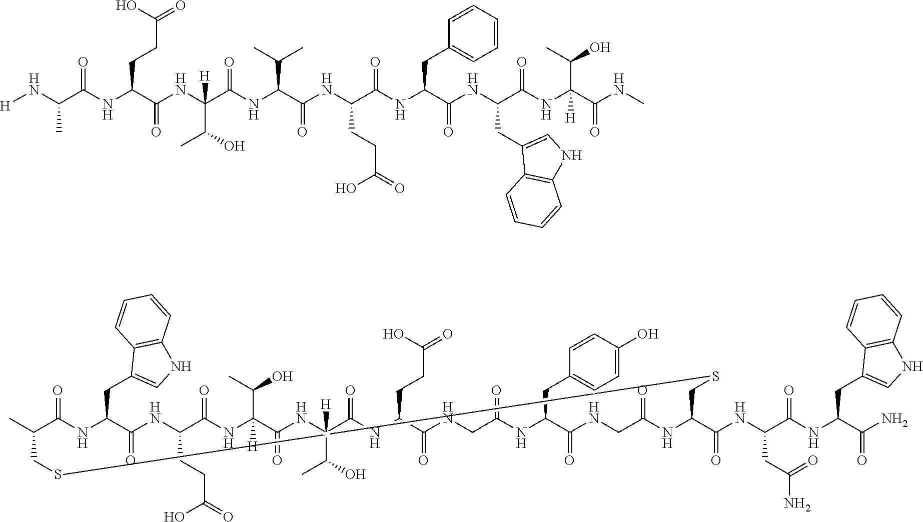 |
| PepSP1176 | AETVEFYGCQYYGWDYTCAW-CONH2 |
| | 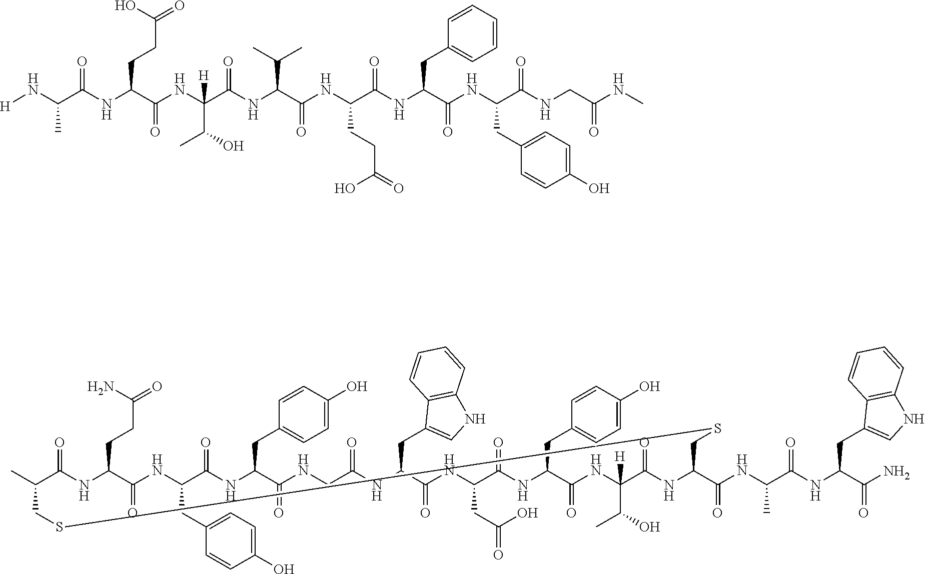 |
| PepSP1177 | AETVEFYGCWGNYNDYTCSW-CONH2 |
| | 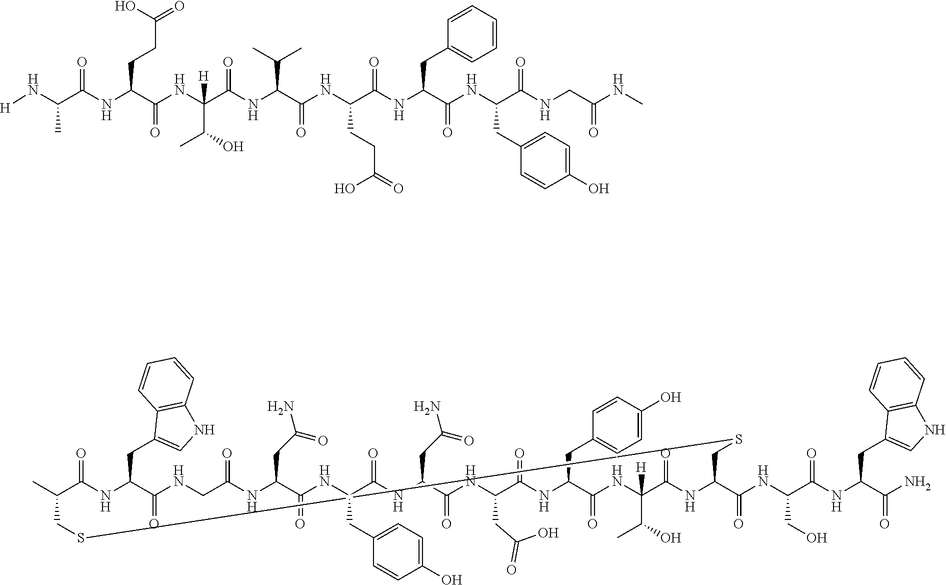 |
| PepSP1178 | AETVEFYGCYGDRNDYTCTW-CONH2 |
| | 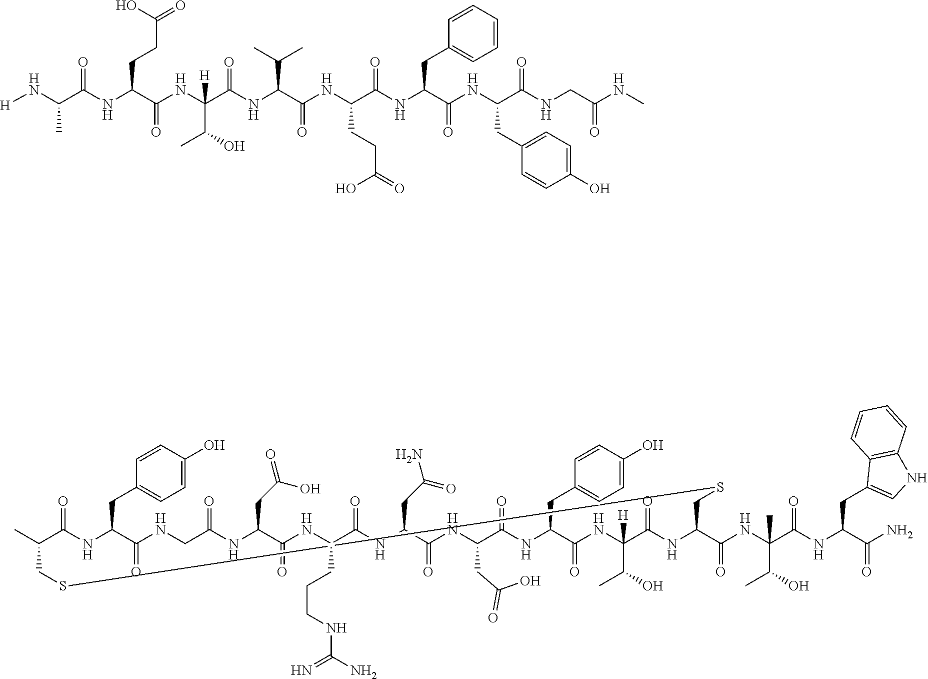 |
| PepSP1179 | AETVEFWGCTGGAWEYTCWW-CONH2 |
| | 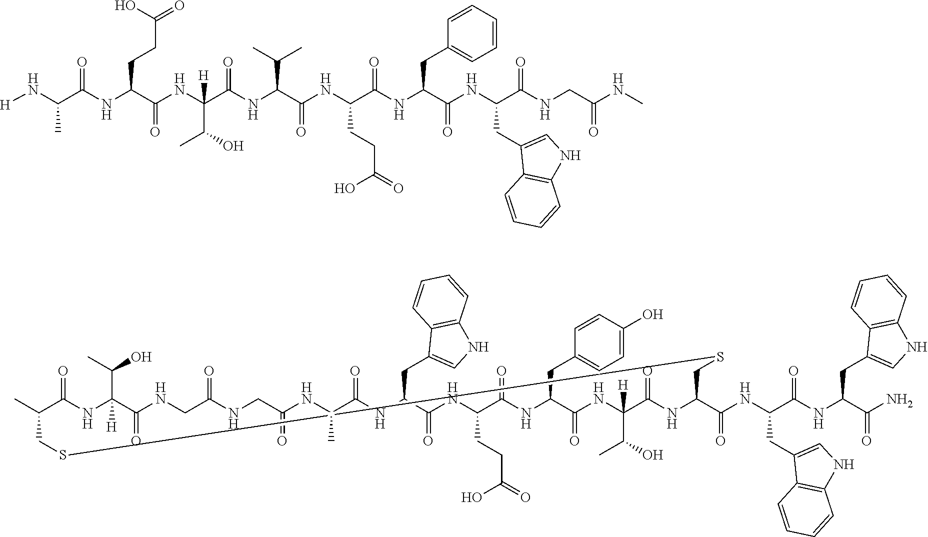 |
| PepSP1180 | AETVEFYGCSVTWGGYTCTW-CONH2 |
| | 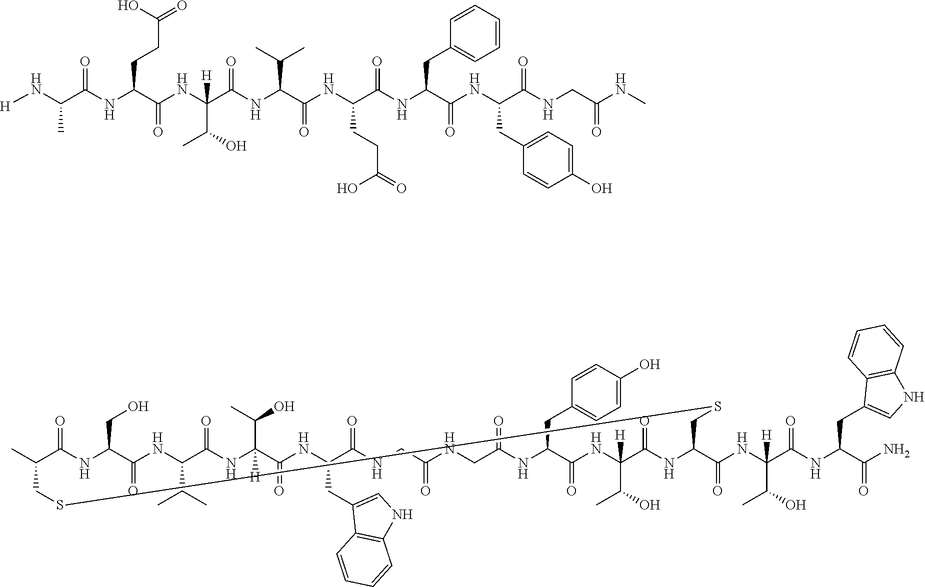 |
| PepSP1181 | AETVEFYNCWYKMGWYVCWS-CONH2 |
| | 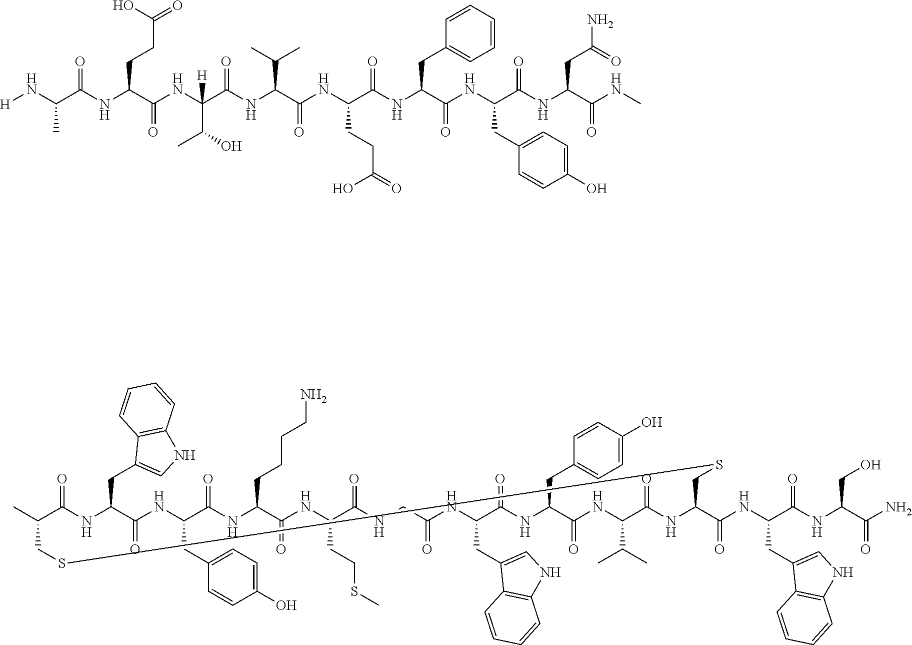 |
| PepSP1182 | AETVEFWSCKWWGGAADEYTYSCGW-CONH2 |
| | 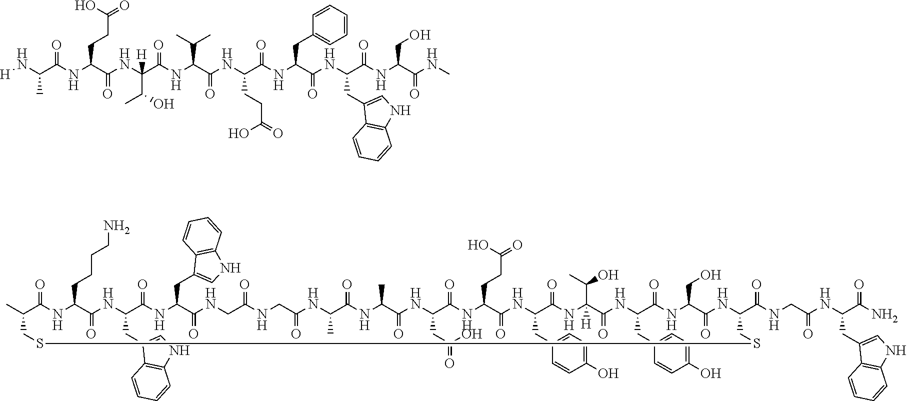 [SMILES: CNC(=O)[C@H](CO)NC(=O)[C@H](Cc1c[nH]c2ccccc12)NC(=O)[C@H](Cc1ccccc1)NC(=O)[C@H](CCC(=O)O)NC(=O)[C@@H](NC(=O)[C@@H](NC(=O)[C@H](CCC(=O)O)NC(=O)[C@H](C)N)[C@@H](C)O)C(C)C ; C[C@@H]1NC(=O)CNC(=O)CNC(=O)[C@H](Cc2c[nH]c3ccccc23)NC(=O)[C@H](Cc2c[nH]c3ccccc23)NC(=O)[C@H](CCCCN)NC(=O)[C@@H](C)CSSC[C@@H](C(=O)NCC(=O)N[C@@H](Cc2c[nH]c3ccccc23)C(N)=O)NC(=O)[C@H](CO)NC(=O)[C@H](Cc2ccc(O)cc2)NC(=O)[C@H]([C@@H](C)O)NC(=O)[C@H](Cc2ccc(O)cc2)NC(=O)[C@H](CCC(=O)O)NC(=O)[C@H](CC(=O)O)NC(=O)[C@H](C)NC1=O] |
| PepSP1183 | AETVEFCRAYSNWFYIC-NH2 |
| | 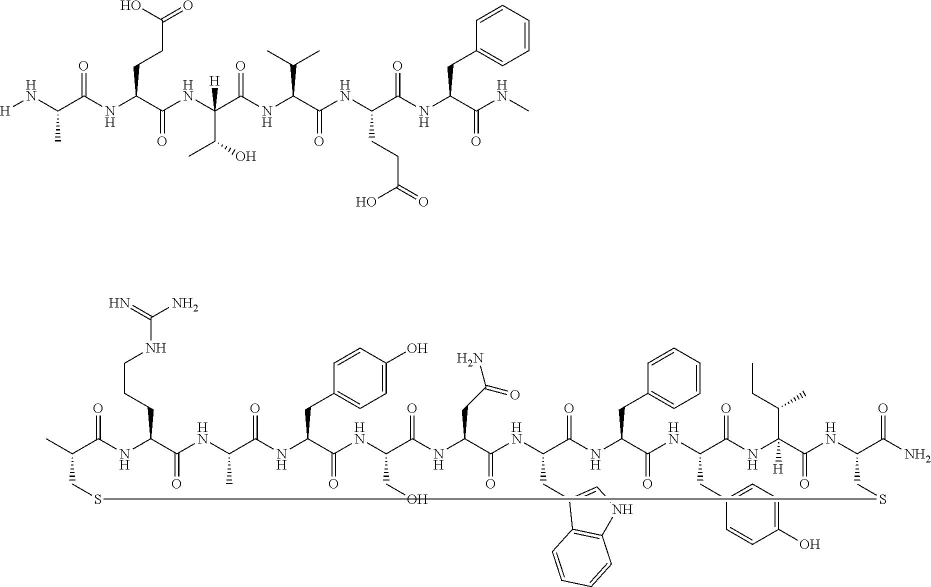 [SMILES: CNC(=O)[C@H](Cc1ccccc1)NC(=O)[C@H](CCC(=O)O)NC(=O)[C@@H](NC(=O)[C@@H](NC(=O)[C@H](CCC(=O)O)NC(=O)[C@H](C)N)[C@@H](C)O)C(C)C ; CC[C@H](C)[C@@H]1NC(=O)[C@H](Cc2ccc(O)cc2)NC(=O)[C@H](Cc2ccccc2)NC(=O)[C@H](Cc2c[nH]c3ccccc23)NC(=O)[C@H](CC(N)=O)NC(=O)[C@H](CO)NC(=O)[C@H](Cc2ccc(O)cc2)NC(=O)[C@H](C)NC(=O)[C@H](CCCNC(=N)N)NC(=O)[C@@H](C)CSSC[C@@H](C(N)=O)NC1=O] |
| PepSP1213 | WGCTGTWGNETCWW-CONH2 |
| | 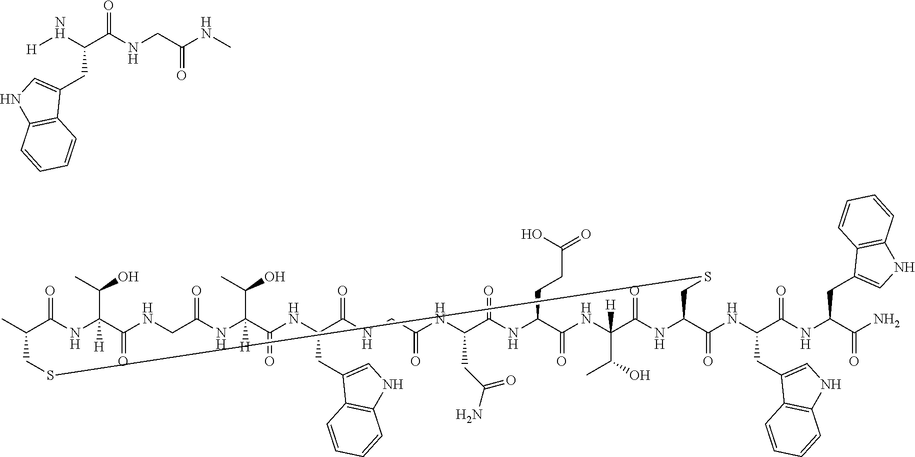 [SMILES: CNC(=O)CNC(=O)[C@@H](N)Cc1c[nH]c2ccccc12 ; C[C@H]1CSSC[C@@H](C(=O)N[C@@H](Cc2c[nH]c3ccccc23)C(=O)N[C@@H](Cc2c[nH]c3ccccc23)C(N)=O)NC(=O)[C@H]([C@@H](C)O)NC(=O)[C@H](CCC(=O)O)NC(=O)[C@H](CC(N)=O)NC(=O)CNC(=O)[C@H](Cc2c[nH]c3ccccc23)NC(=O)[C@H]([C@@H](C)O)NC(=O)CNC(=O)[C@H]([C@@H](C)O)NC1=O] |
| PepSP1214 | Ac-AETVEFWGCTGTWGNETCWW-CONH2 |
| | 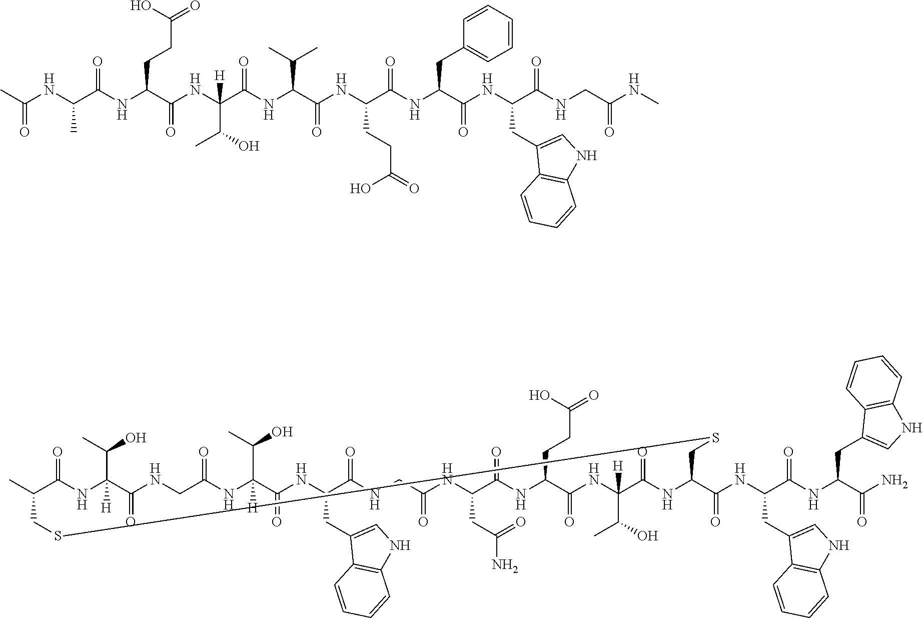 |
| PepSP1269 | WDCWGNTDDWTCTW-CONH2 |
| | 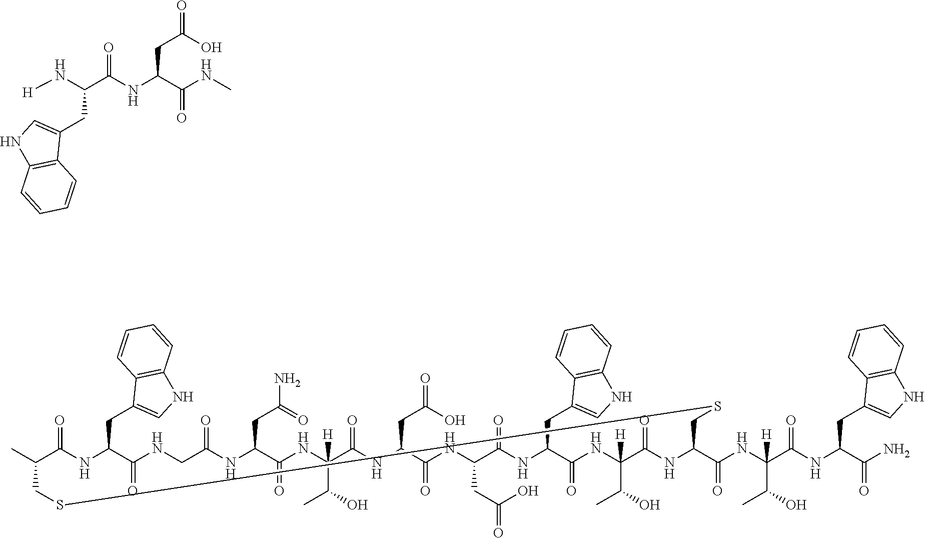 [SMILES: CNC(=O)[C@H](CC(=O)O)NC(=O)[C@@H](N)Cc1c[nH]c2ccccc12 ; C[C@H]1CSSC[C@@H](C(=O)N[C@H](C(=O)N[C@@H](Cc2c[nH]c3ccccc23)C(N)=O)[C@@H](C)O)NC(=O)[C@H]([C@@H](C)O)NC(=O)[C@H](Cc2c[nH]c3ccccc23)NC(=O)[C@H](CC(=O)O)NC(=O)[C@H](CC(=O)O)NC(=O)[C@H]([C@@H](C)O)NC(=O)[C@H](CC(N)=O)NC(=O)CNC(=O)[C@H](Cc2c[nH]c3ccccc23)NC1=O] |

*all peptides are cyclic with a disulfide bond between the two Cys residues

### Binding Kinetic Assay Using Biolayer Interferometry (BLI) and Surface Plasmon Resonance (SPR)

Binding kinetic assays were performed using Bio-layer interferometry (BLI) technology. The recombinant DLL3 ectodomain (rhDLL3-Fc) was biotinylated using EZ-Link Sulfo-NHS-LC-LC-Biotin Kit according to the manufacturer's protocol (Pierce by Thermo Fisher Scientific). The recombinant protein was incubated with an excess of NHS-LC-LC Biotin ester, in a molar ratio of 1:3 (protein: biotin), at 25° C. for 90 min. The un-reacted biotin ester was removed by subsequent buffer exchange on 0.5 ml Zeba spin desalting columns 7K MWCO (Pierce by Thermo Fisher Scientific).

Biomolecular interaction analysis was performed using an Octet Red 96e instrument (Forte Bio). Biotinylated hDLL3 was diluted at 12.5 μg/ml in 1× Kinetic Buffer (Forte Bio), then captured for 20 min on Streptavidin Dip and Read Biosensors (ForteBio) up to 8-10 nm, followed by sensor surface blocking with 10 μg/ml of biocytin for 60 s (Invitrogen by Thermo Fisher Scientific). To perform the assay, the peptides were diluted to final concentrations of 20, 4 and 0.8 μM in binding assay buffer (lx Kinetic buffer supplemented with 5% DMSO), and binding to DLL3 was assessed using 90 s association followed by 120 s dissociation. Peptides that showed positive binding were selected and tested in a dynamic range (10× dissociation constant (KD) to 0.1×KD) and dissociation was increased up to 300 s, to better evaluate affinity and binding kinetic parameters (e.g., kon, koff and KD). Sensorgrams were analyzed by Data Analysis HT 11.1 software (ForteBio): specific binding was obtained by subtracting binding from negative controls. Kinetic parameters were measured using a global fitting according 1:1 Langmuir binding isotherm equation.

The off-rate screening of the peptides was done using a Biacore T200 instrument (Cytiva, Sweden). Biotinylated rhDLL3-Fc was diluted at 12.5 μg/ml in HBS-P+1× buffer (Cytiva, Sweden) then captured on series S SA sensorchip (Cytiva, Sweden) up to 6000 RUs. Peptides were diluted at 300 nM in PBS-P+1× buffer (Cytiva, Sweden) supplemented with 1% DMSO and 0.1% BSA, then injected over the ligand for five minutes at 30 μl/min, finally dissociation allowed for 1 h. Sensorgrams were analyzed by Biacore T200 Evaluation Software 3.0 (Cytiva, Sweden): specific binding was obtained by subtracting unspecific binding to naked biosensors and a zero analyte concentration made of binding assay buffer (double referencing). Kinetic parameters of k_{off} were measured using a global fitting according to 1:1 Langmuir binding isotherm equation.

### Flow Cytometry

HEK293-huDLL3, CHO-huDLL3 and the corresponding parental cell lines were detached with EDTA 2.5 mM and re-suspended at 1.8×10⁶ cells/ml in FACS buffer (1×PBS, 2% FBS), distributed at 500 μl per testing tube and pelleted 5 min at 1300 rpm in a Heraeus Multifuge X3R centrifuge. For measuring binding of fluorescence labelled peptides to cell-surface hDLL3, cells were re-suspended in 500 μl FACS buffer in the presence of 100 nM AF647-labelled peptide (e.g., PepSP1215 or PepSP1216). In the competition experiments, cells were resuspended in 500 μl FACS buffer containing 30 nM peptide (e.g., PepSP1215) and increasing concentrations of an unlabelled peptide (e.g., PepSP1146, PepSP1270, PepSP1271 or PepSP1272) from 500 M to 4 nM were added. After 1 hour incubation at room temperature, cells were washed once with FACS buffer, centrifuged and re-suspended in FACS buffer/1% formaldehyde. Samples were acquired on a FACS ARIA flow cytometer (Becton Dickinson) and data analysed by FACS Express software.

### Results

DLL3 binding characteristics: 35 polypeptides were tested. Among them, 23 peptides (PepSP1146, 1147, 1151, 1155, 1157, 1158, 1159, 1160, 1161, 1162, 1163, 1164, 1165, 1166, 1167, 1168, 1170, 1171, 1173, 1176, 1177, 1178, and 1182) showed a dose-response saturable binding, fitted well according to a 1:1 Langmuir binding model. The remaining 12 showed poor binding (3) or heterogeneous binding (9) modes. Generally, peptides that had a Kd in the micromolar range (e.g., greater than 1 M) were considered poor binders while peptides that had a Kd in the single to double digit nM were considered good binders. Peptides that did not fit well to the 1:1 Langmuir binding model were considered to have heterogeneous binding. The 23 peptides were tested again in a wider dose-response curve, ranging approximately from 10×KD to 0.1×KD, and kinetic parameters were determined using a global fitting procedure according to a 1:1 Langmuir model. Affinity values were obtained as koff vs kon ratio. The characterized peptides showed an overall affinity of below 1 μM, and seven peptides (PepSP1146, 1178, 1163, 1161, 1147, 1171 and 1182) showed an affinity in the double-digit nM range, with a residence time ranging from 60 to 180 seconds. These seven peptides were tested again in three independent experiments. The data of the 23 peptides and PepSP1213, 1214, and 1269 are summarized in Table 4 below.

TABLE 4
Binding characteristics

| PEP ID | KD (nM) | RT* (sec) |
|---|---|---|
| 1146 | 68.9 ± 27.6 | 184.5 ± 7.8 |
| 1161 | 42.5 ± 10 | 87.8 ± 6 |
| 1178 | 146.8 ± 46.7 | 75.9 ± 9 |
| 1163 | 68.7 ± 18.4 | 166.8 ± 19.5 |
| 1171 | 70 ± 19 | 110.1 ± 32.4 |
| 1182 | 75.8 ± 15.5 | 96.6 ± 3.8 |
| 1147 | 59.0 ± 3.4 | 65.6 ± 6.8 |
| 1151 | 125 | 167 |
| 1155 | 155.2 | 177 |
| 1157 | 123 | 89 |
| 1158 | 643 | 31 |
| 1159 | 900 | 6 |
| 1160 | 494.5 | 47 |
| 1161 | 43 | 87.8 |
| 1162 | 159 | 51 |
| 1163 | 69 | 166.8 |
| 1164 | 125 | 125 |
| 1165 | 263 | 66 |
| 1166 | 144.6 | 48 |
| 1167 | 399 | 47 |
| 1168 | 568 | 44 |
| 1170 | 165.6 | 43 |
| 1173 | 105.6 | 101 |
| 1176 | 207 | 67 |
| 1177 | 236.5 | 52 |
| 1213 | 7498 | 1.36 |
| 1214 | 34.5 | 233 |
| 1269 | 181.1 | 20 |

RT: residence time

The peptides were functionalized at the N- or C-terminal to evaluate their impact on binding to DLL3. Acetylation of the N-terminus is a desirable feature for the peptides as it blocks the N-terminal amino group reactivity, which can enable a more straightforward synthetic process for further modifying the peptides (e.g., conjugation to radiolabeled moieties). Surface Plasmon Resonance (SPR) showed that neither acetylation of the N-terminal sequence nor the C-terminal amidation or amination perturbed the binding properties of the peptides.

In addition, the N-terminal amino acids (AETVEF or AETVE) common to all unique sequences were deleted to evaluate the impact on binding. It was found that the amino acids can play a role for binding of some of the peptides to DLL3, however, the amino acids are not required for all of the peptides to bind to DLL3. See e.g., the binding data for PepSP1213 and PepSP1214, and the binding data for PepSP1161 and 1269.

### Example 3. Labeling, Dimerization of DLL3 Binding Peptides and Characterizations Thereof

### Materials and Methods

Various exemplary reagents (e.g., linkers, click chemistry reagents, fluorescence agents) that were used in this series of experiments are listed below. These reagents are commercially available.

Linkers/spacers: Trioxatridecan-succinamic acid (Ttds), Gly-Gly-Ttds, Gly-Gly-Ttds-K, Gly-Gly-Ttds-K(PEG4), Gly-Gly-Ttds-K(PEG3), Gly-Gly-Ttds-K(PEG4-PEG3), Gly-Gly-Ttds-K(Ttds-Ttds), Gly-Gly-Ttds-K(PEG4-DBCO), Gly-Gly-Ttds-K(Ttds-Ttds-PEG3), PEG4-DBCO, PEG linkers such as PEG3, PEG4, PEG6, bis-propargyl-PEG6, bis-propargyl-PEG14 and bis-propargyl-PEG18.
Click chemistry reagents: homopropargylglycine (hPra), Fmoc-HPra-OH, Fmoc-Lys(N3)-OH
Fluorescence agents: AFDye™ 647 Dibenzocyclooctyne (DBCO), Cy5 DBCO
PepSP1396 and PepSP1342: At the end of PepSP1324 assembly, Fmoc-N-amido-PEG4-acid (CAS 557756-85-1) was acylated on the resin with a 4-fold excess using equimolar amounts of DIC and HOAt in DMF as activators, followed by Fmoc deprotection (piperidine 20% in DMF, 3×3 min) to yield PepSP1396. Similarly, at the end of PepSP1324 assembly, Fmoc-Ttds-OH (CAS 172089-14-4) was acylated on the resin with a 4-fold excess using equimolar amounts of DIC and HOAt in DMF as activators followed by Fmoc deprotection (piperidine 20% in DMF, 3×3 min); the treatment is repeated for the second Fmoc-Ttds-OH present in the sequence to yield PepSP1342.
Fluorescence labeling: PepSP 1146 and 1171 were used for labeling and dimerization experiments. At the beginning of the synthesis, the non-natural amino acid Fmoc-Lys(N3)-OH (CAS 159610-89-6) was acylated on the resin followed by acylation of the linker Fmoc-Ttds-OH using standard methods and the peptides were synthesized using the same procedure described above. Next, the peptides were extended at the C-terminal with a linker (Ttds) and a Lys(N3) group to generate peptide precursors PepSP1273 and PepSP1274 (see Table 5 below). The peptide precursors were then labeled with a fluorescent agent, e.g., using copper free click chemistry with AlexaFluor647DBCO, to make PepSP1215 and PepSP1216.

Specifically, for labeling with AlexaFluor647, the purified peptide precursors (final peptide concentration 30 mg/ml) were conjugated to AFDye™ 647 DBCO (Click Chemistry Tools Cat.1302) by incubation with 1.3 eq of Alexa-DBCO (dissolved in DMSO). The reaction, left overnight, was monitored by UPLC analysis on a BEH300 C4 Acquity Waters column (2.1×100 mm, 1.7 μm) with a gradient of 20% B-20% B (1 min), 20% B-90% B (4 min), 90% B-90% B (0.2 min) and 25% B-25% B (1 min) 25% B-45% B (4 min), 45% B-95% B (0.3 min) (eluents: A=H2O+0.1% TFA; B=CH3CN+0.1% TFA); flow: 0.4 mL/min; A: 214 nm; temp: 45° C.; MS: Waters Acquity ESI+, single quandrupole. The reaction was then diluted with DMSO and loaded on a reversed-phase HPLC using a Delta Pak C4 200×25 mm 300 A 15 um column and the linear gradient: 15% B-15% B-30% B in 20 min (eluents: A=H2O+0.1% TFA; B=CH3CN+0.1% TFA; Flow: 50 mL/min; λ: 214 nm). The analytical characterization was performed on a BEH300 C4 Acquity Waters 2.1×100 mm, 1.7 μm column with a gradient of 25% B-25% B (1 min) 25% B-45% B (4 min), 45% B-80% B (0.2 min).

### Dimerization

It is believed that dimerization of the peptides can improve the binding property of the peptides due to, e.g., improved avidity. To this end, peptide dimers were synthesized and tested. Precursor PepSP1273 was homodimerized using 3 linkers, Bis-propargyl-PEG6, Bis-propargyl-PEG14, and Bis-propargyl-PEG18, as each linker has two terminal alkyne functionalities that react with the peptide precursor PepSP1273 by copper catalyzed click chemistry to make the homodimers PepSP1270, 1271, and 1272. Specifically, 1 part of the peptide azido precursor (PepSP1273) was incubated with 0.5 molar equivalent part of bispropargyl-PEGx (x is 6, 14 or 16), 3 molar equivalent parts of CuSO4, and 5 molar equivalent parts of eq Na Ascorbate. The reaction was carried out in DMSO with salts dissolved in water (water content<10%) with a final peptide concentration of 30 mg/ml. The reaction, complete after 5 min, was monitored by UPLC analysis on a BEH300 C4 Acquity Waters column (2.1×100 mm, 1.7 μm) with a gradient of 30% B-30% B (1 min), 30% B-50% B (4 min), 50% B-90% B (0.2 min) (eluents: A=H2O+0.1% TFA; B=CH3CN+0.1% TFA); flow: 0.4 mL/min; A: 214 n; temp: 45° C.; MS: Waters Acquity ESI+, single quandrupole. The reaction was diluted with DMSO and TFA and loaded on a preparative HPLC Waters system using the following conditions.

PepSP1270: gradient: 20% B-20% B (5 min) 20% B-40% B in 25 min; column: X Bridge C4 150×19 mm 300 A 5 um; eluents: A=H2O+0.1% TFA; B=CH3CN+0.1% TFA; flow: 20 mL/min; λ: 214 nm; PepSP1271: gradient: 25% B-25% B (5 min) 25% B-45% B in 25 min; column: Daisogel C4 200×20 mm 200 A 5 um; eluents: A=H2O+0.1% TFA; B=CH3CN+0.1% TFA; flow: 15 mL/min; λ: 214 n; PepSP1272: gradient: 20% B-20% B (5 min) 20% B-40% B in 25 min; column: Delta Pak C4 200×25 mm 300 A 15 um; eluents: A=H2O+0.1% TFA; B=CH3CN+0.1% TFA; flow: 50 mL/min; λ: 214 nm.

To improve dimerization and labeling reactions, it is desirable to incorporate in the same linker molecule: a) the best format and b) the ability to undergo straightforward conjugation reaction(s) with a radioactive moiety (e.g., 18F) for imaging or therapeutic purposes. To achieve this, a tri-functionalized linker was designed and synthesized using Scheme I shown below.

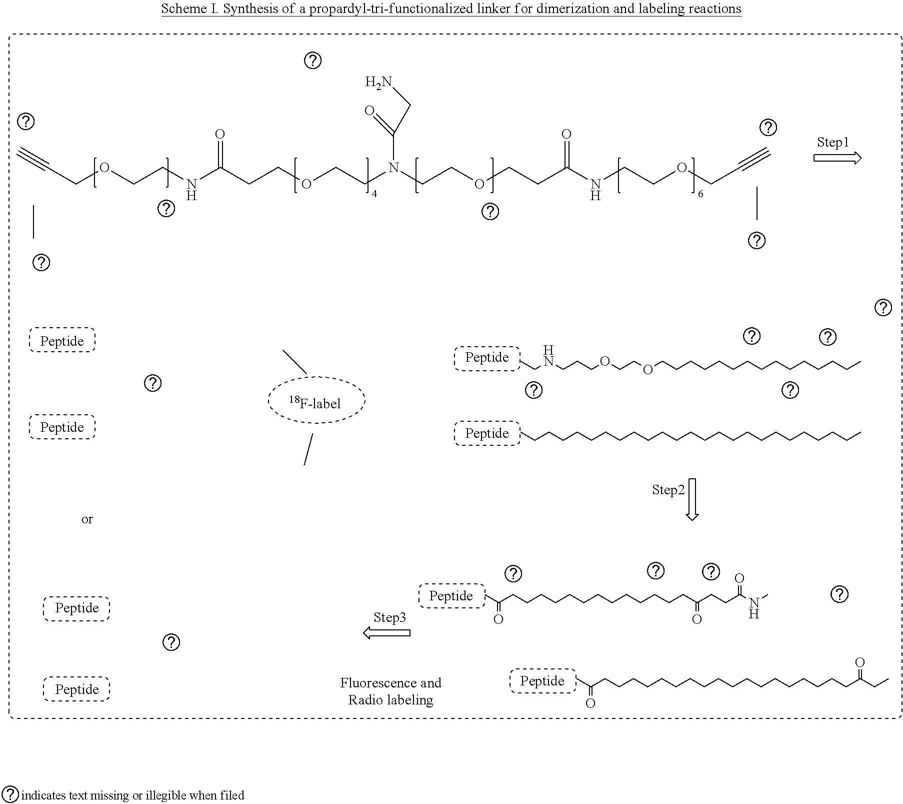

Starting from the linker PEG18, tri-functionalized linkers were synthesized according to Scheme I and functionalized with a DBCO or BCN moiety. The linkers having DBCO or BCN moiety were then used to conjugate a radioisotope (e.g., 18F) to the peptide using copper-free click chemistry. PepSP1384: The peptide azido precursor PepSP1274 (1 eq) was incubated with 0.5 eq of bis-propargyl-PEG18, 2 eq CuSO4, and 2 eq Na Ascorbate. The reaction was performed in DMSO with salts dissolved in water (water content<10%) with a final peptide concentration of 20 mg/ml. The reaction, complete after 5 min, was monitored by UPLC analysis on a BEH300 C4 Acquity Waters column (2.1×100 mm, 1.7 μm) with a gradient of 30% B-30% B (1 min), 30% B-50% B (4 min), 50% B-90% B (0.2 min) (eluents: A=H2O+0.1% TFA; B=CH3CN+0.1% TFA); flow: 0.4 mL/min; λ: 214 nm; temp: 45° C.; MS: Waters Acquity ESI+, single quadrupole. The reaction was diluted with DMSO and TFA and loaded on a reversed phase HPLC using a Delta Pak C4 200×25 mm 300 A 15 um column and the linear gradient: 25% B-40% B in 20 min (eluents: A=H2O+0.1% TFA; B=CH3CN+0.1% TFA; flow: 50 mL/min; λ: 214 nm). The analytical characterization was performed on a BEH300 C4 Acquity Waters 2.1×100 mm, 1.7 μm column with a gradient of 30% B-30% B (1 min), 30% B-50% B (4 min), 50% B-90% B (0.2 min); flow: 0.4 mL/min; λ: 214 nm; temp: 45° C.; MS: Waters Acquity ESI+, single quadrupole. PepSP1321 and PepSP1324 were synthesized similarly.

The PepSP1343, PepSP1344 and PepSP1371 dimers were prepared using PepSP1318 as a precursor. Specifically, PepSP1318 was dimerized with a suitable PEG linker, Propargyl-Tri-functionalized linker (see scheme I) to make PepSp1343. PepSP1344 and PepSP1371 were made from PepSP1343 and a suitable linker derivatized with either a DBCO or BCN group (PEG4-DBCO or PEG3-BCN).

Specifically, for PepSP1343, PepSP1318 (precursor) (leg) was incubated with 0.6 eq of propargyl-tri-functionalized linker (synthesized according to Scheme III described herein below) and 1.9 eq CuSO4 with 1.9 eq Na Ascorbate were added to the solution. The reaction was performed in DMSO with salts dissolved in water (water content<10%) with a final peptide concentration of 20 mg/ml. The reaction, complete after 5 min, was monitored by UPLC analysis on a BEH300 C4 Acquity Waters column (2.1×100 mm, 1.7 μm) with a gradient of 30% B-30% B (1 min), 30% B-50% B (4 min), 50% B-90% B (0.2 min) (eluents: A=H2O+0.1% TFA; B=CH3CN+0.1% TFA); flow: 0.4 mL/min; λ: 214 nm; temp: 45° C.; MS: Waters Acquity ESI+, single quadrupole. The reaction was quenched with TFA, diluted with DMSO and loaded on a reversed-phase HPLC using a Delta Pak C4 25×200 mm, 300 A, 15 μm column and the linear gradient: 25% B-25% B (5 min), 25% B-40% B in 20 min (eluents: A=H2O+0.1% TFA; B=CH3CN+0.1% TFA; Flow: 50 mL/min; λ: 214 n). The analytical characterization was performed on a BEH300 C4 Acquity Waters 2.1×100 mm, 1.7 μm column with a gradient of 30% B-30% B (1 min), 30% B-50% B (5 min), 50% B-90% B (0.2 min); flow: 0.4 mL/min; λ: 214 nm; temp: 45° C.; MS: Waters Acquity ESI+, single quadrupole.

For PepSP1344, the dimeric precursor PepSP1343 was incubated with 1.1 eq of DBCO-PEG4-NHS ester (BroadPharm, MW: 649.7 Da) and 1% DIPEA was added. The reaction was performed in DMSO with a final peptide concentration of 20 mg/ml. The reaction, complete after 1 h, was monitored by UPLC analysis on a BEH300 C4 Acquity Waters column (2.1×100 mm, 1.7 μm) with a gradient of 35% B-35% B (1 min), 35% B-55% B (4 min), 55% B-90% B (0.2 min) (eluents: A=H2O+0.1% TFA; B=CH3CN+0.1% TFA); flow: 0.4 mL/min; λ: 214 nm; temp: 45° C.; MS: Waters Acquity ESI+, single quadrupole. The reaction was quenched with TFA, diluted with DMSO and loaded on a reversed-phase HPLC using a Waters Delta Pak C4 (200×25 mm 300 A 15 um) column and the linear gradient: 25% B-40% B in 20 min (eluents: A=H2O+0.1% TFA; B=CH3CN+0.1% TFA; Flow: 50 mL/min; λ: 214 nm). The analytical characterization was performed on a BEH300 C4 Acquity Waters (2.1×100 mm, 1.7 μm) column with a gradient of 35% B-35% B (1 min), 35% B-55% B (4 min), 55% B-90% B (0.2 min) (eluents: A=H2O+0.1% TFA; B=CH3CN+0.1% TFA); flow: 0.4 mL/min; λ: 214 nm; temp: 45° C.; MS: Waters Acquity ESI+, single quadrupole. Similarly, PepSP1371 was synthesized using PepSP1343 as the precursor and BCN-PEG3-NHS ester (BroadPharm, MW: 494.53 Da).

Further work was performed to exploit synthetic new schemes that can be used as an alternative route to Scheme I outlined above. The new scheme, Scheme II, is compatible with disulfide bridge formation and conjugation to radioligands. First, precursor compounds, PepSP1321 and PepSP1324, were synthesized using Scheme II, and both have the spacer Gly-Gly-Ttds at the C terminus that can have the benefit of more flexibility to the final dimers. Other precursor compounds PepSP1396 and PepSP1342 were also synthesized that have the linker GG-Ttds-K(PEG4)-CONH2 and GG-Ttds-K(Ttds-Ttds)-CONH2, respectively.

The precursor compounds were used to synthesize compounds PepSP1462, PepSP1487, PepSP1488, and PepSP1489, which can be conjugated to a radioisotope such as 18F. Specifically, PepSP1462 was prepared using PepSP1324 and the reagent PEG4-DBCO; PepSP1487 was prepared using PepSP1324 and the NHS-Tri-functionalized linker (Scheme II); PepSP1488 was prepared using PepSP1396 and the NHS-Tri-functionalized linker (Scheme II); and PepSP1489 was prepared using PepSP1342 and the NHS-Tri-functionalized linker (Scheme II).

Specifically, for PepSP1462, the precursor PepSP1324 (1 eq) was dissolved in DMSO and 10 eq. of DIPEA were added to the solution. Then, 1.1 eq. of DBCO-PEG4-NHS (BroadPharm, MW: 649.7 Da) were dissolved in DMSO and slowly added dropwise to the solution containing the peptide precursor resulting in a final concentration of peptide of 30 mg/mL. The reaction was monitored by UPLC-MS analysis on a BEH300 C4 Acquity Waters column (2.1×100 mm, 1.7 μm) with a gradient of 30% B-30% B (1 min), 30% B-50% B (4 min), 50% B-90% B (0.2 min) (Eluents: A=H2O+0.1% TFA; B=CH3CN+0.1% TFA); Flow: 0.4 mL/min; λ: 214 nm; Temp: 45° C.; MS: Waters Acquity ESI+, single quadrupole. After 30 minutes the reaction was completed and loaded on a reversed-phase HPLC using a DeltaPak C4, 40×200 mm, 300 Å, 15 μm column and the linear gradient: 15% B-15% B (5 min), 15% B-35% B (20 min), 35% B-40% B (5 min) (Eluents: A=H2O+0.05% NH3; B=CH3CN; Flow: 80 mL/min; λ: 214 nm). The analytical characterization was performed on a BEH300 C4 Acquity Waters 2.1×100 mm, 1.7 μm column with a gradient of 30% B-30% B (1 min), 30% B-50% B (4 min), 50% B-90% B (0.2 min); flow: 0.4 mL/min; λ: 214 nm; temp: 45° C.; MS: Waters Acquity ESI+, single quadrupole.

For PepSP1487, 1488 and 1489, the precursors PepSP1324, 1396 and 1342, respectively, (1 eq.) were dissolved in DMSO and 10 eq. of DIPEA were added to the solution. Then, 0.5 eq. of NHS-Tri-functionalized linker (Scheme 4) were dissolved in DMSO and slowly added dropwise to the solution containing the peptide precursor resulting in a final concentration of peptide of 1 mg/mL. The reaction is monitored by UPLC-MS analysis on a BEH300 C4 Acquity Waters column (2.1×100 mm, 1.7 μm) with a gradient of 30% B-30% B (1 min), 30% B-50% B (4 min), 50% B-90% B (0.2 min) (Eluents: A=H2O+0.1% TFA; B=CH3CN+0.1% TFA); Flow: 0.4 mL/min; λ: 214 nm; Temp: 45° C.; MS: Waters Acquity ESI+, single quadrupole. After one night, the reaction is complete, it is diluted 1:2 with H2O+0.05% NH3 and loaded on a reversed-phase HPLC using a XBridge Protein BEH C4, 30×150 mm, 300 A, 5 μm column and the linear gradient: 5% B-5% B (5 min)-5% B-25% B (20 min), 25% B-30% B (5 min) (Eluents: A=H2O+0.05% NH3; B=CH3CN; Flow: 80 mL/min; λ: 214 nm). The analytical characterization was performed on a BEH300 C4 Acquity Waters 2.1×100 mm, 1.7 μm column with a gradient of −30% B-30% B (1 min), −30% B-50% B (4 min), −50% B-90% B (0.2 min); flow: 0.4 mL/min; λ: 214 nm; temp: 45° C.; MS: Waters Acquity ESI+, single quadrupole.

The DBCO derivatives PepSP1462, PepSP1487, PepSP1488 and PepSP1489 were finally conjugated with the Fluoroethylazide (FEA) to obtain the final fluoro-labelled compounds PepSP1538, PepSP1581, PepSP1582 and PepSP1583, respectively, as follows.

Synthesis of monomeric Fluoroethylazide (FEA) derivative PepSP1538: 1 eq of the DBCO peptide precursor, PepSP1462, was dissolved in DMSO at a final concentration of 20 mg/ml and 1.2 eq of FEA were added to the reaction mixture. The reaction was monitored by UPLC-MS analysis on a BEH300 C4 Acquity Waters column (2.1×100 mm, 1.7 μm) with a gradient of 30% B-30% B (1 min), 30% B-60% B (4 min), 60% B-90% B (0.2 min) (Eluents: A=H2O+0.1% TFA; B=CH3CN+0.1% TFA); Flow: 0.4 mL/min; λ: 214 nm; Temp: 45° C.; MS: Waters Acquity ESI+, single quadrupole. After the reaction was complete, it was diluted 1:2 with H2O+0.05% NH3 and loaded on a reversed-phase HPLC using a C4, 25×200 mm, 15 μm, 300 A Waters Deltapak column and the linear gradient: 20% B-20% B (5 min) to 35% B (20 min) (Eluents: A=H2O+0.05% NH3; B=CH3CN; Flow: 80 mL/min; λ: 214 nm). The analytical characterization was performed on a BEH300 C4 Acquity Waters 2.1×100 mm, 1.7 μm column with a gradient of 30% B-30% B (1 min), 30% B-60% B (4 min), 60% B-90% B (0.2 min); eluents: A: H2O+0.1% TFA; B: AcN+0.1% TFA; flow: 0.4 mL/min; λ: 214 n; temp: 45° C.; MS: Waters Acquity ESI+, single quadrupole.

Synthesis of dimeric FEA derivatives PepSP1581, PepSP1582 and PepSP1583: 1 eq of the dimeric DBCO peptide precursor (PepSP1487, 1488 or 1489) was dissolved in DMSO at a final concentration of 1 mg/ml, and 1.2 eq of FEA were added to the reaction mixture. The reaction was monitored by UPLC-MS analysis on a BEH300 C4 Acquity Waters column (2.1×100 mm, 1.7 μm) with a gradient of 30% B-30% B (1 min), 30% B-60% B (4 min), 60% B-90% B (0.2 min) (Eluents: A=H2O+0.1% TFA; B=CH3CN+0.1% TFA); Flow: 0.4 mL/min; λ: 214 n; Temp: 45° C.; MS: Waters Acquity ESI+, single quadrupole. After the reaction was complete, it was diluted 1:2 with H2O+0.05% NH3 and loaded on a reversed-phase HPLC using a C4, 20×150 mm, 5 μm, 300 A Waters XBridge column and the linear gradient: 10% B-10% B (5 min) to 25% B (20 min) (Eluents: A=H2O+0.05% NH3; B=CH3CN; Flow: 80 mL/min; λ: 214 nm). The analytical characterization was performed on a BEH300 C4 Acquity Waters 2.1×100 mm, 1.7 μm column with a gradient of 30% B-30% B (1 min), 30% B-60% B (4 min), 60% B-90% B (0.2 min); eluents: A: H2O+0.1% TFA; B: AcN+0.1% TFA; flow: 0.4 mL/min; λ: 214 nm; temp: 45° C.; MS: Waters Acquity ESI+, single quadrupole.

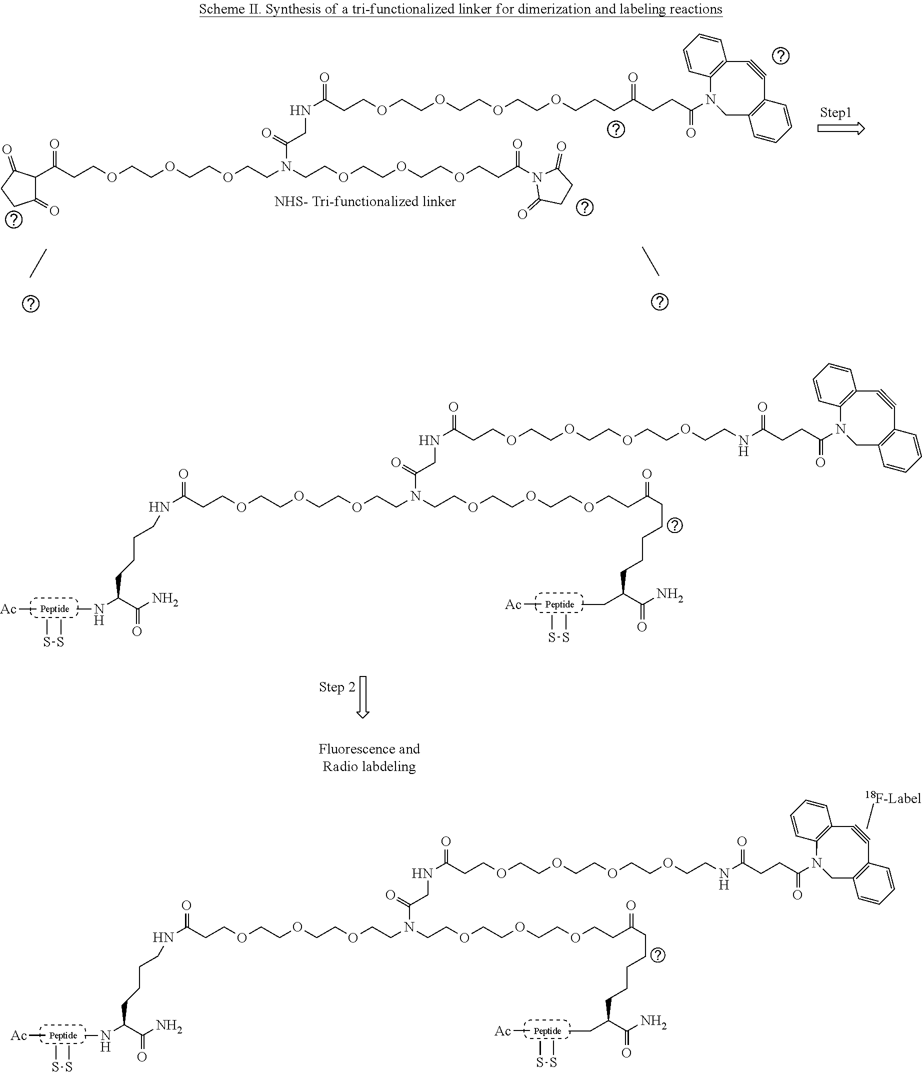

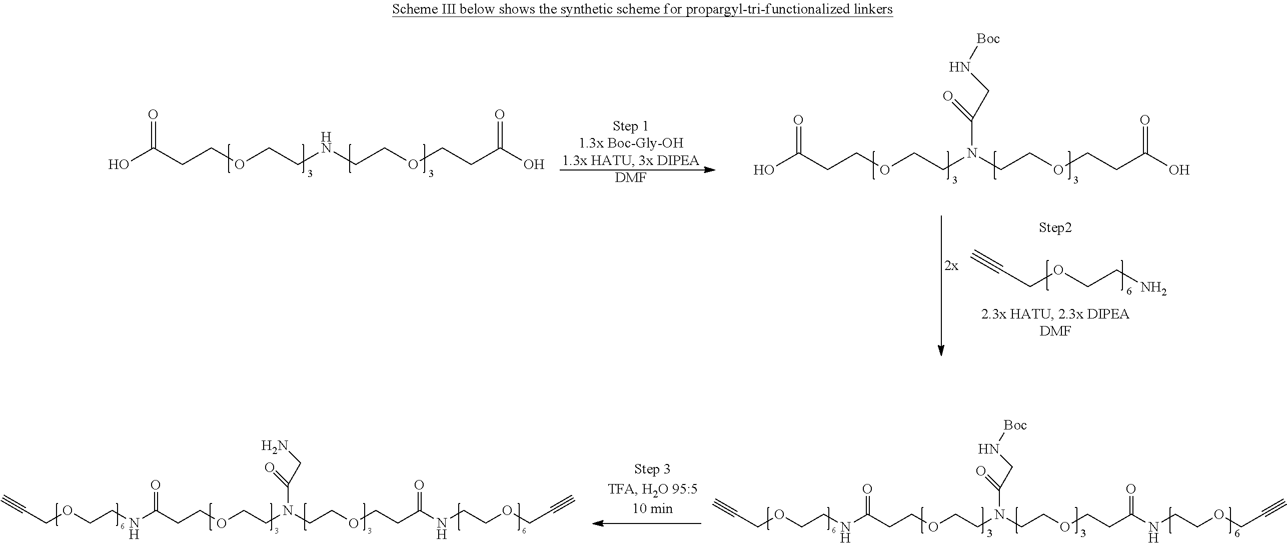

The following steps were performed to synthesize the propargyl-tri-functionalized linkers (Scheme III). Step 1: 1.3 eq of Boc-Gly-OH (4530-20-5) were dissolved in DMF with 1.3 eq of HATU, and after 5 min the mixture was added to 1 eq of NH-bis(PEG4-acid), dissolved in DMF, and DIPEA (3 eq). The coupling reaction was complete after 15 min. Reaction monitoring was done by UPLC-MS. Step 2: HATU and DIPEA (2.3 eq) were added to the reaction mixture, stirred at room temperature and after 5 min, 2 eq of propargyl-PEGx-amine were added, dissolved in DMF. The reaction was stirred and monitored by UPLC-MS, then quenched with AcOH and concentrated to dryness under high vacuum. Step 3: Crude material was dissolved in TFA/H2O 95:5 and stirred at room temperature for 10 min, then concentrated to dryness. The crude material was purified by RP Flash chromatography using a Luknova C18 column (Gradient: (% B): 0% for 4 CV, 0% to 35% in 10 CV, 35% for 3 CV. A: H2O+0.1% TFA; B: ACN+0.1% TFA; λ: 214 nm; collected fraction were lyophilized. UPLC-MS: Acquity BEH C18, 2.1×100 mm, 1.7 um, 130 A Flow: 0.4 mL/min; Gradient (% B): 20% in 1 min, 20% to 70% in 4 min; A: H2O+0.1% TFA; B: ACN+0.1% TFA; λ: 214 n).

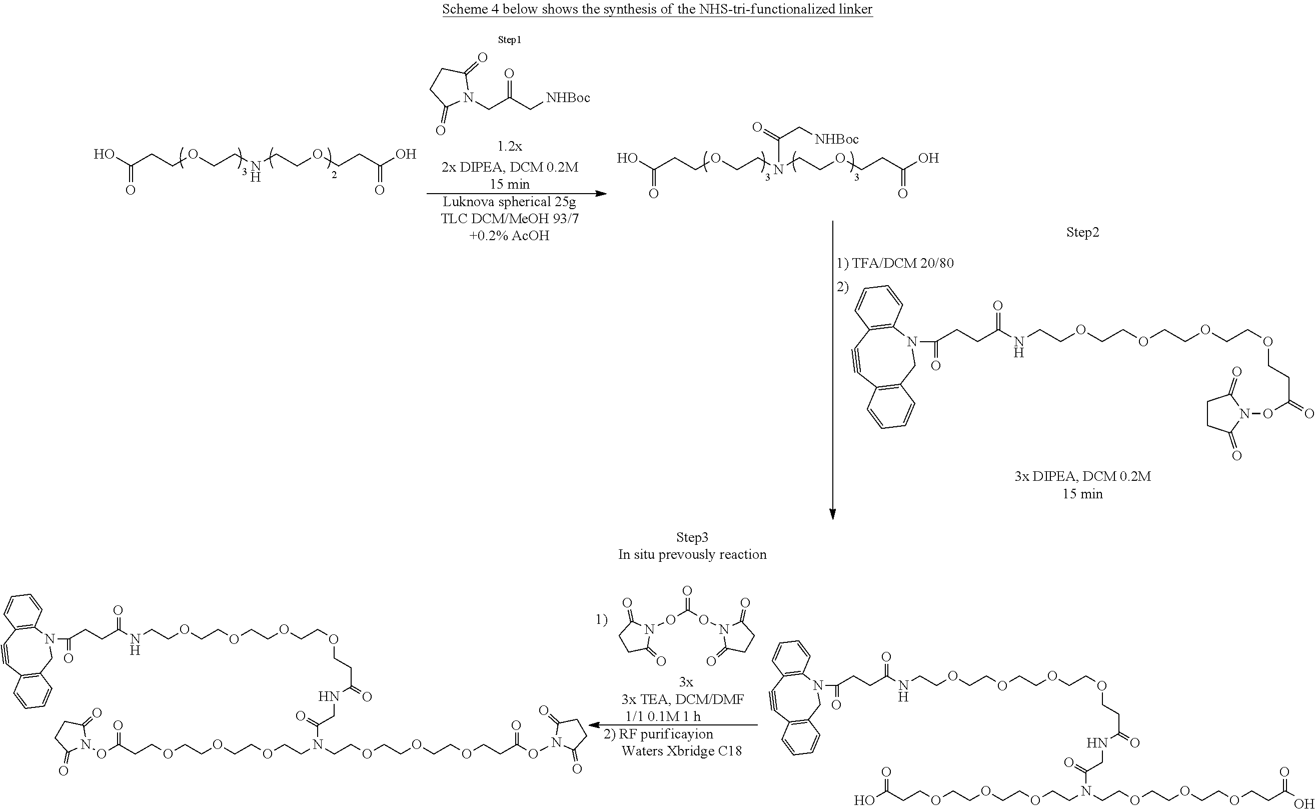

The following steps were performed to synthesize the NHS-tri-functionalized linker (Scheme 4), Step 1: 1 eq of NH-bis(PEG3-acid) HCl salt (BroadPharm, 425.47 Da) and 1.2 eq of Boc-Gly-OSu (3392-07-2) were dissolved in DCM and 2 eq of DIPEA were added; the reaction was monitored by UPLC-MS and was complete after 15 min. It was purified by flash chromatography using a Luknova SuperSep HP 25 μg column (Gradient: (% B): 0% for 2 CV, 0% to 15% in 8 CV, 15% for 2 CV. A: DCM+0.2% acetic acid; B: MeOH+0.2% acetic acid; λ: 206 nm; collected fractions were lyophilized. UPLC-MS: Acquity BEH C18, 2.1×100 mm, 1.7 um, 130 A Flow: 0.4 mL/min; Gradient (% B): 5% B-5% B (1 min), 5% B-95% B (4 min); A: H2O+0.1% TFA; B: CH₃CN+0.1% TFA; λ: 214 nm). Step 2: Purified product was dissolved in TFA/DCM 20:80, stirred for 10 minutes and then concentrated to dryness. The product was dissolved in DCM, 3 eq of DIPEA were added and then 1 eq of DBCO-PEG4-NHS ester (BroadPharm, MW: 649.7 Da) was added and stirred for 15 minutes and monitored by UPLC-MS. Step 3: 3 eq of TEA were added to 1 eq of the product dissolved in DCM; 3 eq of N,N′-Disuccinimidyl carbonate (BroadPharm, 256.17 Da) dissolved in DMF were added in order to have the reaction in DCM:DMF 1:1; the reaction was stirred for 1 h and monitored by UPLC-MS. It was loaded on a reversed-phase HPLC and purified using a Waters XBridge C18 (50×150 mm, 130 A, 5 μm) column and the linear gradient: 30% B-30% B (5 min), 30% B-50% B (20 min); eluents: A=H2O+0.1% TFA; B=CH3CN+0.1% TFA; flow: 80 mL/min; λ: 214 nm; collected fraction were lyophilized. The analytical characterization was performed on a Acquity BEH C18, 2.1×100 mm, 1.7 um, 130 A Flow: 0.4 mL/min; Gradient (% B): 5-B-5% B (1 min), 5-B-95% B (4 min); A: H2O+0.1% TFA; B: CH₃CN+0.1% TFA; λ: 214 nm; temp: 45° C.; MS: Waters Acquity ESI+, single quadrupole.

The sequence and structure of the labeled and/or dimerized peptides are shown in Table 5 below.

TABLE 5
Sequence and structure of labeled and dimerized peptides

| PEP ID | Sequence & structure |
|---|---|
| PepSP1273 | AETVEFWGCTGTWGNETCWW-Ttds-K(N3)-CONH2 |
| | 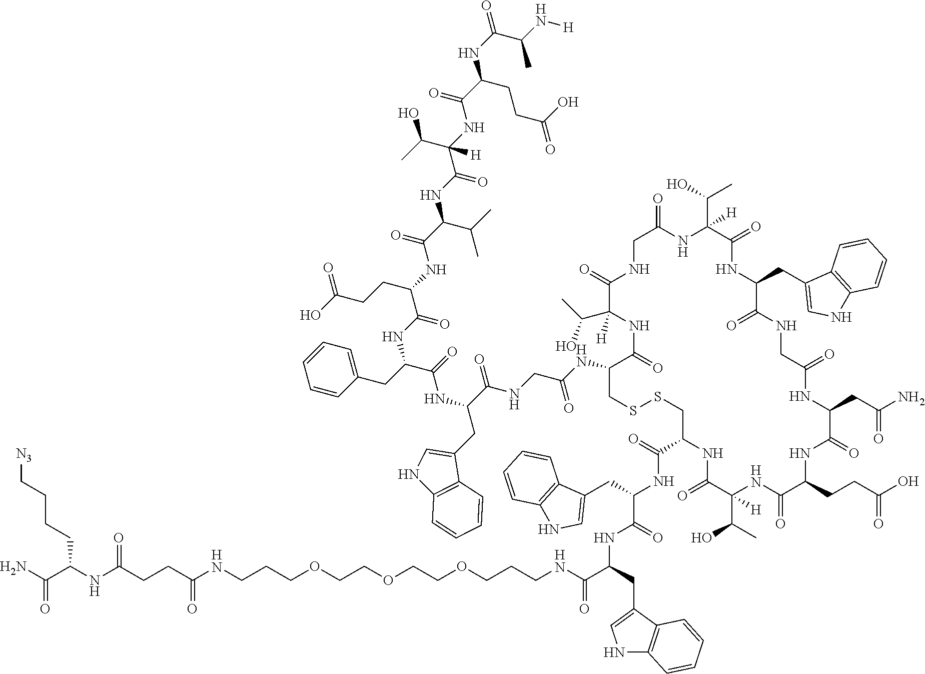 [SMILES: CC(C)[C@H](NC(=O)[C@@H](NC(=O)[C@H](CCC(=O)O)NC(=O)[C@H](C)N)[C@@H](C)O)C(=O)N[C@@H](CCC(=O)O)C(=O)N[C@@H](Cc1ccccc1)C(=O)N[C@@H](Cc1c[nH]c2ccccc12)C(=O)NCC(=O)N[C@H]1CSSC[C@@H](C(=O)N[C@@H](Cc2c[nH]c3ccccc23)C(=O)N[C@@H](Cc2c[nH]c3ccccc23)C(=O)NCCCOCCOCCOCCCNC(=O)CCC(=O)N[C@@H](CCCCN=[N+]=[N-])C(N)=O)NC(=O)[C@H]([C@@H](C)O)NC(=O)[C@H](CCC(=O)O)NC(=O)[C@H](CC(N)=O)NC(=O)CNC(=O)[C@H](Cc2c[nH]c3ccccc23)NC(=O)[C@H]([C@@H](C)O)NC(=O)CNC(=O)[C@H]([C@@H](C)O)NC1=O] |
| PepSP1215 | AETVEFWGCTGTWGNETCWW-Ttds-K(AlexaFluor647)-CONH2 |
| | 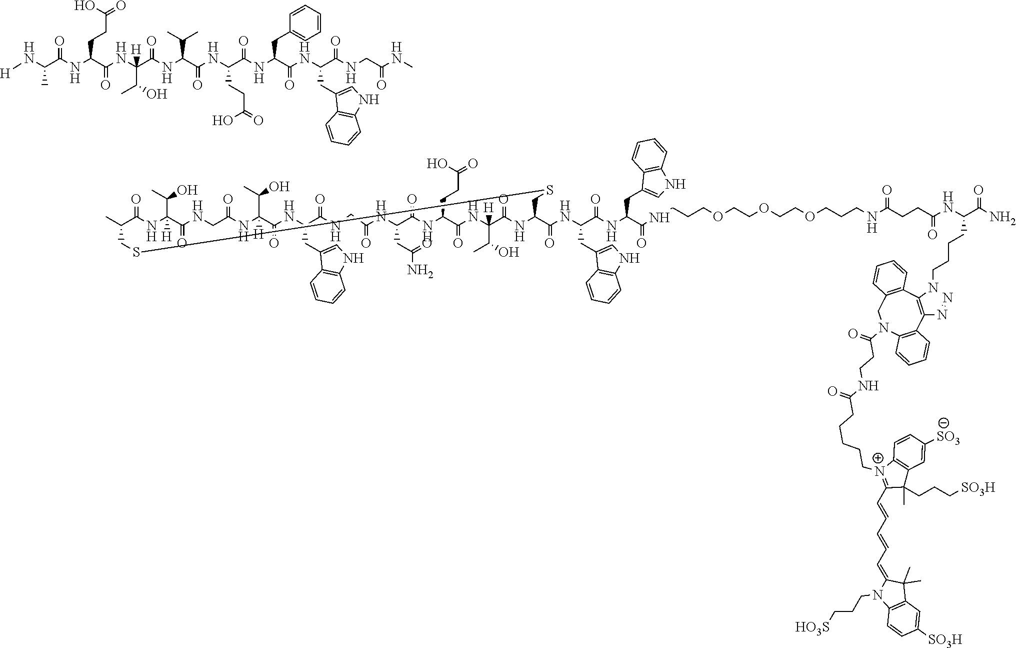 |
| PepSP1274 | AETVEFWWCNGNSENWTCTW-Ttds-K(N3)-CONH2 |
| | 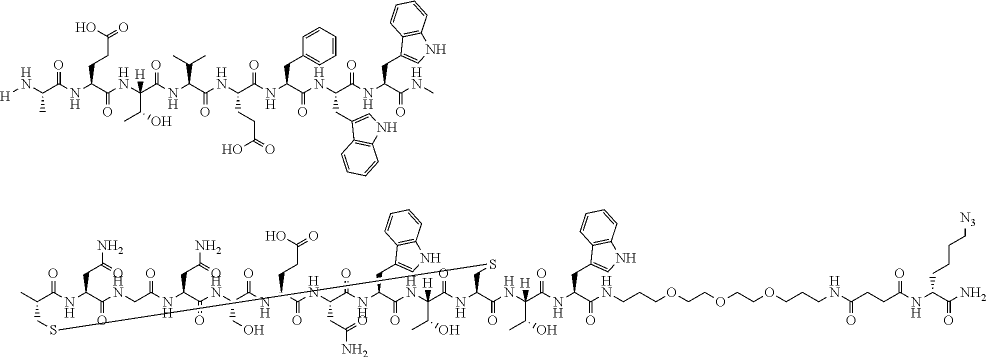 [SMILES: CNC(=O)[C@H](Cc1c[nH]c2ccccc12)NC(=O)[C@H](Cc1c[nH]c2ccccc12)NC(=O)[C@H](Cc1ccccc1)NC(=O)[C@H](CCC(=O)O)NC(=O)[C@@H](NC(=O)[C@@H](NC(=O)[C@H](CCC(=O)O)NC(=O)[C@H](C)N)[C@@H](C)O)C(C)C ; C[C@H]1CSSC[C@@H](C(=O)N[C@H](C(=O)N[C@@H](Cc2c[nH]c3ccccc23)C(=O)NCCCOCCOCCOCCCNC(=O)CCC(=O)N[C@H](CCCCN=[N+]=[N-])C(N)=O)[C@@H](C)O)NC(=O)[C@H]([C@@H](C)O)NC(=O)[C@H](Cc2c[nH]c3ccccc23)NC(=O)[C@H](CC(N)=O)NC(=O)[C@H](CCC(=O)O)NC(=O)[C@H](CO)NC(=O)[C@H](CC(N)=O)NC(=O)CNC(=O)[C@H](CC(N)=O)NC1=O] |
| PepSP1216 | AETVEFWWCNGNSENWTCTW-Ttds-K(AlexaFluor647)-CONH2 |
| | 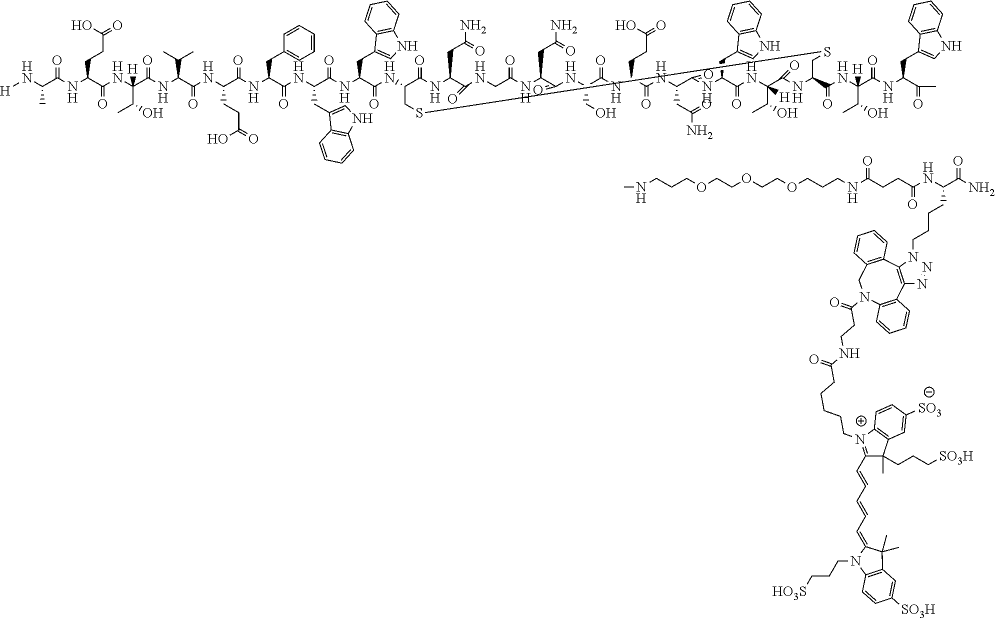 |
| PepSP1270 | [AETVEFWGCTGTWGNETCWW-Ttds-K(triazole)-CONH2]2-bisPEG₆ |
| | 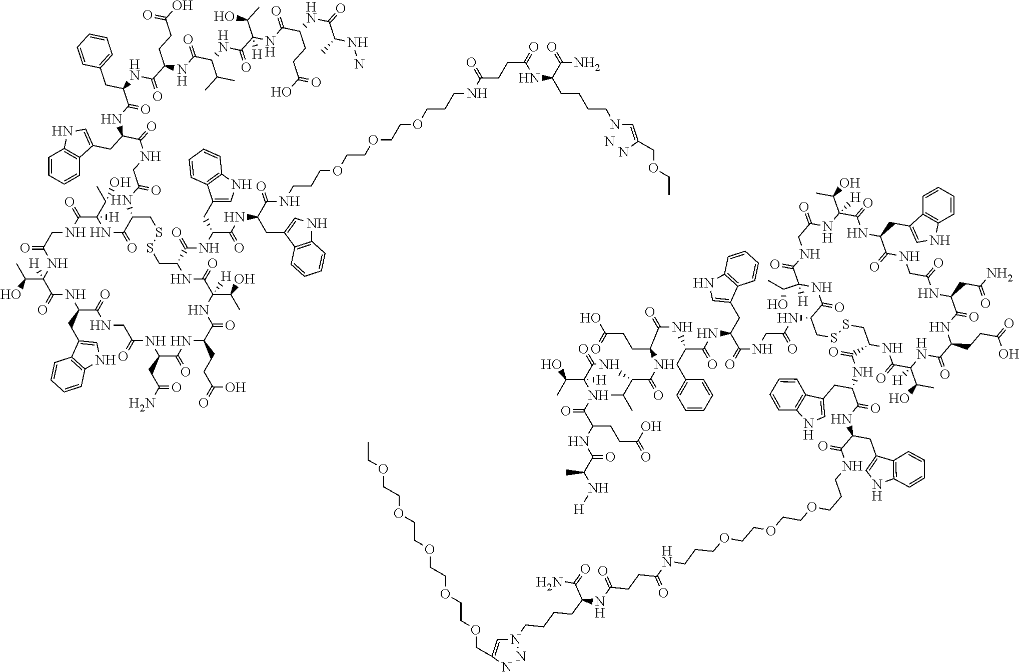 |
| PepSP1271 | [AETVEFWGCTGTWGNETCWW-Ttds-K(triazole)-CONH2]2-bisPEG₁₄ |
| | 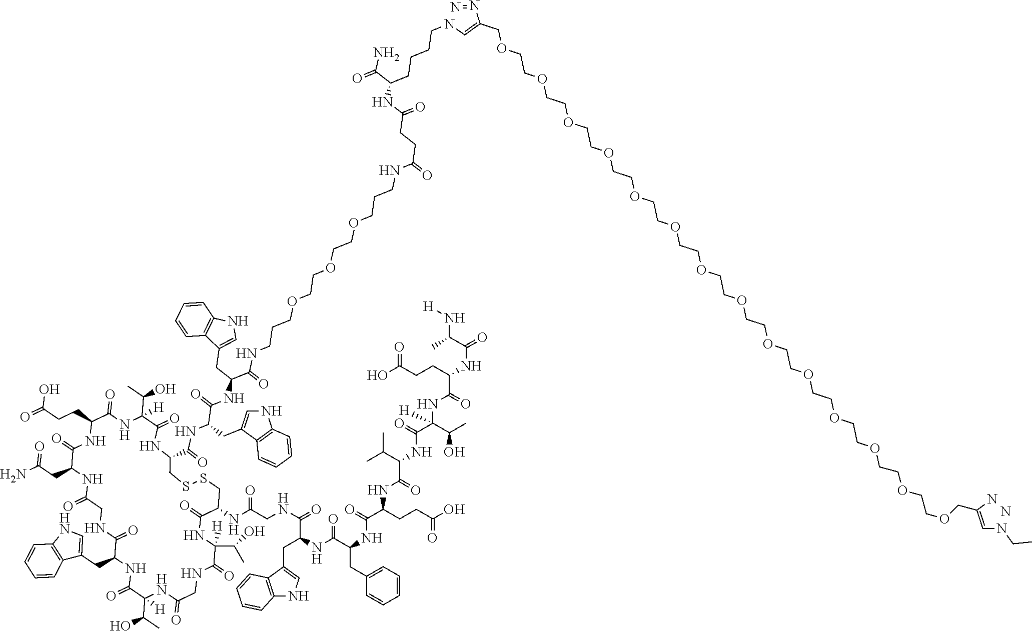 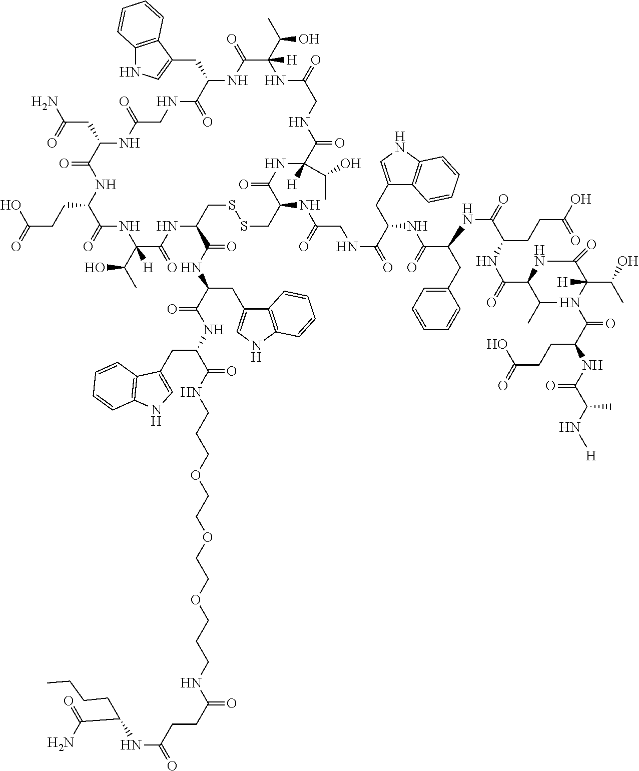 |
| PepSP1272 | [AETVEFWGCTGTWGNETCWW-Ttds-K(triazole)-CONH2]2-bisPEG₁₈ |
| | 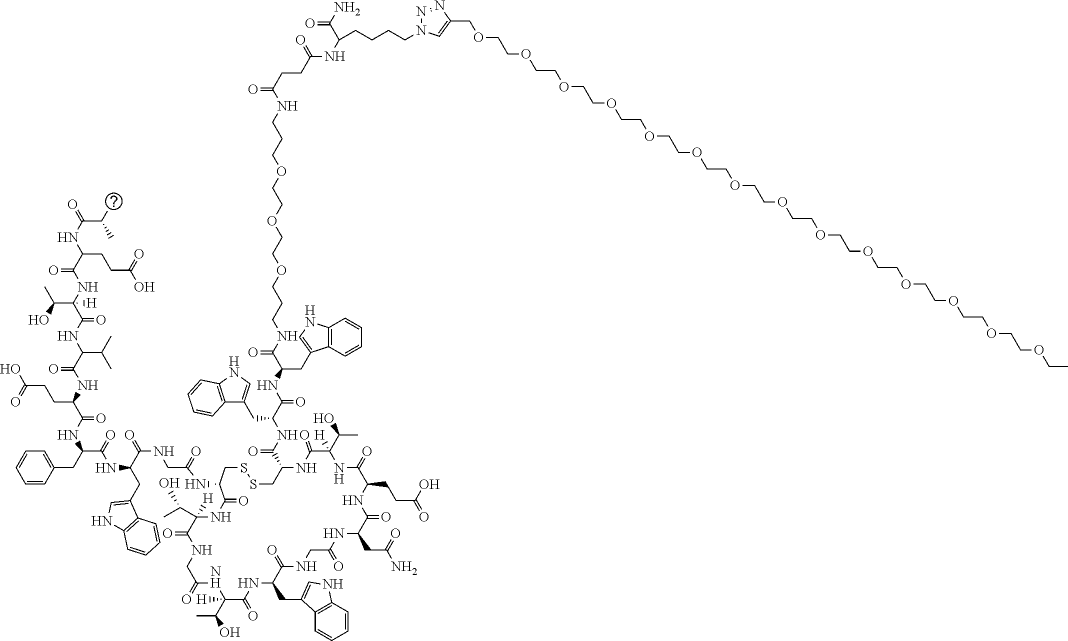 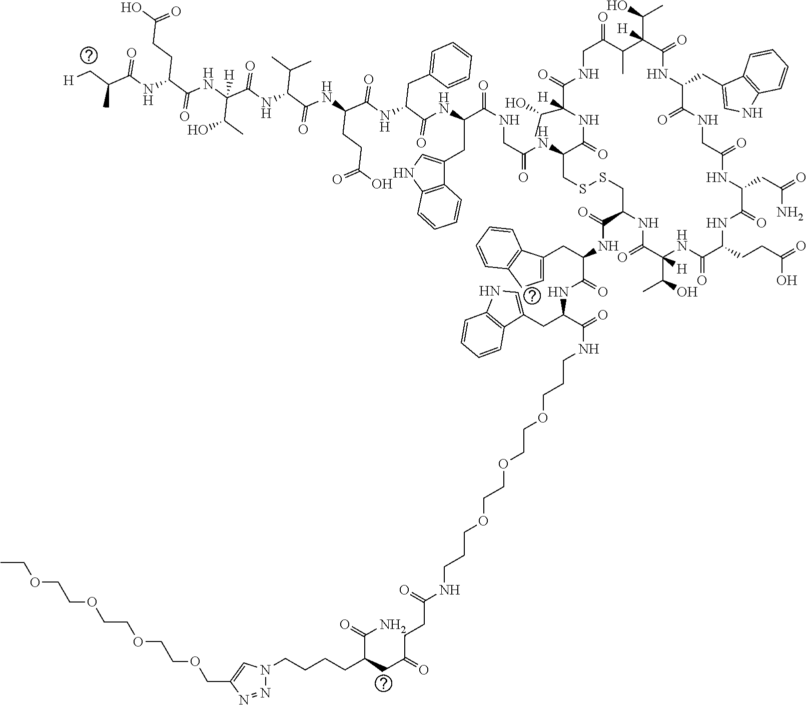 |
| PepSP1318 | Ac-AETVEFWGCTGTWGNETCWW-Ttds-K(N3)-CONH2 |
| | 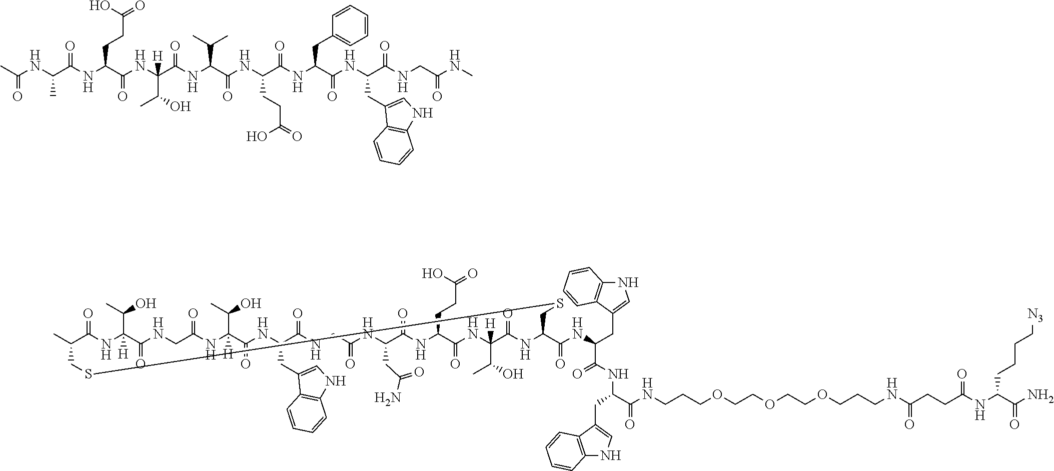 [SMILES: CNC(=O)CNC(=O)[C@H](Cc1c[nH]c2ccccc12)NC(=O)[C@H](Cc1ccccc1)NC(=O)[C@H](CCC(=O)O)NC(=O)[C@@H](NC(=O)[C@@H](NC(=O)[C@H](CCC(=O)O)NC(=O)[C@H](C)NC(C)=O)[C@@H](C)O)C(C)C ; C[C@H]1CSSC[C@@H](C(=O)N[C@@H](Cc2c[nH]c3ccccc23)C(=O)N[C@@H](Cc2c[nH]c3ccccc23)C(=O)NCCCOCCOCCOCCCNC(=O)CCC(=O)N[C@H](CCCCN=[N+]=[N-])C(N)=O)NC(=O)[C@H]([C@@H](C)O)NC(=O)[C@H](CCC(=O)O)NC(=O)[C@H](CC(N)=O)NC(=O)CNC(=O)[C@H](Cc2c[nH]c3ccccc23)NC(=O)[C@H]([C@@H](C)O)NC(=O)CNC(=O)[C@H]([C@@H](C)O)NC1=O] |
| PepSP1343 | [Ac-AETVEFWGCTGTWGNETCWW-Ttds-K(triazole-PEG6-PEG3)]2-CONH2 |
| | 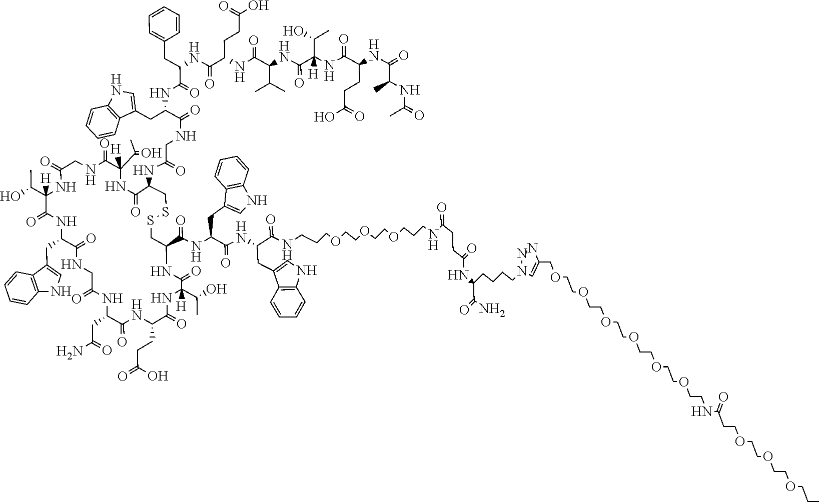 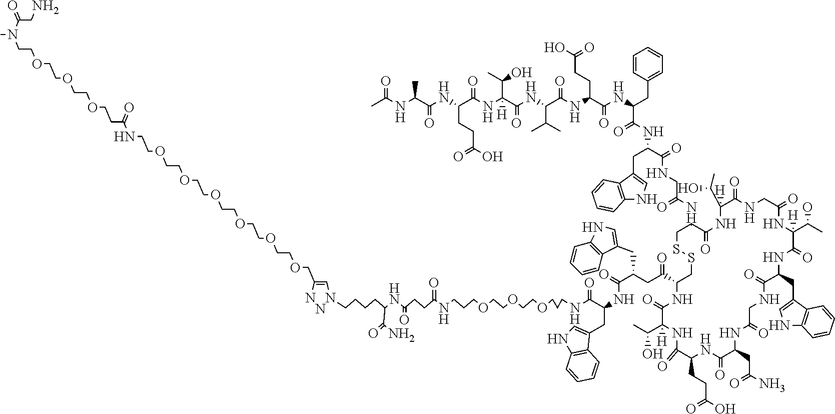 |
| PepSP1344 | [Ac-AETVEFWGCTGTWGNETCWW-Ttds-K(triazole-PEG6-PEG3)]2-DBCO |
| | 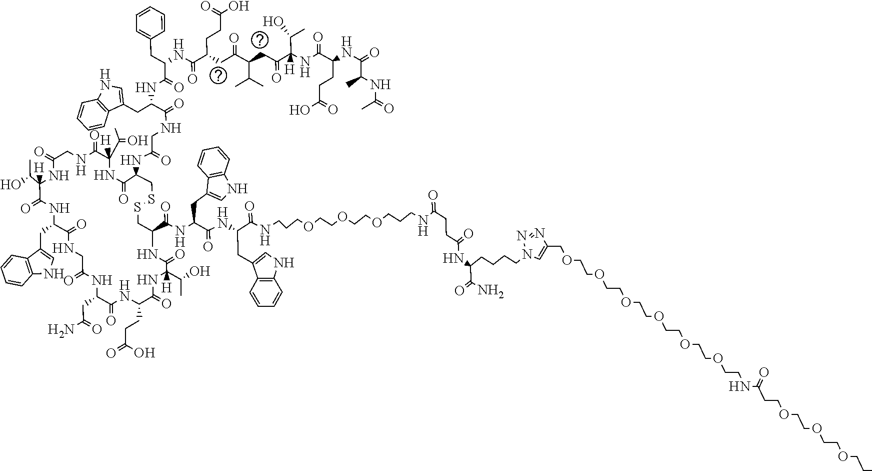 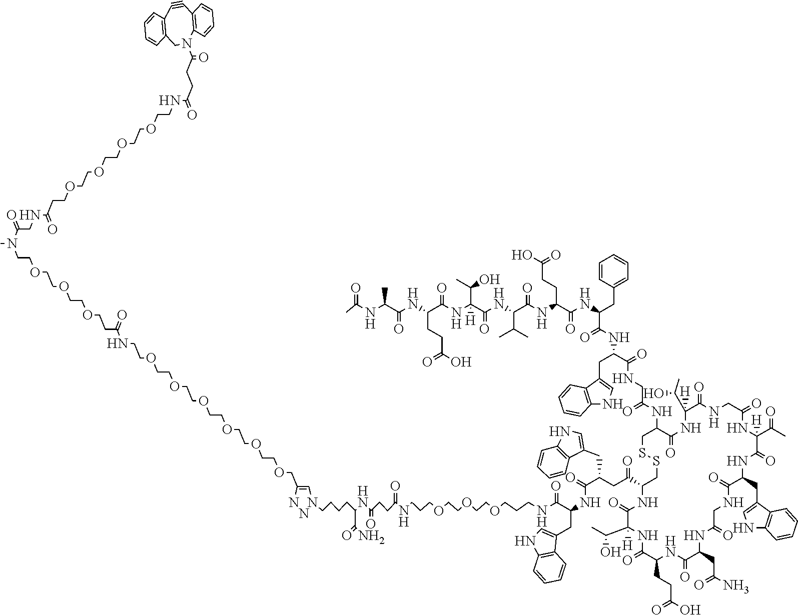 |
| PepSP1371 | [Ac-AETVEFWGCTGTWGNETCWW-Ttds-K(triazole-PEG6-PEG3)-CONH2]2-PEG3-BCN |
| | 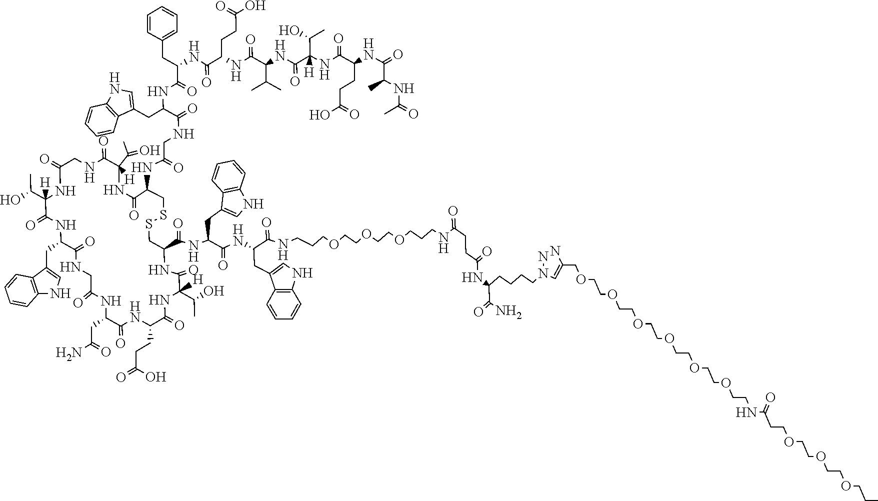 [SMILES: CCCOCCOCCOCCC(=O)NCCOCCOCCOCCOCCOCCOCc1cn(CCCC[C@H](NC(=O)CCC(=O)NCCCOCCOCCOCCCNC(=O)[C@H](Cc2c[nH]c3ccccc23)NC(=O)[C@H](Cc2c[nH]c3ccccc23)NC(=O)[C@@H]2CSSC[C@H](NC(=O)CNC(=O)C(Cc3c[nH]c4ccccc34)NC(=O)[C@H](Cc3ccccc3)NC(=O)[C@H](CCC(=O)O)NC(=O)[C@@H](NC(=O)[C@@H](NC(=O)[C@H](CCC(=O)O)NC(=O)[C@H](C)NC(C)=O)[C@@H](C)O)C(C)C)C(=O)N[C@@H](C(C)=O)C(=O)NCC(=O)N[C@@H]([C@@H](C)O)C(=O)N[C@@H](Cc3c[nH]c4ccccc34)C(=O)NCC(=O)N[C@@H](CC(N)=O)C(=O)N[C@@H](CCC(=O)O)C(=O)N[C@@H]([C@@H](C)O)C(=O)N2)C(N)=O)nn1] 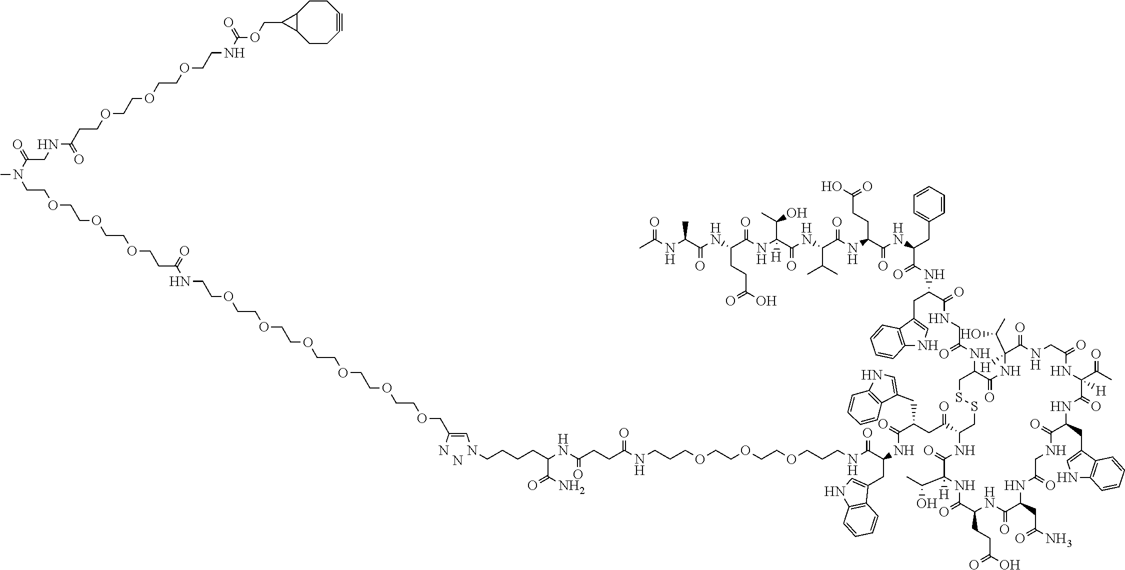 |
| PepSP1384 | [AETVEFWWCNGNSENWTCTW-Ttds-K(triazole)-CONH2]2-bisPEG18 |
| | 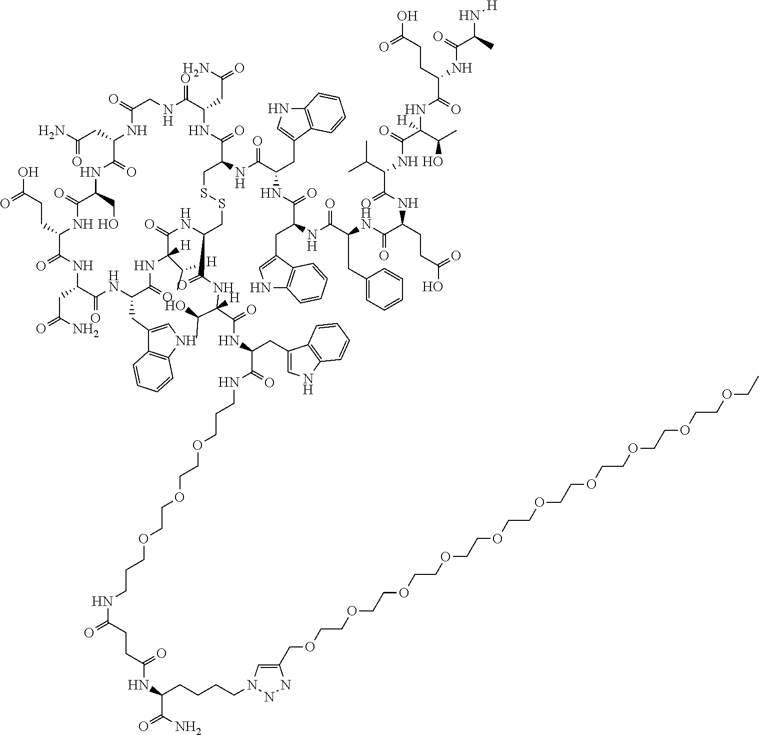 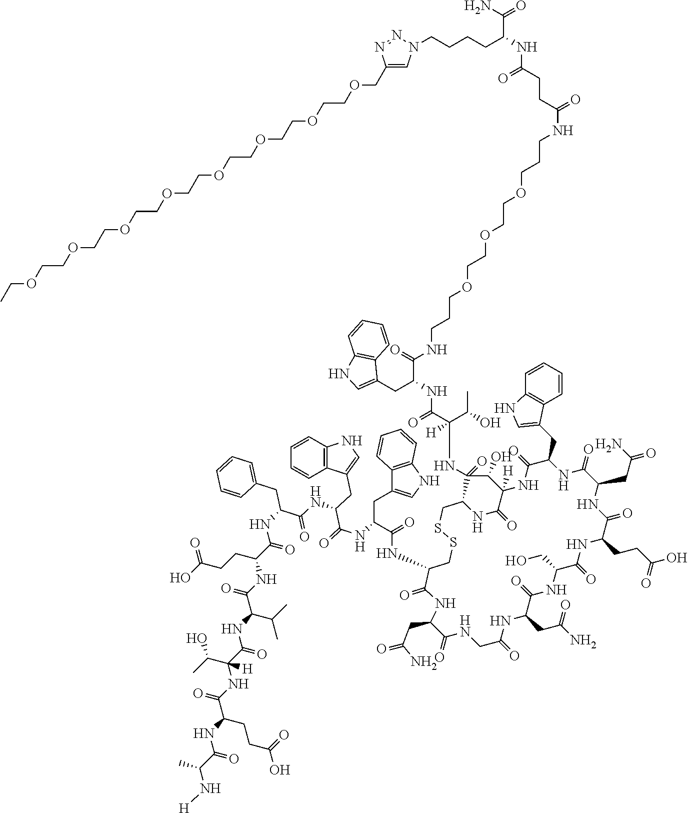 |
| PepSP1321 | Ac-AETVEFWGCTGTWGNETCWW-GG-Ttds-K-CONH2 |
| | 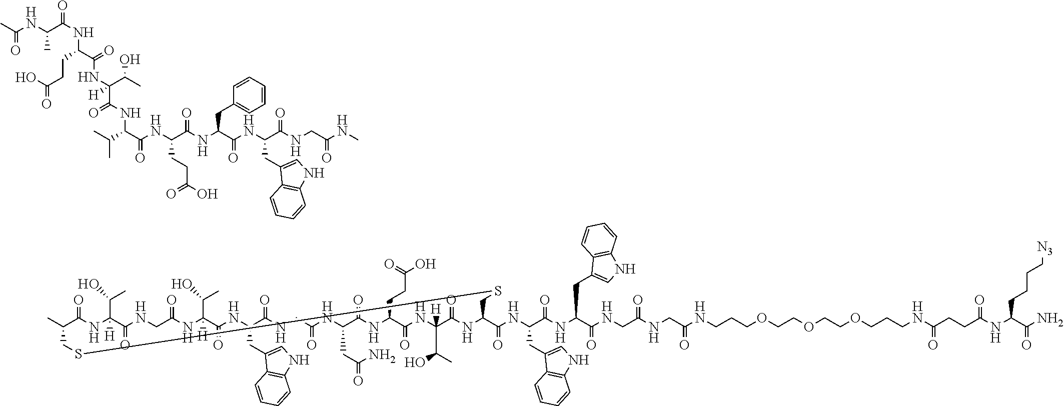 [SMILES: CNC(=O)CNC(=O)[C@H](Cc1c[nH]c2ccccc12)NC(=O)[C@H](Cc1ccccc1)NC(=O)[C@H](CCC(=O)O)NC(=O)[C@@H](NC(=O)[C@@H](NC(=O)[C@H](CCC(=O)O)NC(=O)[C@H](C)NC(C)=O)[C@@H](C)O)C(C)C ; C[C@H]1CSSC[C@@H](C(=O)N[C@@H](Cc2c[nH]c3ccccc23)C(=O)N[C@@H](Cc2c[nH]c3ccccc23)C(=O)NCC(=O)NCC(=O)NCCCOCCOCCOCCCNC(=O)CCC(=O)N[C@@H](CCCCN=[N+]=[N-])C(N)=O)NC(=O)[C@H]([C@@H](C)O)NC(=O)[C@H](CCC(=O)O)NC(=O)[C@H](CC(N)=O)NC(=O)CNC(=O)[C@H](Cc2c[nH]c3ccccc23)NC(=O)[C@H]([C@@H](C)O)NC(=O)CNC(=O)[C@H]([C@@H](C)O)NC1=O] |
| PepSP1324 | Ac-AETVEFWWCNGNSENWTCTW-GG-Ttds-K-CONH2 |
| | 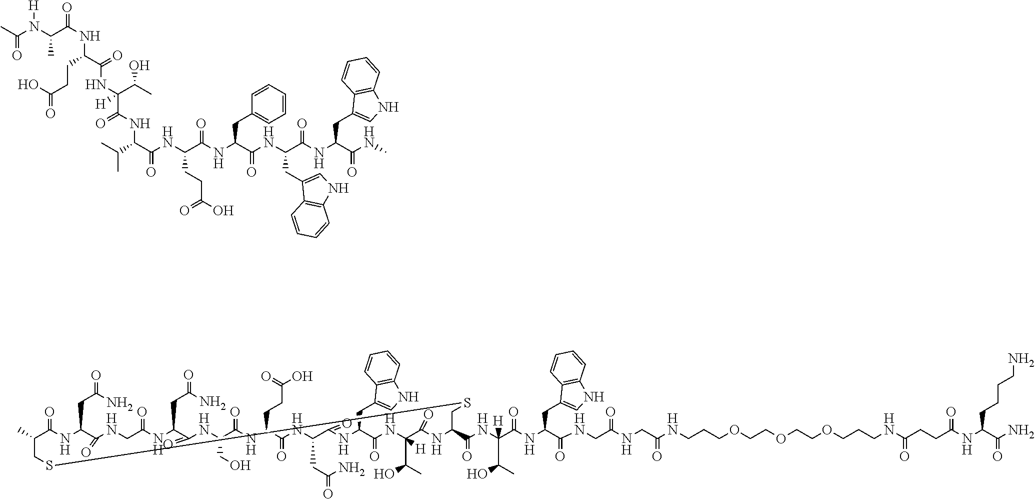 |
| PspSP1396 | Ac-AETVEFWWCNGNSENWTCTW-GG-Ttds-K(PEG4)-CONH2 |
| | 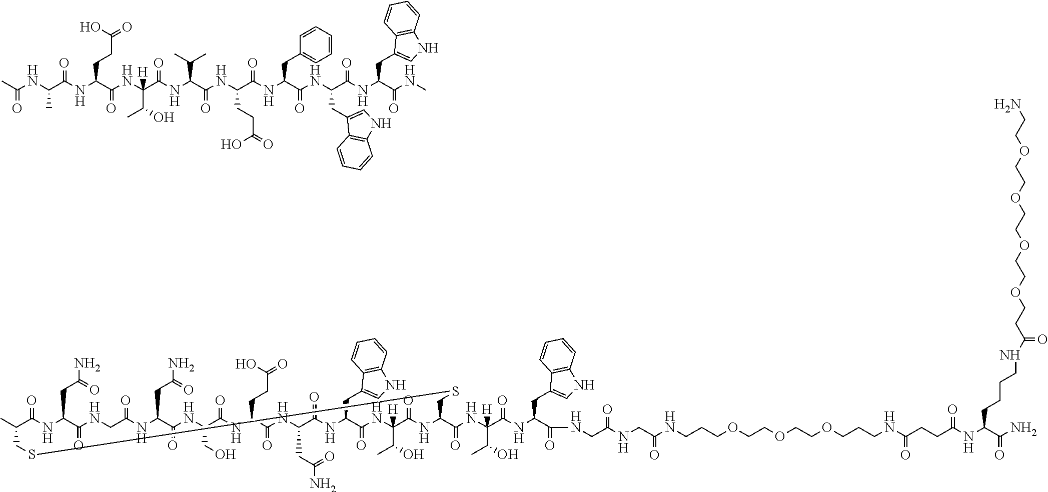 |
| PspSP1342 | Ac-AETVEFWWCNGNSENWTCTW-GG-Ttds-K(Ttds-Ttds)-CONH2 |
| | 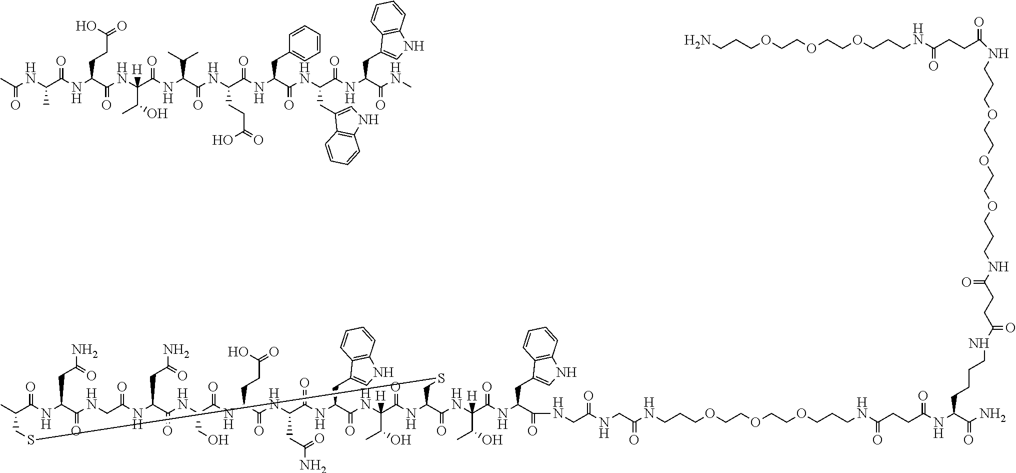 |
| PspSP1462 | Ac-AETVEFWWCNGNSENWTCTW-GG-Ttds-K(PEG4-DBCO)-CONH2 |
| | 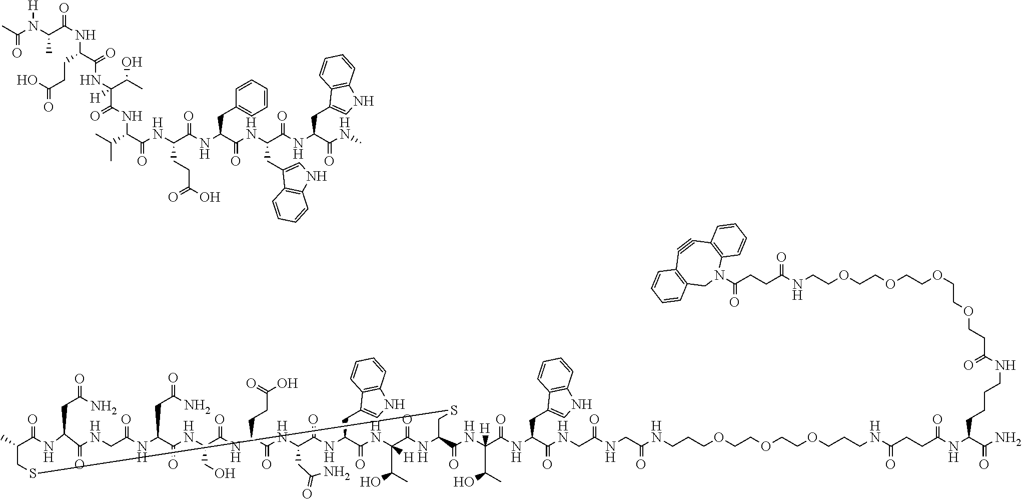 |
| PspSP1487 | [Ac-AETVEFWWCNGNSENWTCTW-GG-Ttds-K(PEG3)]2-PEG4-DBCO |
| | 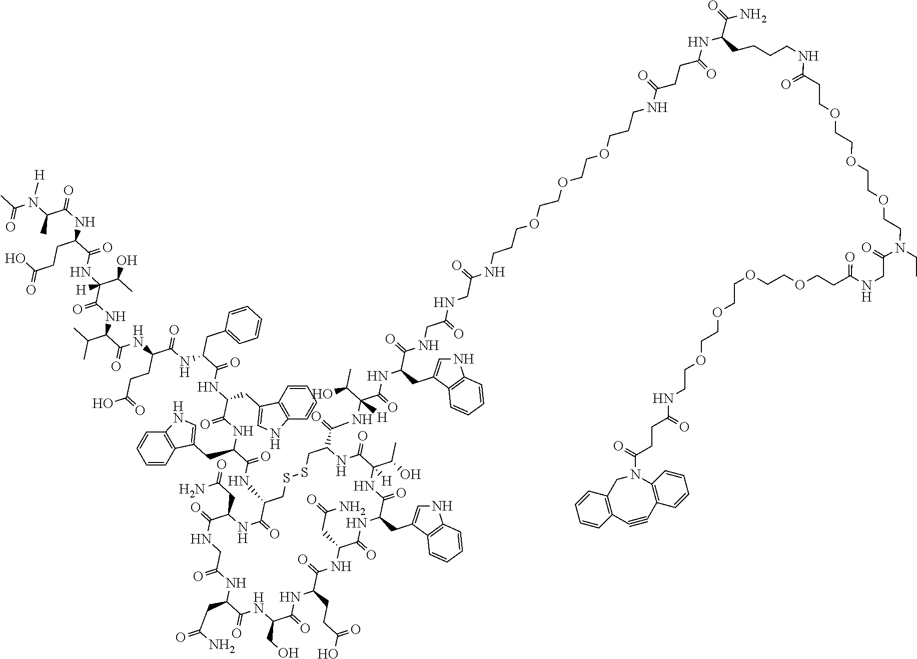 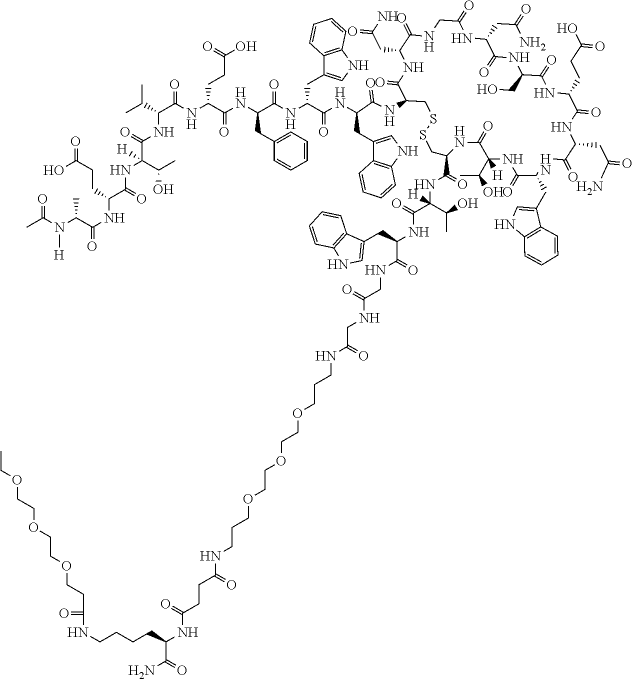 |
| PspSP1488 | [Ac-AETVEFWWCNGNSENWTCTW-GG-Ttds-K(PEG4-PEG3)]2-PEG4-DBCO |
| | 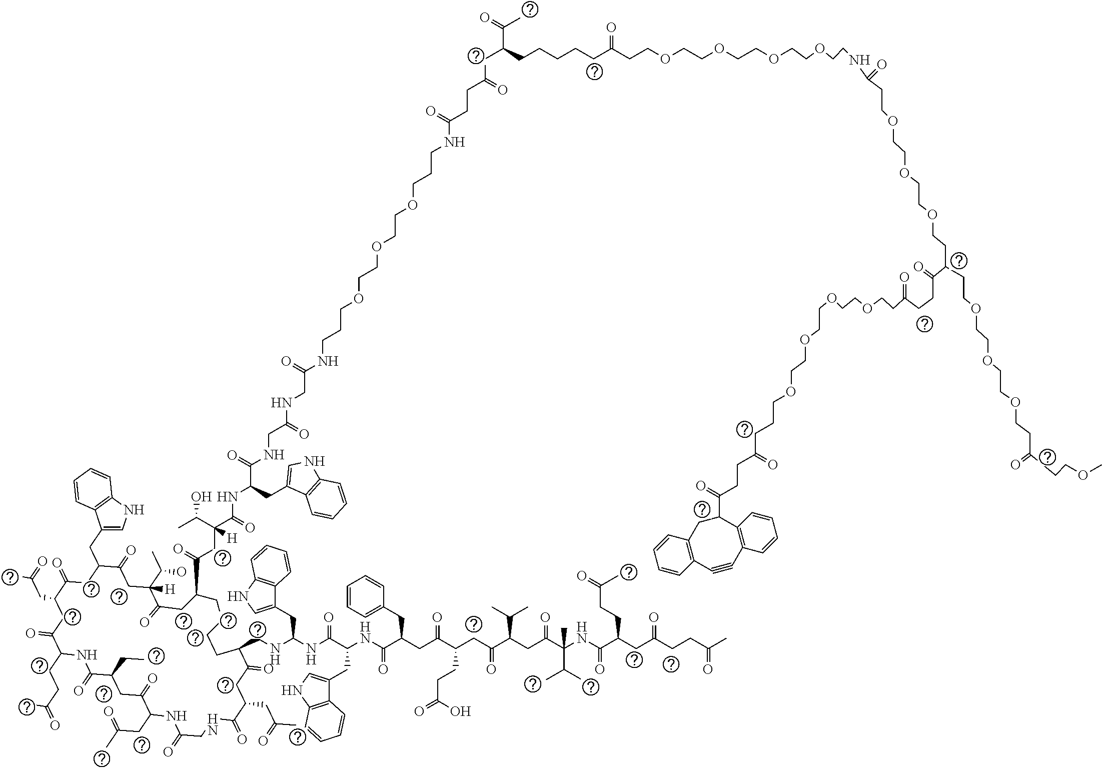 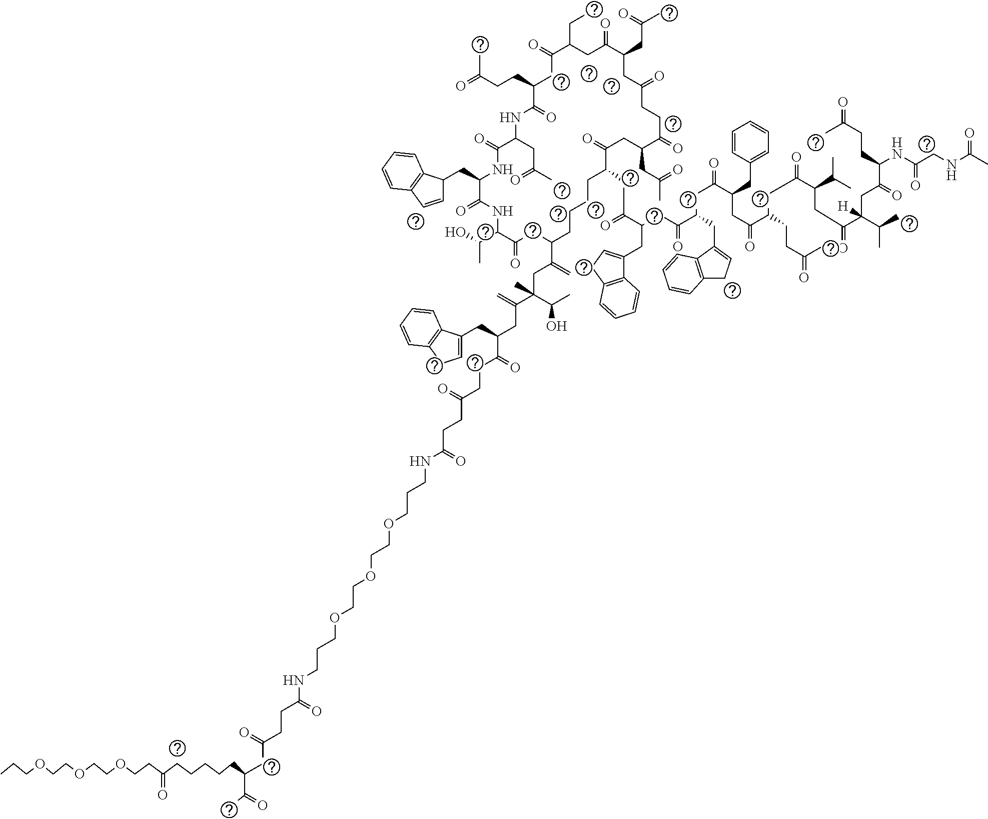 |
| PspSP1489 | [Ac-AETVEFWWCNGNSENWTCTW-GG-Ttds-K(Ttds-Ttds-PEG3)]2-PEG4-DBCO |
| | 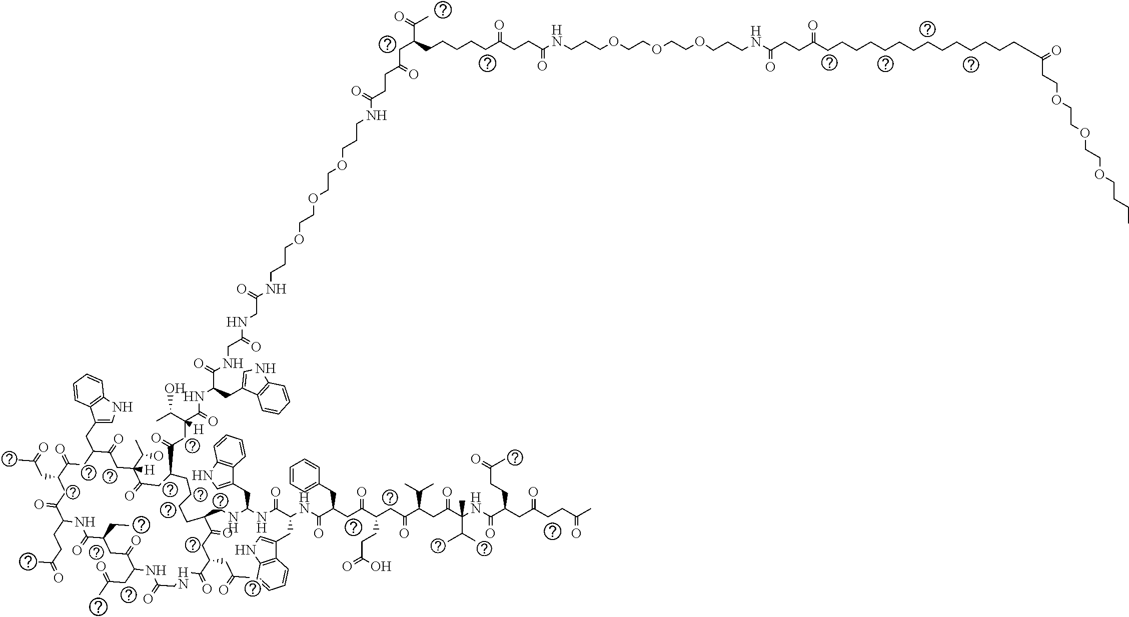 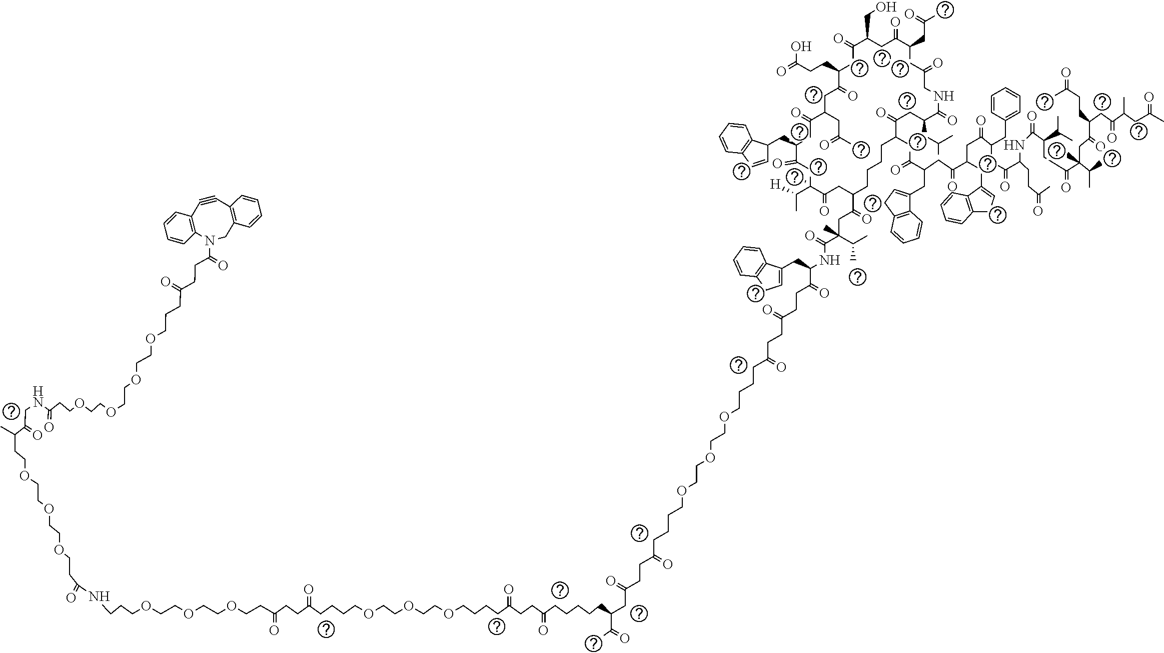 |
| PepSP1538 | Ac-AETVEFWWCNGNSENWTCTW-GG-Ttds-K(PEG4-DBCO-FEA)-NH2 |
| | 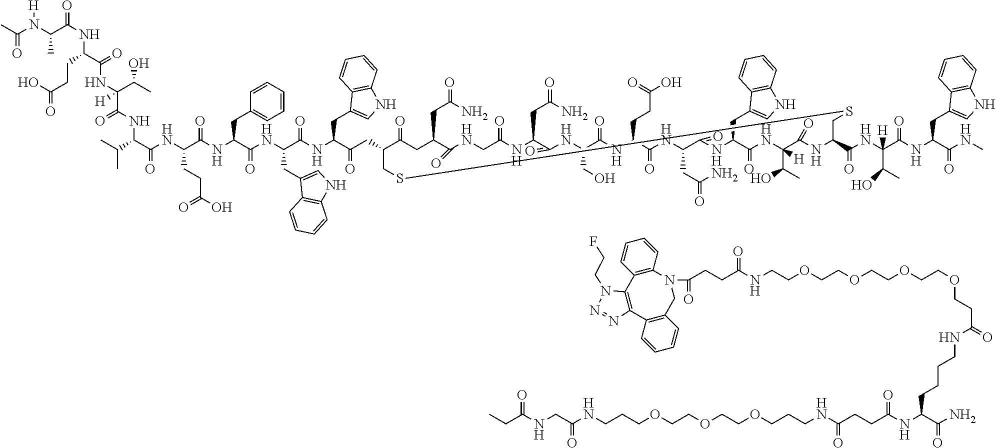 |
| PepSP1581 | [Ac-AETVEFWWCNGNSENWTCTW-GG-Ttds-K(PEG3)]2-PEG4-DBCO-FEA |
| | 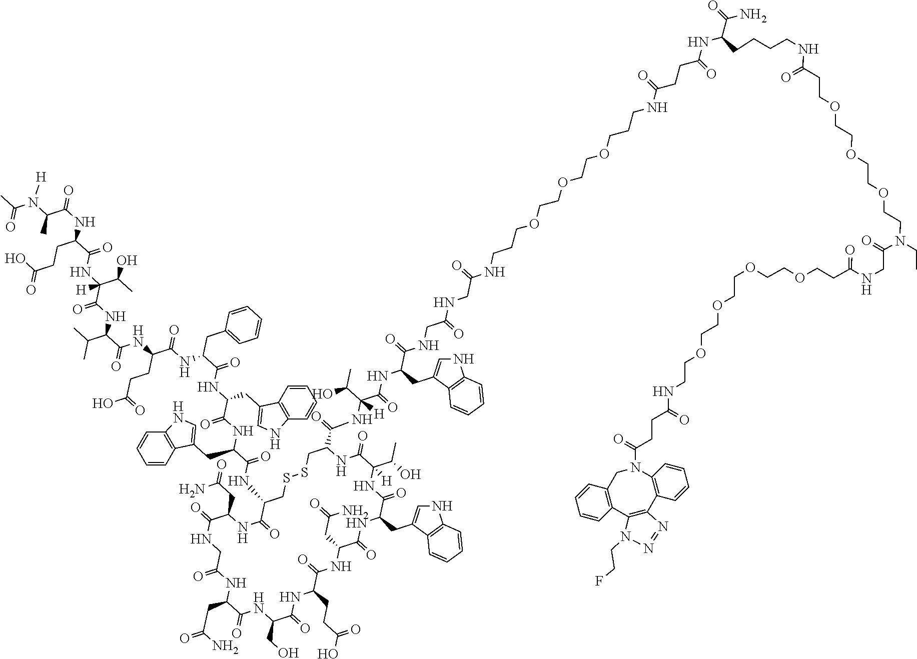 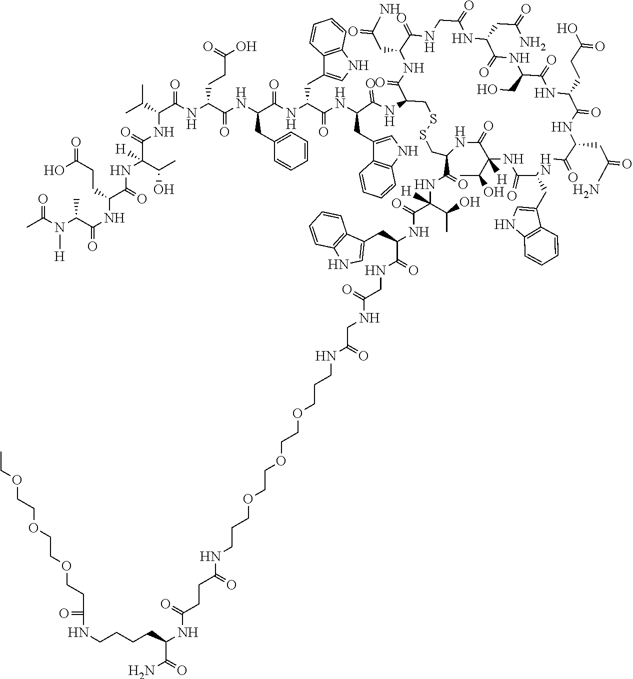 |
| PepSP1582 | [Ac-AETVEFWWCNGNSENWTCTW-GG-Ttds-K(PEG4-PEG3)]2-PEG4-DBCO-FEA |
| | 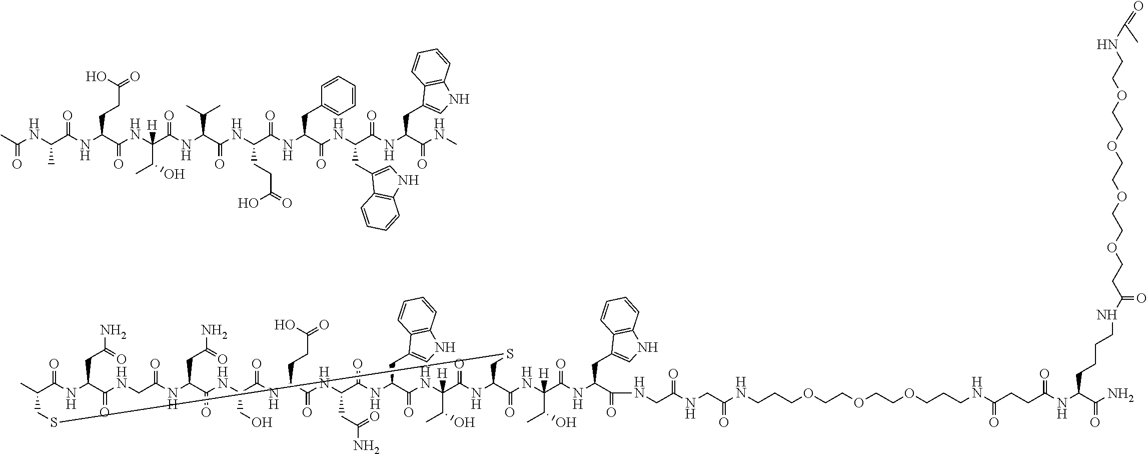 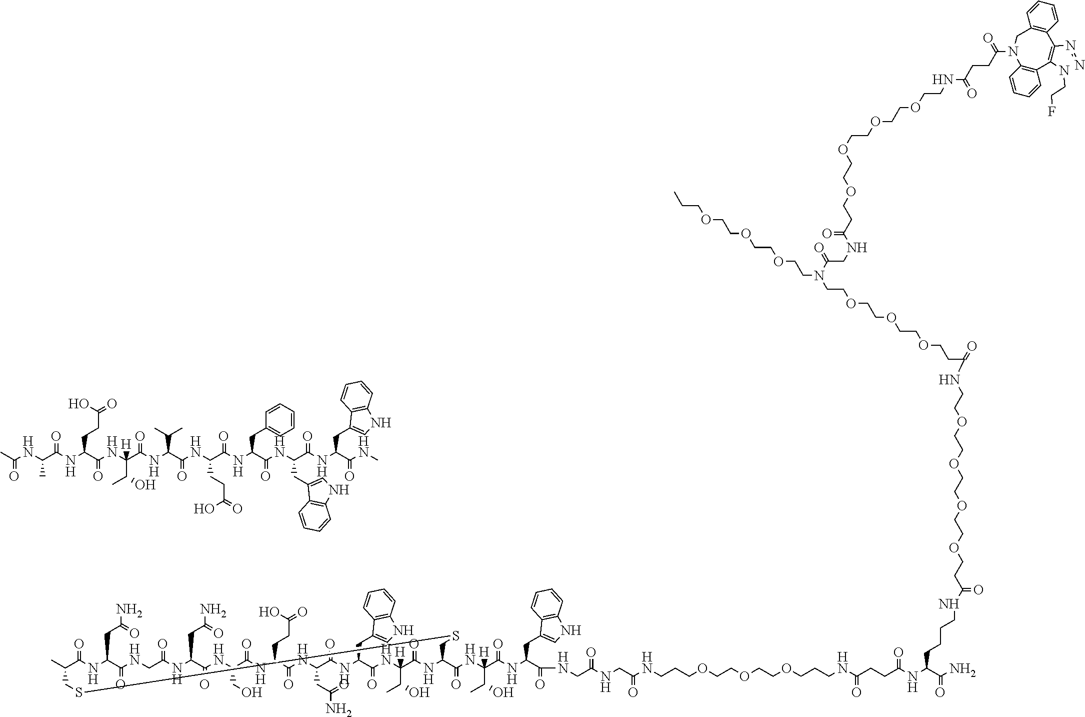 |
| PepSP1583 | [Ac-AETVEFWWCNGNSENWTCTW-GG-Ttds-K(Ttds-Ttds-PEG3)]2-PEG4-DBCO- |
| | 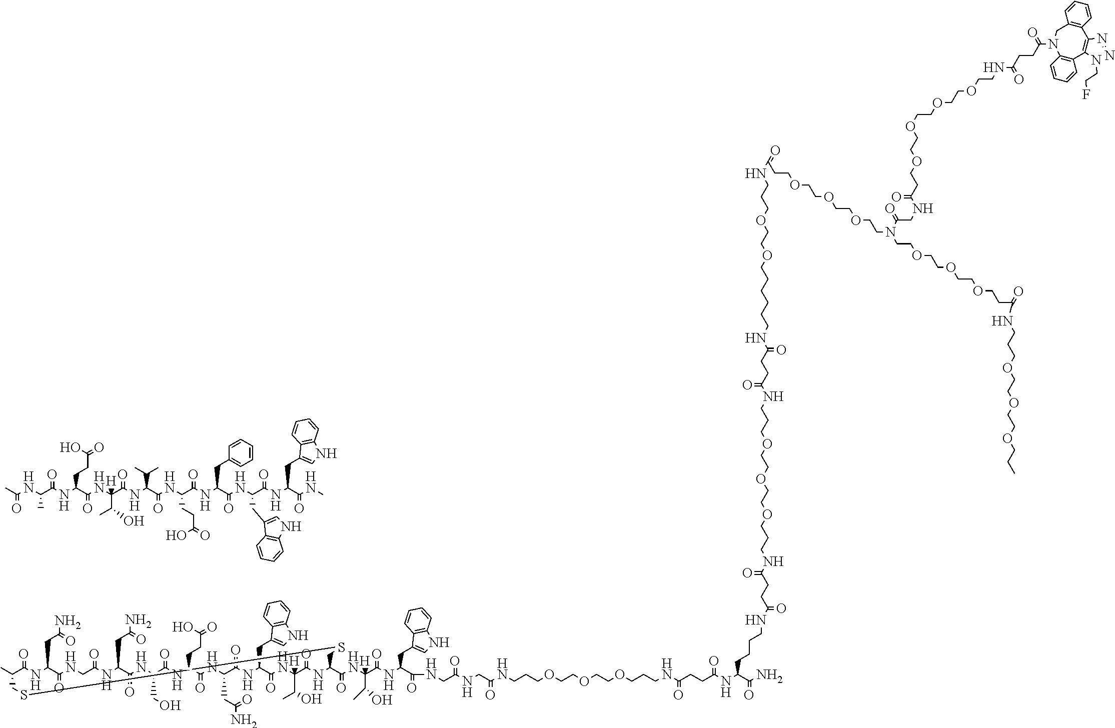 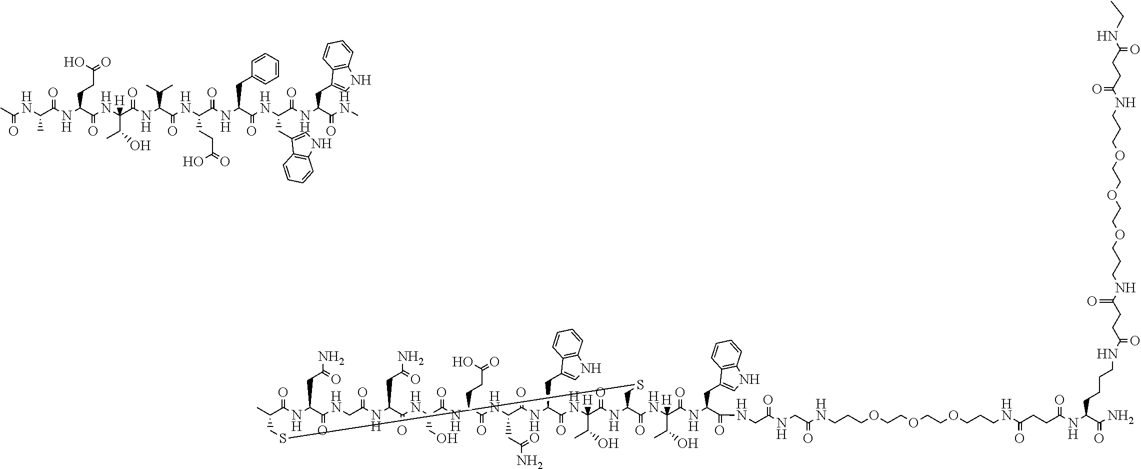 |

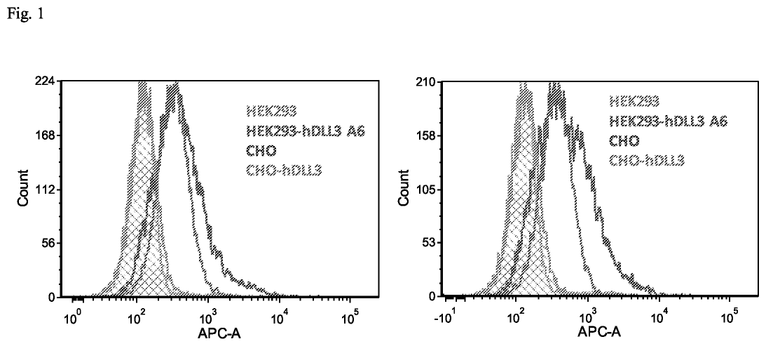

Peptides that that contain various linkers and/or are labeled with a fluorescent agent were tested for binding to DLL3 by BLI, SPR or flow cytometry using cells expressing human DLL3. The binding properties of the peptides are summarized in Table 6 below. As shown in the table below, the addition of linkers and/or fluorescent agent does not negatively impact the binding to DLL3. Fluorescent labeled peptides were also found to specifically bind to DLL3 protein expressed on the surface of cells (CHO cells); see FIG. 1.

TABLE 6
Binding characteristics

| PepSP ID | Kd | RT |
|---|---|---|
| 1215 | 11.6 | 681 |
| 1274 | 21.4 | 254 |
| 1321 | 36.8 | 369 |
| 1324 | 29 ± 16 | 189.3 ± 73 |
| 1342 | 42 ± 8 | 198.9 ± 41.1 |

The peptide dimers were tested for binding to DLL3. As shown in Table 7 below, dimerization greatly increased complex stability, resulting in a 30- to 100-fold more stable off rate. Dimers made using the bis-PEG18 linker showed the greatest improvement compared to the corresponding monomer. The dimeric peptides PepSP1270, 1271 and 1272 were also able to interact with native huDLL3 on the surface of cells (e.g., CHO cells) better than the corresponding monomer PepSP1146, as shown by their higher efficiency in competing the binding of the AF647-labelled PepSP1146. The peptide dimerized through the bis-PEG18 linker (PepSP1172) was shown to be the most efficient binder. The data in Table 7 below are kinetic parameters measured from curve fitting of the SPR sensorgrams of the peptides.

TABLE 7
Binding properties of the peptide dimers

| Pep-ID | linker | KD (nM) | kon (1/Ms) | koff (1/s) | IC50 |
|---|---|---|---|---|---|
| PepSP1270 | bis-PEG6 | 3 | 7.43E+04 | 2.4E−04 | 15.3 |
| PepSP1271 | bis-PEG14 | 3 | 1.04E+05 | 2.8E−04 | 10 |
| PepSP1272 | bis-PEG18 | 0.61 | 9.0E+04 | 5.5E−05 | 7.2 |
| PepSP1146 | n/a | 8.7E+04 | 5.4E−03 | 8.7E+04 | 76 |

Binding data of dimeric peptides PepSP1344, 1371, 1384, 1462, 1487, 1488 and 1489 are shown in Table 8 below. The data in the table were measured from curve fitting of the SPR sensorgrams of the peptides. As can be seen from the data, dimerization significantly improved binding to DLL3.

TABLE 8
Binding property of selected dimeric peptides
and the corresponding monomer peptides

| Pep ID | KD(nM) | kon(1/Ms) | koff (1/s) |
|---|---|---|---|
| PepSP1146 | 68.9 | 8.7E+04 | 5.4E−03 |
| PepSP1344 | 6 | 1.8E+05 | 1.0E−03 |
| PepSP1371 | 2.4 | 3.0E+05 | 5.7E−04 |
| PepSP1171 | 70 | 1.4E+05 | 9.7E−03 |
| PepSP1384 | 4.0 | 1.8E+05 | 6.6E−04 |
| PepSP1462 | 6.3 | 2.3E+05 | 1.4E−03 |
| PepSP1487 | 5.5 | 7.6E+04 | 4.2E−04 |
| PepSP1488 | 2.6 | 1.2E+05 | 3.1E−04 |
| PepSP1489 | 3.3 | 1.1E+05 | 3.7E−04 |

The four fluoro-labeled conjugates (PepSP1538, PepSP1581, PepSP1582 and PepSP1583) were tested at single point by SPR binding assay to rhDLL3-Fc and the complex stability (koff) of each molecule was measured. The results show the peptides as tight rhDLL3-Fc binders with a stable complex half-life (Table 9).

TABLE 9
Binding properties of labeled peptides

| Pep ID | koff ± SE (1/s) |
|---|---|
| PepSP1538 | 3.9 ± 0.05E−03 |
| PepSP1581 | 3.4 ± 0.02E−05 |
| PepSP1582 | 3.6 ± 0.09E−05 |
| PepSP1583 | 3.2 ± 0.08E−05 |

### Example 4 Binding and Cross-Reactivity of DLL3 Binding Peptides

Materials and Methods: DLL3 expressing cells used in the experiments included CHO cells expressing human (hu), cynomolgus monkey (cyno), mouse (mu), and rat DLL3. CHO DHFR-cells and CHO cells expressing human FLT3 were used as negative controls. DLL3 target expression was analyzed by flow cytometry. DLL3-expressing CHO cells or DLL3-negative CHO cells, as listed above, were suspended in FACS buffer (1×PBS+1% fetal bovine serum) and incubated with 10 μg/ml AMG 757 for 40 min at 4° C. Cells were then washed twice in FACS buffer and incubated with an anti-human IgG Fcγ antibody labeled with allophycocyanin for 20 min at 4° C. DLL3 cell surface expression was analyzed using an LSR Fortessa flow cytometer (Beckton Dickinson) and FACSDiva software (Becton Dickinson). The percent of CHO cells expressing DLL3 from different species is summarized in the table below.

| | | | | High | | | |
|---|---|---|---|---|---|---|---|
| | CHO | CHO | CHO | CHO | Low CHO | CHO | CHO |
| Cell Line | huDLL3 | cynoDLL3 | muDLL3 | rtDLL3 | rtDLL3 | DHFR− | huFTL3 |
| DLL3 | 99.7 | 98.4 | 99.5 | 98.6 | 98.6 | 12.3 | 3.3 |
| positive cells | | | | | | | |
| (%) | | | | | | | |

Peptide PepSP1462, identified herein was biotin labeled. The biotin labeled peptide was detected using an anti-streptavidin antibody conjugated to AF488. The AF488-labeled peptide was detected directly.

Binding of the labeled PepSP1462 peptide to DLL3-expressing cells was tested by flow cytometry. Binding specificity of the labeled peptide for DLL3 was assessed using DLL3-negative cell lines. DLL3-expressing and DLL3-negative cells were incubated in the absence or presence of 100 M PepSP1462-biotin peptide for 30 minutes at 4° C. Cells were then washed twice with FACS buffer and incubated with an anti-streptavidin antibody conjugated to AF488 (Thermo Fisher). As a control, cells were incubated with the anti-streptavidin-Alexa Fluor 488 antibody only. Cells were then washed twice with FACS buffer an resuspended in FACS buffer that contained 0.5 μg/ml propidium iodide. Cells were analyzed by flow cytometry, using an LSR Fortessa and FACSDiva software (Becton Dickinson) or Flow Jo (Flow Jo LLC).

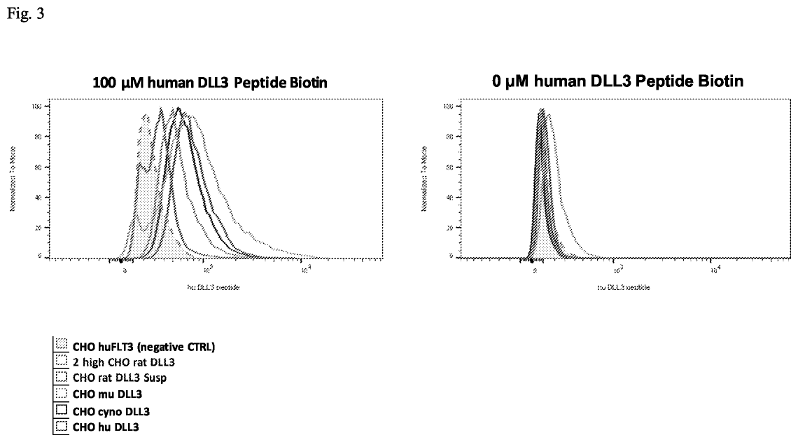

FIG. 3 shows that PepSP1462 labeled with biotin and detected with the anti-streptavidin-AF488 antibody binds to CHO cells that express human, mouse, cynomolgus monkey or rat DLL3, but does not bind to CHO cells that are not transfected with DLL3 (CHO huFLT3). Binding of the labeled peptide to DLL3 expressed on cells is shown as a histogram shift to the right of the negative control cell line. These data also indicate that PepSP1462 cross-reacts to DLL3 from other species.

The specification is most thoroughly understood in light of the teachings of the references cited within the specification. The embodiments within the specification provide an illustration of embodiments of the invention and should not be construed to limit the scope of the invention. The skilled artisan readily recognizes that many other embodiments are encompassed by the invention. All publications, patents, and sequences cited in this disclosure are incorporated by reference in their entirety. To the extent the material incorporated by reference contradicts or is inconsistent with this specification, the specification will supersede any such material. The citation of any references herein is not an admission that such references are prior art to the present invention.

Those skilled in the art will recognize, or be able to ascertain using no more than routine experimentation, many equivalents to the specific embodiments of the invention described herein. Such equivalents are intended to be encompassed by the following embodiments.

What is claimed:

1. A polypeptide comprising an amino acid sequence selected from
a) C-X1-X2-X3-X4-X5-X6-X7-X8-C(SEQ ID NO: 78), wherein
X1 is Y, H, T, K, S, W, D, E, L, N, Q, or R;
X2 is G, W, Y, M, T, or V;
X3 is D, N, Y, T, A, E, G, or S;
X4 is W, A, E, S, V, Y, D, G, N, P, Q, R, or T;
X5 is D, E, G, Y, N, W, K, R, or S;
X6 is E, D, G, N, T, A, Q, or V;
X7 is W, Y, V, E, or S; and
X8 is T, G, A, or S;
or
b) SEQ ID NO:6.

2. The polypeptide of claim 1, wherein
a) X1 is Y, H, T, W, or N;
X2 is G;
X3 is D, N, or T;
X4 is W, A, S, N, R, or T;
X5 is D, E, G, Y, N, or S;
X6 is E, D, or N;
X7 is W, Y, or E; and
X8 is T.

3. The polypeptide of claim 1, wherein the polypeptide comprises the amino acid sequence of any one of SEQ ID NOS: 1-23

4. The polypeptide of claim 1 or 2, wherein the polypeptide comprises the amino acid sequence of any one of SEQ ID NOS: 1-7.

5. The polypeptide of any one of claims 1-4, wherein the amino acid sequence further comprises the amino acid residues AETVEF or AETVE at the N-terminal of the amino acid sequence.

6. The polypeptide of claim 5, wherein the polypeptide comprises the amino acid sequence of any one of SEQ ID NOS: 39-61.

7. The polypeptide of claim 5 or 6, wherein the polypeptide comprises the amino acid sequence of any one of SEQ ID NOS: 39-45.

8. A polypeptide comprising the amino acid sequence of any one of SEQ ID NOS: 1-38.

9. The polypeptide of claim 8, wherein the amino acid sequence further comprises the amino acid residues AETVEF or AETVE at the N-terminal of the amino acid sequence.

10. The polypeptide of claim 9, wherein the polypeptide comprises the amino acid sequence of any one of SEQ ID NOS: 39-76, preferably SEQ ID NOS: 39-61, more preferably SEQ ID NOS: 39-45.

11. The polypeptide of any one of claims 1-10, wherein the polypeptide is modified at the N-terminus, the C-terminus or both.

12. The polypeptide of claim 11 wherein the amino acid residue at the N-terminus is acetylated.

13. The polypeptide of claim 11 or 12, wherein the C-terminus of the polypeptide is amidated or aminated.

14. The polypeptide of any one of claims 1-13, wherein the polypeptide comprises a dimer of the amino acid sequence.

15. The polypeptide of claim 14, wherein the dimer is a homodimer.

16. The polypeptide of claim 14 or 15, wherein the dimer comprises a first linker linking the two amino acid sequences.

17. The polypeptide of any one of claims 1-16, wherein the polypeptide further comprises a detectable agent.

18. The polypeptide of claim 17, wherein the detectable agent is linked to the polypeptide via a second linker, a chelating agent, or a combination thereof.

19. The polypeptide of any one of claims 16-18, wherein the first linker or the second linker is independently a peptide linker or a non-peptide linker.

20. The polypeptide of claim 19, wherein the first or second linker comprises non-natural amino acids.

21. The polypeptide of claim 20, wherein the first or the second linker independently comprises a poly(ethylene glycol) (PEG) linker.

22. The polypeptide of claim 21, wherein the PEG linker comprises PEG2, PEG3, PEG4, PEG6, Bis-PEG18, Bis-PEG16, Bis-PEG14, bis-PEG12, Bis-propargyl-PEG2, bis-propargyl-PEG6, bis-propargyl-PEG14, bis-propargyl-PEG18, or a combination thereof.

23. The polypeptide of any one of claims 16-22, wherein the first linker further comprises hPra, Lys(N)3, Trioxatridecan-succinamic acid (Ttds), Gly-Gly or a combination thereof.

24. The polypeptide of any one of claims 18, 19 or 21, wherein the second linker comprises a bicyclo[6.1.0]nonyne (BCN) group or a dibenzocyclooctyne (DBCO) group.

25. The polypeptide of claim 18, wherein the chelating agent is DOTA, TETA, DFO, NOTA, DTPA, HOPO, or Macropa.

26. The polypeptide of claim 17, wherein the detectable agent comprises a fluorescent agent or a radioisotope.

27. The polypeptide of claim 26, wherein the fluorescent agent is Cy3, Cy5, Fluoresceinisothiocyanate (FITC), Anthranilyl, 2-Aminobenzoyl (Abz), 5-Carboxyfluorescein (5-FAM), 6-Carboxyfluorescein (6-FAM), Carboxytetramethyl rhodamine (TAMRA), 5-(Dimethylamino) naphthalene-1-sulfonyl (Dansyl), 5-[(2-Aminoethyl)amino]naphthalene-1-sulfonic acid (EDANS), or 7-Methoxycoumarinyl-4-acetyl (Mca).

28. The polypeptide of claim 26, wherein the radioisotope is 67Ga, 99mTc, 111In, 68Ga, 64Cu, 44Sc, 86Y, 89Zr, 18F, 125I, 123I, 124I, or 203Pb.

29. The polypeptide of claim 28, wherein the radioisotope is 18F.

30. The polypeptide of claim 26, wherein the radioisotope is 47Sc, 114mIn, 177Lu, 90Y, 212/213Bi, 212Pb, 225Ac, 186/188Re, 67Cu, 131I, 227Th, 211At, or 90Y.

31. The polypeptide of any one of claims 1-30, wherein the polypeptide binds to DLL3.

32. The polypeptide of claim 31, wherein the polypeptide binds to human DLL3 expressed on the surface of a cell.

33. A pharmaceutical composition comprising the polypeptide of any one of claims 1-32.

34. The pharmaceutical composition of claim 33, where the composition further comprises N-tert-Butyl-α-phenylnitrone (PBN), ethanol, Sodium Ascorbate, gentisic acid, or a combination thereof.

35. The pharmaceutical composition of claim 33 or 34, wherein the composition has a pH of from 4.5 to 8.0.

36. A method of detecting DLL3 in a sample comprising contacting the polypeptide of any one of claims 17-32 or the pharmaceutical composition of any one of claims 33-35 with the sample, and detecting DLL3 in the sample.

37. The method of claim 36, wherein the sample comprises a cell expressing DLL3.

38. The method of claim 36 or 37, wherein the DLL3 is human DLL3.

39. The method of claim 38, wherein the cell is inside the body of a subject, and the method comprises administering the polypeptide or the pharmaceutical composition to the subject and detecting DLL3 in the subject using an imaging technique.

40. The method of claim 39, wherein the subject is a human with a DLL3-expressing tumor or cancer.

41. The method of claim 39 or 40, wherein the tumor or cancer is small cell lung cancer (SCLC), non-small cell lung cancer (NSCLC), glioma, glioblastoma, melanoma, neuroendocrine prostate cancer, neuroendocrine pancreatic cancer, hepatoblastoma, large cell pulmonary neuroendocrine cancer, pancreatic neuroendocrine cancer, bladder neuroendocrine cancer, gastric neuroendocrine cancer, adrenal exocrine tumors, Merkel cell carcinoma, neuroblastoma, head and neck carcinoid or neuroendocrine cancer, head and neck paraganglioma, or cervical small cell neuroendocrine cancer.

42. The method of any one of claims 39-41, wherein the imaging technique is positron emission tomography (PET) scan.

43. A method of treating a DLL3-expressing tumor or cancer disease, the method comprises administering to a subject in need thereof the polypeptide of claim 30 or the pharmaceutical composition of any one of claims 33-35.

44. The method of claim 43, wherein the subject is a human.

45. The method of any one of claims 38-44, wherein the administration is intravenous administration.
